SEQUENCE LISTING (25 sequences)

SEQ ID NO: 1 - protein, 119 aa, Homo sapiens
QVRLQESGPGLVKPSQTLSLTCTVSGGSISSGGYYWSWIRQHPGKGLEWIGYIYYSGSTYYNPSLESRVTISVDTSKNQFSLKLSSVTAADTAVYYCARYSSIDAFEIWGQGTMVTVSS

SEQ ID NO: 2 - protein, 130 aa, Homo sapiens
QVQLQQSGPGLVKPPQTLSLTCAISGDSVSSNSAAWNWIRQSPSRGLEWLGRTYYRSKWYNDYAVSVKSRITINPDTSKNQFSLQLNSVTPEDTAVYYCARDPYYYDSSGYYYFDAFDIWGQGTMVTVSS

SEQ ID NO: 3 - protein, 108 aa, Homo sapiens
SYVLTQPPSVSVAPGKTATITCGGDDIGSKSVHWYQQKPGQAPVLVVYDDGDRPSGIPERFSGSNSGNTATLAISRVEAGDEADYYCQVWDSSSDQYVFGSGTKLTVL

SEQ ID NO: 4 - protein, 108 aa, Homo sapiens
SYELTQPHSVSVAPGQTARITCGGDNIGSKSVHWYQQRPGQAPVLVVYDDSDRPSGIPERFSGSNSENTATLTISGVEAGDEADYYCQVWDSTSHHVVFGGGTKLTVL

SEQ ID NO: 5 - protein, 7 aa, Homo sapiens
SGGYYWS

SEQ ID NO: 6 - protein, 16 aa, Homo sapiens
YIYYSGSTYYNPSLES

SEQ ID NO: 7 - protein, 9 aa, Homo sapiens
YSSIDAFEI

SEQ ID NO: 8 - protein, 11 aa, Homo sapiens
GGDDIGSKSVH

SEQ ID NO: 9 - protein, 7 aa, Homo sapiens
DDGDRPS

SEQ ID NO: 10 - protein, 11 aa, Homo sapiens
QVWDSSSDQYV

SEQ ID NO: 11 - protein, 7 aa, Homo sapiens
SNSAAWN

SEQ ID NO: 12 - protein, 18 aa, Homo sapiens
RTYYRSKWYNDYAVSVKS

SEQ ID NO: 13 - protein, 18 aa, Homo sapiens
DPYYYDSSGYYYFDAFDI

SEQ ID NO: 14 - protein, 11 aa, Homo sapiens
GGDNIGSKSVH

SEQ ID NO: 15 - protein, 7 aa, Homo sapiens
DDSDRPS

SEQ ID NO: 16 - protein, 11 aa, Homo sapiens
QVWDSTSHHVV

SEQ ID NO: 17 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - protein, 248 aa, Homo sapiens
SYVLTQPPSVSVAPGKTATITCGGDDIGSKSVHWYQQKPGQAPVLVVYDDGDRPSGIPERFSGSNSGNTATLAISRVEAGDEADYYCQVWDSSSDQYVFGSGTKLTVLSGGSTITSYNVYYTKLSSSGTQVRLQESGPGLVKPSQTLSLTCTVSGGSISSGGYYWSWIRQHPGKGLEWIGYIYYSGSTYYNPSLESRVTISVDTSKNQFSLKLSSVTAADTAVYYCARYSSIDAFEIWGQGTMVTVSS

SEQ ID NO: 20 - protein, 259 aa, Homo sapiens
SYELTQPHSVSVAPGQTARITCGGDNIGSKSVHWYQQRPGQAPVLVVYDDSDRPSGIPERFSGSNSENTATLTISGVEAGDEADYYCQVWDSTSHHVVFGGGTKLTVLSGGSTITSYNVYYTKLSSSGTQVQLQQSGPGLVKPPQTLSLTCAISGDSVSSNSAAWNWIRQSPSRGLEWLGRTYYRSKWYNDYAVSVKSRITINPDTSKNQFSLQLNSVTPEDTAVYYCARDPYYYDSSGYYYFDAFDIWGQGTMVTVSS

SEQ ID NO: 21 - protein, 468 aa, Homo sapiens
MGWSCIILFLVATATGVHSQVRLQESGPGLVKPSQTLSLTCTVSGGSISSGGYYWSWIRQHPGKGLEWIGYIYYSGSTYYNPSLESRVTISVDTSKNQFSLKLSSVTAADTAVYYCARYSSIDAFEIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 479 aa, Homo sapiens
MGWSCIILFLVATATGVHSQVQLQQSGPGLVKPPQTLSLTCAISGDSVSSNSAAWNWIRQSPSRGLEWLGRTYYRSKWYNDYAVSVKSRITINPDTSKNQFSLQLNSVTPEDTAVYYCARDPYYYDSSGYYYFDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 233 aa, Homo sapiens
MGWSCIILFLVATATGVHSSYVLTQPPSVSVAPGKTATITCGGDDIGSKSVHWYQQKPGQAPVLVVYDDGDRPSGIPERFSGSNSGNTATLAISRVEAGDEADYYCQVWDSSSDQYVFGSGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 24 - protein, 233 aa, Homo sapiens
MGWSCIILFLVATATGVHSSYELTQPHSVSVAPGQTARITCGGDNIGSKSVHWYQQRPGQAPVLVVYDDSDRPSGIPERFSGSNSENTATLTISGVEAGDEADYYCQVWDSTSHHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

(19) United States
(12) Patent Application Publication
(10) Pub. No.: US 2025/0255980 A1
(43) Pub. Date: Aug. 14, 2025

(54) Antibody-Drug Conjugates Against the Glucose-regulating Protein 78 (GRP78)

(71) Applicant: MBrace Therapeutics, Inc., San Diego, CA (US)

(72) Inventor: Fernanda I. Staquicini, Jersey City, NJ (US)

(21) Appl. No.: 19/050,987

(22) Filed: Feb. 11, 2025

Related U.S. Application Data
(60) Provisional application No. 63/552,519, filed on Feb. 12, 2024.
(60) Provisional application No. 63/659,954, filed on Jun. 14, 2024.

(51) Int. Cl. A61K 47/68; A61P 35/00; C07K 16/28

(52) U.S. Cl. CPC A61K 47/68037; A61K 47/6889; A61P 35/00; C07K 16/28; C07K 2317/565

(57) ABSTRACT

This disclosure relates to antibodies, binding polypeptides, and immunoconjugates including antibody-drug conjugates (ADCs) having binding activity for human glucose-regulating protein 78 (GRP78), compositions comprising and methods for using as well as making the same.

### CROSS-REFERENCE TO RELATED APPLICATIONS

This application claims priority to U.S. Application No. 63/552,519 filed on Feb. 12, 2024; and U.S. Application No. 63/659,954 filed on Jun. 14, 2024; each of which are hereby incorporated into this application in their entireties.

### FIELD OF THE DISCLOSURE

This disclosure relates to antibodies, binding polypeptides, and immunoconjugates including antibody-drug conjugates (ADCs) having binding activity for human glucose-regulating protein 78 (GRP78), compositions comprising and methods for using as well as making the same.

### BACKGROUND

Glucose-Regulated Protein 78 (GRP78) is a chaperone heat shock protein that is a key protein of the unfolded protein response in the Endoplasmic Reticulum (ER) in normal cells. GRP78 promotes folding and assembly of proteins and regulates ER stress signaling through Ca²⁺ binding to the ER. In tumors, complex conditions in the tumor microenvironment lead to the upregulation of GRP78 and cell surface expression. In addition to tumors, other indications are also associated with the upregulation of GRP78 and cell surface expression.

Previous efforts to target GRP78 have been ineffective. Small molecule GRP78 modulators are not targeted to cancerous cells and therefor are generally unacceptably toxic to healthy cells and tissues. On the other hand, mAb-targeted GRP78 modulators have been exceedingly difficult to produce in view of toxicities to the expressing cells. Effective therapies against cancer and other indications characterized by the expression or overexpression of GRP78 therefore remain urgently needed for numerous cancer indication, including in particular castration resistant prostate cancer.

### SUMMARY OF THE INVENTION

Cancer therapies targeting tumor-specific targets like GRP78, like those disclosed herein, address the needs identified above. Additionally, therapies targeting GRP78 in non-cancer indications are also included within the scope of the invention.

This disclosure relates in certain aspects to antibodies, binding polypeptides, and immunoconjugates specific for human Glucose-regulating protein 78 (GRP78).

In certain aspects, this disclosure described antibody-drug conjugates, or ADCs (discussed in more detail below), comprising one or more of the antibodies or antibody fragments described herein.

In certain aspects, this disclosure provides an antibody or antigen-binding fragment thereof that specifically binds to an epitope of GRP78.

In certain aspects of this disclosure, the antibody or antigen-binding fragment comprises a heavy chain comprising a heavy chain variable region comprising an amino acid sequence having at least 85% sequence identity to SEQ ID NO: 1.

In certain aspects of this disclosure, the antibody or antigen-binding fragment comprises a light chain comprising a light chain variable region comprising an amino acid sequence having at least 85% sequence identity to SEQ ID NO: 2.

In certain aspects of this disclosure, the antibody or antigen-binding fragment comprises a heavy chain comprising a heavy chain variable region comprising an amino acid sequence having at least 85% sequence identity to SEQ ID NO: 3.

In certain aspects of this disclosure, the antibody or antigen-binding fragment comprises a light chain comprising a light chain variable region comprising an amino acid sequence having at least 85% sequence identity to SEQ ID NO: 4.

In certain aspects of this disclosure, the heavy chain variable region comprises three heavy chain complementarity-determining regions (HCDRs), wherein HCDR1 comprises the amino acid sequence set forth in SEQ ID NO: 5, HCDR2 comprises the amino acid sequence of SEQ ID NO: 6, and HCDR3 comprises the amino acid sequence set forth in SEQ ID NO: 7.

In certain aspects of this disclosure, the light chain variable region comprises three light chain complementarity-determining regions (LCDRs), wherein LCDR1 comprises the amino acid sequence set forth in SEQ ID NO: 8, LCDR2 comprises the amino acid sequence set forth in SEQ ID NO: 9, and LCDR3 comprises the amino acid sequence set forth in SEQ ID NO: 10.

In certain aspects of this disclosure, the heavy chain variable region comprises three heavy chain complementarity-determining regions (HCDRs), wherein HCDR1 comprises the amino acid sequence set forth in SEQ ID NO:11, HCDR2 comprises the amino acid sequence of SEQ ID NO: 12, and HCDR3 comprises the amino acid sequence set forth in SEQ ID NO: 13.

In certain aspects of this disclosure, the light chain variable region comprises three light chain complementarity-determining regions (LCDRs), wherein LCDR1 comprises the amino acid sequence set forth in SEQ ID NO: 14, LCDR2 comprises the amino acid sequence set forth in SEQ ID NO: 15, and LCDR3 comprises the amino acid sequence set forth in SEQ ID NO: 16.

In certain aspects, this disclosure provides the antibody or antigen-binding fragment of the above aspect or any other aspect or embodiment disclosed herein.

In certain aspects of this disclosure, the heavy chain variable region comprises an HCDR1 comprising the amino acid sequence set forth in SEQ ID NO: 5, an HCDR2 comprising the amino acid sequence set forth in SEQ ID NO: 6, and an HCDR3 comprising the amino acid sequence set forth in SEQ ID NO: 7; and the light chain variable region comprises an LCDR1 comprising the amino acid sequence set forth in SEQ ID NO: 8, an LCDR2 comprising the amino acid sequence set forth in SEQ ID NO: 9, and an LCDR3 comprising the amino acid sequence set forth in SEQ ID NO: 10.

In certain aspects of this disclosure, the heavy chain variable region comprises an HCDR1 comprising the amino acid sequence set forth in SEQ ID NO:11, an HCDR2 comprising the amino acid sequence set forth in SEQ ID NO: 12, and an HCDR3 comprising the amino acid sequence set forth in SEQ ID NO: 13; and the light chain variable region comprises an LCDR1 comprising the amino acid sequence set forth in SEQ ID NO: 14, an LCDR2 comprising the amino acid sequence set forth in SEQ ID NO: 15, and an LCDR3 comprising the amino acid sequence set forth in SEQ ID NO: 16.

In certain aspects of this disclosure, the amino acid sequence of the antibody has at least about 85%, 90%, 91%, 92%, 93%, 94%, 95%, 96%, 97%, 98%, or 99% sequence identity to the amino acid sequence SEQ ID NO: 19.

In certain aspects of this disclosure, the amino acid sequence of the antibody has at least about 85%, 90%, 91%, 92%, 93%, 94%, 95%, 96%, 97%, 98%, or 99% sequence identity to the amino acid sequence SEQ ID NO: 20.

In certain embodiments of the above aspects or any other aspect or embodiment disclosed herein, the light chain variable region comprises one or more of the amino acid sequence selected from the group consisting of SEQ ID NOs: 8-10 or 14-16.

In certain embodiments of the above aspects or any other aspect or embodiment disclosed herein, the heavy chain variable region is set forth in SEQ ID NO: 1 and the light chain variable region is set forth in SEQ ID NO: 3.

In certain embodiments of the above aspects or any other aspect or embodiment disclosed herein, the heavy chain variable region is set forth in SEQ ID NO: 2 and the light chain variable region is set forth in SEQ ID NO: 4.

In certain embodiments of the above aspects or any other aspect or embodiment disclosed herein, the heavy chain variable region is set forth in SEQ ID NO: 2 and the light chain variable region comprises the sequence set forth in SEQ ID NO: 14.

In certain embodiments of the above aspects or any other aspect or embodiment disclosed herein, the heavy chain variable region is set forth in SEQ ID NO: 2 and the light chain variable region comprises the sequence set forth in SEQ ID NO: 15.

In certain embodiments of the above aspects or any other aspect or embodiment disclosed herein, the heavy chain variable region is set forth in SEQ ID NO: 2 and the light chain variable region comprises the sequence set forth in SEQ ID NO: 16.

In certain embodiments of the above aspects or any other aspect or embodiment disclosed herein, the heavy chain variable region is set forth in SEQ ID NO: 1 and the light chain variable region comprises the sequence set forth in SEQ ID NO: 14.

In certain embodiments of the above aspects or any other aspect or embodiment disclosed herein, the heavy chain variable region is set forth in SEQ ID NO: 1 and the light chain variable region comprises the sequence set forth in SEQ ID NO: 15.

In certain embodiments of the above aspects or any other aspect or embodiment disclosed herein, the heavy chain variable region is set forth in SEQ ID NO: 1 and the light chain variable region comprises the sequence set forth in SEQ ID NO: 16.

In certain embodiments of the above aspects or any other aspect or embodiment disclosed herein, the heavy chain variable region is set forth in SEQ ID NO: 2 and the light chain variable region comprises the sequence set forth in SEQ ID NO: 8.

In certain embodiments of the above aspects or any other aspect or embodiment disclosed herein, the heavy chain variable region is set forth in SEQ ID NO: 2 and the light chain variable region comprises the sequence set forth in SEQ ID NO: 9.

In certain embodiments of the above aspects or any other aspect or embodiment disclosed herein, the heavy chain variable region is set forth in SEQ ID NO: 2 and the light chain variable region comprises the sequence set forth in SEQ ID NO: 10.

In certain embodiments of the above aspects or any other aspect or embodiment disclosed herein, the heavy chain variable region is set forth in SEQ ID NO: 1 and the light chain variable region comprises the sequence set forth in SEQ ID NO: 8.

In certain embodiments of the above aspects or any other aspect or embodiment disclosed herein, the heavy chain variable region is set forth in SEQ ID NO: 1 and the light chain variable region comprises the sequence set forth in SEQ ID NO: 9.

In certain embodiments of the above aspects or any other aspect or embodiment disclosed herein, the heavy chain variable region is set forth in SEQ ID NO: 1 and the light chain variable region comprises the sequence set forth in SEQ ID NO: 10.

In certain embodiments of the above aspects or any other aspect or embodiment disclosed herein, the heavy chain variable region comprises the sequence set forth in SEQ ID NO: 5 and the light chain variable region comprises the sequence set forth in SEQ ID NO: 8.

In certain embodiments of the above aspects or any other aspect or embodiment disclosed herein, the heavy chain variable region comprises the sequence set forth in SEQ ID NO: 5 and the light chain variable region comprises the sequence set forth in SEQ ID NO: 9.

In certain embodiments of the above aspects or any other aspect or embodiment disclosed herein, the heavy chain variable region comprises the sequence set forth in SEQ ID NO: 5 and the light chain variable region comprises the sequence set forth in SEQ ID NO: 10.

In certain embodiments of the above aspects or any other aspect or embodiment disclosed herein, the heavy chain variable region comprises the sequence set forth in SEQ ID NO: 5 and the light chain variable region comprises the sequence set forth in SEQ ID NO: 14.

In certain embodiments of the above aspects or any other aspect or embodiment disclosed herein, the heavy chain variable region comprises the sequence set forth in SEQ ID NO: 5 and the light chain variable region comprises the sequence set forth in SEQ ID NO: 15.

In certain embodiments of the above aspects or any other aspect or embodiment disclosed herein, the heavy chain variable region comprises the sequence set forth in SEQ ID NO: 5 and the light chain variable region comprises the sequence set forth in SEQ ID NO: 16.

In certain embodiments of the above aspects or any other aspect or embodiment disclosed herein, the heavy chain variable region comprises the sequence set forth in SEQ ID NO: 6 and the light chain variable region comprises the sequence set forth in SEQ ID NO: 8.

In certain embodiments of the above aspects or any other aspect or embodiment disclosed herein, the heavy chain variable region comprises the sequence set forth in SEQ ID NO: 6 and the light chain variable region comprises the sequence set forth in SEQ ID NO: 9.

In certain embodiments of the above aspects or any other aspect or embodiment disclosed herein, the heavy chain variable region comprises the sequence set forth in SEQ ID NO: 6 and the light chain variable region comprises the sequence set forth in SEQ ID NO: 10.

In certain embodiments of the above aspects or any other aspect or embodiment disclosed herein, the heavy chain variable region comprises the sequence set forth in SEQ ID NO: 6 and the light chain variable region comprises the sequence set forth in SEQ ID NO: 14.

In certain embodiments of the above aspects or any other aspect or embodiment disclosed herein, the heavy chain variable region comprises the sequence set forth in SEQ ID NO: 6 and the light chain variable region comprises the sequence set forth in SEQ ID NO: 15.

In certain embodiments of the above aspects or any other aspect or embodiment disclosed herein, the heavy chain variable region comprises the sequence set forth in SEQ ID NO: 6 and the light chain variable region comprises the sequence set forth in SEQ ID NO: 16.

In certain embodiments of the above aspects or any other aspect or embodiment disclosed herein, the heavy chain variable region comprises the sequence set forth in SEQ ID NO: 7 and the light chain variable region comprises the sequence set forth in SEQ ID NO: 8.

In certain embodiments of the above aspects or any other aspect or embodiment disclosed herein, the heavy chain variable region comprises the sequence set forth in SEQ ID NO: 7 and the light chain variable region comprises the sequence set forth in SEQ ID NO: 9.

In certain embodiments of the above aspects or any other aspect or embodiment disclosed herein, the heavy chain variable region comprises the sequence set forth in SEQ ID NO: 7 and the light chain variable region comprises the sequence set forth in SEQ ID NO: 10.

In certain embodiments of the above aspects or any other aspect or embodiment disclosed herein, the heavy chain variable region comprises the sequence set forth in SEQ ID NO: 7 and the light chain variable region comprises the sequence set forth in SEQ ID NO: 14.

In certain embodiments of the above aspects or any other aspect or embodiment disclosed herein, the heavy chain variable region comprises the sequence set forth in SEQ ID NO: 7 and the light chain variable region comprises the sequence set forth in SEQ ID NO: 15.

In certain embodiments of the above aspects or any other aspect or embodiment disclosed herein, the heavy chain variable region comprises the sequence set forth in SEQ ID NO: 7 and the light chain variable region comprises the sequence set forth in SEQ ID NO: 16.

In certain embodiments of the above aspects or any other aspect or embodiment disclosed herein, the heavy chain variable region comprises the sequence set forth in SEQ ID NO: 11 and the light chain variable region comprises the sequence set forth in SEQ ID NO: 8.

In certain embodiments of the above aspects or any other aspect or embodiment disclosed herein, the heavy chain variable region comprises the sequence set forth in SEQ ID NO: 11 and the light chain variable region comprises the sequence set forth in SEQ ID NO: 9.

In certain embodiments of the above aspects or any other aspect or embodiment disclosed herein, the heavy chain variable region comprises the sequence set forth in SEQ ID NO: 11 and the light chain variable region comprises the sequence set forth in SEQ ID NO: 10.

In certain embodiments of the above aspects or any other aspect or embodiment disclosed herein, the heavy chain variable region comprises the sequence set forth in SEQ ID NO: 11 and the light chain variable region comprises the sequence set forth in SEQ ID NO: 14.

In certain embodiments of the above aspects or any other aspect or embodiment disclosed herein, the heavy chain variable region comprises the sequence set forth in SEQ ID NO: 11 and the light chain variable region comprises the sequence set forth in SEQ ID NO: 15.

In certain embodiments of the above aspects or any other aspect or embodiment disclosed herein, the heavy chain variable region comprises the sequence set forth in SEQ ID NO: 11 and the light chain variable region comprises the sequence set forth in SEQ ID NO: 16.

In certain embodiments of the above aspects or any other aspect or embodiment disclosed herein, the heavy chain variable region comprises the sequence set forth in SEQ ID NO: 12 and the light chain variable region comprises the sequence set forth in SEQ ID NO: 8.

In certain embodiments of the above aspects or any other aspect or embodiment disclosed herein, the heavy chain variable region comprises the sequence set forth in SEQ ID NO: 12 and the light chain variable region comprises the sequence set forth in SEQ ID NO: 9.

In certain embodiments of the above aspects or any other aspect or embodiment disclosed herein, the heavy chain variable region comprises the sequence set forth in SEQ ID NO: 12 and the light chain variable region comprises the sequence set forth in SEQ ID NO: 10.

In certain embodiments of the above aspects or any other aspect or embodiment disclosed herein, the heavy chain variable region comprises the sequence set forth in SEQ ID NO: 12 and the light chain variable region comprises the sequence set forth in SEQ ID NO: 14.

In certain embodiments of the above aspects or any other aspect or embodiment disclosed herein, the heavy chain variable region comprises the sequence set forth in SEQ ID NO: 12 and the light chain variable region comprises the sequence set forth in SEQ ID NO: 15.

In certain embodiments of the above aspects or any other aspect or embodiment disclosed herein, the heavy chain variable region comprises the sequence set forth in SEQ ID NO: 12 and the light chain variable region comprises the sequence set forth in SEQ ID NO: 16.

In certain embodiments of the above aspects or any other aspect or embodiment disclosed herein, the heavy chain variable region comprises the sequence set forth in SEQ ID NO: 13 and the light chain variable region comprises the sequence set forth in SEQ ID NO: 8.

In certain embodiments of the above aspects or any other aspect or embodiment disclosed herein, the heavy chain variable region comprises the sequence set forth in SEQ ID NO: 13 and the light chain variable region comprises the sequence set forth in SEQ ID NO: 9.

In certain embodiments of the above aspects or any other aspect or embodiment disclosed herein, the heavy chain variable region comprises the sequence set forth in SEQ ID NO: 13 and the light chain variable region comprises the sequence set forth in SEQ ID NO: 10.

In certain embodiments of the above aspects or any other aspect or embodiment disclosed herein, the heavy chain variable region comprises the sequence set forth in SEQ ID NO: 13 and the light chain variable region comprises the sequence set forth in SEQ ID NO: 14.

In certain embodiments of the above aspects or any other aspect or embodiment disclosed herein, the heavy chain variable region comprises the sequence set forth in SEQ ID NO: 13 and the light chain variable region comprises the sequence set forth in SEQ ID NO: 15.

In certain embodiments of the above aspects or any other aspect or embodiment disclosed herein, the heavy chain variable region comprises the sequence set forth in SEQ ID NO: 13 and the light chain variable region comprises the sequence set forth in SEQ ID NO: 16.

In certain embodiments of the above aspects or any other aspect or embodiment disclosed herein, the heavy chain variable region comprises a sequence that has at least 90% sequence identity to the sequence set forth in SEQ ID NO: 1 and the light chain variable comprises a sequence that has at least 90% sequence identity to the sequence set forth in SEQ ID NO: 3.

In certain embodiments of the above aspects or any other aspect or embodiment disclosed herein, the heavy chain variable comprises a sequence that has at least 90% sequence identity to the sequence set forth in SEQ ID NO: 2 and the light chain variable comprises a sequence that has at least 90% sequence identity to the sequence set forth in SEQ ID NO: 4.

In certain embodiments of the above aspects or any other aspect or embodiment disclosed herein, the heavy chain variable comprises a sequence that has at least 90% sequence identity to the sequence set forth in SEQ ID NO: 2 and the light chain variable region comprises a sequence that has at least 90% sequence identity to the sequence set forth in SEQ ID NO: 14.

In certain embodiments of the above aspects or any other aspect or embodiment disclosed herein, the heavy chain variable comprises a sequence that has at least 90% sequence identity to the sequence set forth in SEQ ID NO: 2 and the light chain variable region comprises a sequence that has at least 90% sequence identity to the sequence set forth in SEQ ID NO: 15.

In certain embodiments of the above aspects or any other aspect or embodiment disclosed herein, the heavy chain variable comprises a sequence that has at least 90% sequence identity to the sequence set forth in SEQ ID NO: 2 and the light chain variable region comprises a sequence that has at least 90% sequence identity to the sequence set forth in SEQ ID NO: 16.

In certain embodiments of the above aspects or any other aspect or embodiment disclosed herein, the heavy chain variable comprises a sequence that has at least 90% sequence identity to the sequence set forth in SEQ ID NO: 1 and the light chain variable region comprises a sequence that has at least 90% sequence identity to the sequence set forth in SEQ ID NO: 14.

In certain embodiments of the above aspects or any other aspect or embodiment disclosed herein, the heavy chain variable comprises a sequence that has at least 90% sequence identity to the sequence set forth in SEQ ID NO: 1 and the light chain variable region comprises a sequence that has at least 90% sequence identity to the sequence set forth in SEQ ID NO: 15.

In certain embodiments of the above aspects or any other aspect or embodiment disclosed herein, the heavy chain variable comprises a sequence that has at least 90% sequence identity to the sequence set forth in SEQ ID NO: 1 and the light chain variable region comprises a sequence that has at least 90% sequence identity to the sequence set forth in SEQ ID NO: 16.

In certain embodiments of the above aspects or any other aspect or embodiment disclosed herein, the heavy chain variable comprises a sequence that has at least 90% sequence identity to the sequence set forth in SEQ ID NO: 2 and the light chain variable region comprises a sequence that has at least 90% sequence identity to the sequence set forth in SEQ ID NO: 8.

In certain embodiments of the above aspects or any other aspect or embodiment disclosed herein, the heavy chain variable comprises a sequence that has at least 90% sequence identity to the sequence set forth in SEQ ID NO: 2 and the light chain variable region comprises a sequence that has at least 90% sequence identity to the sequence set forth in SEQ ID NO: 9.

In certain embodiments of the above aspects or any other aspect or embodiment disclosed herein, the heavy chain variable comprises a sequence that has at least 90% sequence identity to the sequence set forth in SEQ ID NO: 2 and the light chain variable region comprises a sequence that has at least 90% sequence identity to the sequence set forth in SEQ ID NO: 10.

In certain embodiments of the above aspects or any other aspect or embodiment disclosed herein, the heavy chain variable comprises a sequence that has at least 90% sequence identity to the sequence set forth in SEQ ID NO: 1 and the light chain variable region comprises a sequence that has at least 90% sequence identity to the sequence set forth in SEQ ID NO: 8.

In certain embodiments of the above aspects or any other aspect or embodiment disclosed herein, the heavy chain variable comprises a sequence that has at least 90% sequence identity to the sequence set forth in SEQ ID NO: 1 and the light chain variable region comprises a sequence that has at least 90% sequence identity to the sequence set forth in SEQ ID NO: 9.

In certain embodiments of the above aspects or any other aspect or embodiment disclosed herein, the heavy chain variable comprises a sequence that has at least 90% sequence identity to the sequence set forth in SEQ ID NO: 1 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 8.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 14.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 15.

In certain embodiments of the above aspects or any other aspect or embodiment disclosed herein, the heavy chain variable region comprises a sequence that has at least 90% sequence identity to the sequence set forth in SEQ ID NO: 5 and the light chain variable region comprises a sequence that has at least 90% sequence identity to the sequence set forth in SEQ ID NO: 16.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 8.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 14.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 15.

In certain embodiments of the above aspects or any other aspect or embodiment disclosed herein, the heavy chain variable region comprises a sequence that has at least 90% sequence identity to the sequence set forth in SEQ ID NO: 6 and the light chain variable region comprises a sequence that has at least 90% sequence identity to the sequence set forth in SEQ ID NO: 16.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 8.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 14.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 15.

In certain embodiments of the above aspects or any other aspect or embodiment disclosed herein, the heavy chain variable region comprises a sequence that has at least 90% sequence identity to the sequence set forth in SEQ ID NO: 7 and the light chain variable region comprises a sequence that has at least 90% sequence identity to the sequence set forth in SEQ ID NO: 16.

In certain embodiments of the above aspects or any other aspect or embodiment disclosed herein, the heavy chain variable region comprises a sequence that has at least 90% sequence identity to the sequence set forth in SEQ ID NO: 11 and the light chain variable region comprises a sequence that has at least 90% sequence identity to the sequence set forth in SEQ ID NO: 8.

In certain embodiments of the above aspects or any other aspect or embodiment disclosed herein, the heavy chain variable region comprises a sequence that has at least 90% sequence identity to the sequence set forth in SEQ ID NO: 11 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 11 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 11 and the light chain variable region comprises a sequence that has at least 90% sequence identity to the sequence set forth in SEQ ID NO: 14.

In certain embodiments of the above aspects or any other aspect or embodiment disclosed herein, the heavy chain variable region comprises a sequence that has at least 90% sequence identity to the sequence set forth in SEQ ID NO: 11 and the light chain variable region comprises a sequence that has at least 90% sequence identity to the sequence set forth in SEQ ID NO: 15.

In certain embodiments of the above aspects or any other aspect or embodiment disclosed herein, the heavy chain variable region comprises a sequence that has at least 90% sequence identity to the sequence set forth in SEQ ID NO: 11 and the light chain variable region comprises a sequence that has at least 90% sequence identity to the sequence set forth in SEQ ID NO: 16.

In certain embodiments of the above aspects or any other aspect or embodiment disclosed herein, the heavy chain variable region comprises a sequence that has at least 90% sequence identity to the sequence set forth in SEQ ID NO: 12 and the light chain variable region comprises a sequence that has at least 90% sequence identity to the sequence set forth in SEQ ID NO: 8.

In certain embodiments of the above aspects or any other aspect or embodiment disclosed herein, the heavy chain variable region comprises a sequence that has at least 90% sequence identity to the sequence set forth in SEQ ID NO: 12 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 12 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 12 and the light chain variable region comprises a sequence that has at least 90% sequence identity to the sequence set forth in SEQ ID NO: 14.

In certain embodiments of the above aspects or any other aspect or embodiment disclosed herein, the heavy chain variable region comprises a sequence that has at least 90% sequence identity to the sequence set forth in SEQ ID NO: 12 and the light chain variable region comprises a sequence that has at least 90% sequence identity to the sequence set forth in SEQ ID NO: 15.

In certain embodiments of the above aspects or any other aspect or embodiment disclosed herein, the heavy chain variable region comprises a sequence that has at least 90% sequence identity to the sequence set forth in SEQ ID NO: 12 and the light chain variable region comprises a sequence that has at least 90% sequence identity to the sequence set forth in SEQ ID NO: 16.

In certain embodiments of the above aspects or any other aspect or embodiment disclosed herein, the heavy chain variable region comprises a sequence that has at least 90% sequence identity to the sequence set forth in SEQ ID NO: 13 and the light chain variable region comprises a sequence that has at least 90% sequence identity to the sequence set forth in SEQ ID NO: 8.

In certain embodiments of the above aspects or any other aspect or embodiment disclosed herein, the heavy chain variable region comprises a sequence that has at least 90% sequence identity to the sequence set forth in SEQ ID NO: 13 and the light chain variable region comprises a sequence that has at least 90% sequence identity to the sequence set forth in SEQ ID NO: 9.

In certain embodiments of the above aspects or any other aspect or embodiment disclosed herein, the heavy chain variable region comprises a sequence that has at least 90% sequence identity to the sequence set forth in SEQ ID NO: 13 and the light chain variable region comprises a sequence that has at least 90% sequence identity to the sequence set forth in SEQ ID NO: 10.

In certain embodiments of the above aspects or any other aspect or embodiment disclosed herein, the heavy chain variable region comprises a sequence that has at least 90% sequence identity to the sequence set forth in SEQ ID NO: 13 and the light chain variable region comprises a sequence that has at least 90% sequence identity to the sequence set forth in SEQ ID NO: 14.

In certain embodiments of the above aspects or any other aspect or embodiment disclosed herein, the heavy chain variable region comprises a sequence that has at least 90% sequence identity to the sequence set forth in SEQ ID NO: 13 and the light chain variable region comprises a sequence that has at least 90% sequence identity to the sequence set forth in SEQ ID NO: 15.

In certain embodiments of the above aspects or any other aspect or embodiment disclosed herein, the heavy chain variable region comprises a sequence that has at least 90% sequence identity to the sequence set forth in SEQ ID NO: 13 and the light chain variable region comprises a sequence that has at least 90% sequence identity to the sequence set forth in SEQ ID NO: 16.

In certain aspects, this disclosure provides an antibody or antigen-binding fragment thereof that specifically binds to an epitope of GRP78.

In certain embodiments of the above aspects or any other aspect or embodiment disclosed herein, the antibody or antigen-binding fragment comprises a heavy chain comprising a heavy chain variable region comprising an amino acid sequence having at least 85% sequence identity to SEQ ID NO: 1 or SEQ ID NO: 2.

In certain aspects of this disclosure, the antibody or antigen-binding fragment comprises a light chain comprising a light chain variable region comprising an amino acid sequence having at least 85% sequence identity to SEQ ID NO: 3 or SEQ ID NO: 4.

In certain aspects of this disclosure, the heavy chain variable region comprises three heavy chain complementarity-determining regions (HCDRs), wherein HCDR1 comprises the amino acid sequence set forth in SEQ ID NO: 11, HCDR2 comprises the amino acid sequence of SEQ ID NO: 12, and HCDR3 comprises the amino acid sequence set forth in SEQ ID NO: 13.

In certain aspects of this disclosure, the heavy chain variable region comprises three heavy chain complementarity-determining regions (HCDRs), wherein HCDR1 comprises the amino acid sequence set forth in SEQ ID NO: 5, HCDR2 comprises the amino acid sequence of SEQ ID NO: 6, and HCDR3 comprises the amino acid sequence set forth in SEQ ID NO: 7.

In certain aspects of this disclosure, the light chain variable region comprises three light chain complementarity-determining regions (LCDRs), wherein LCDR1 comprises the amino acid sequence selected from the group consisting of SEQ ID NO: 14, LCDR2 comprises the amino acid sequence set forth in SEQ ID NO: 15, and LCDR3 comprises the amino acid sequence set forth in SEQ ID NO: 16.

In certain aspects of this disclosure, the light chain variable region comprises three light chain complementarity-determining regions (LCDRs), wherein LCDR1 comprises the amino acid sequence selected from the group consisting of SEQ ID NO: 8, LCDR2 comprises the amino acid sequence set forth in SEQ ID NO: 9, and LCDR3 comprises the amino acid sequence set forth in SEQ ID NO: 10.

In certain aspects, this disclosure provides the antibody or antigen-binding fragment of the above aspect or any other aspect or embodiment disclosed herein.

In certain aspects of this disclosure, the heavy chain variable region comprises an HCDR1 comprising or the amino acid sequence set forth in SEQ ID NO:11, an HCDR2 comprising the amino acid sequence set forth in SEQ ID NO: 12, and an HCDR3 comprising the amino acid sequence set forth in SEQ ID NO: 13; and the light chain variable region comprises an LCDR1 comprising the amino acid sequence set forth in SEQ ID NO: 14, an LCDR2 comprising the amino acid sequence set forth in SEQ ID NO: 15, and an LCDR3 comprising the amino acid sequence set forth in SEQ ID NO: 16.

In certain aspects of this disclosure, the heavy chain variable region comprises an HCDR1 comprising or the amino acid sequence set forth in SEQ ID NO:5, an HCDR2 comprising the amino acid sequence set forth in SEQ ID NO: 6, and an HCDR3 comprising the amino acid sequence set forth in SEQ ID NO: 7; and the light chain variable region comprises an LCDR1 comprising the amino acid sequence set forth in SEQ ID NO: 8, an LCDR2 comprising the amino acid sequence set forth in SEQ ID NO: 9, and an LCDR3 comprising the amino acid sequence set forth in SEQ ID NO: 10.

In certain aspects of this disclosure, the amino acid sequence the heavy chain of the antibody has at least about 85%, 90%, 91%, 92%, 93%, 94%, 95%, 96%, 97%, 98%, or 99% sequence identity to the amino acid sequence selected from the group consisting of SEQ ID NO: 21.

In certain aspects of this disclosure, the amino acid sequence of the heavy chain of the antibody has least 85%, 90%, 91%, 92%, 93%, 94%, 95%, 96%, 97%, 98%, or 99% sequence identity to the amino acid sequence selected from the group consisting of SEQ ID NO: 22.

In certain aspects of this disclosure, the amino acid sequence the light chain of the antibody has at least about 85%, 90%, 91%, 92%, 93%, 94%, 95%, 96%, 97%, 98%, or 99% sequence identity to the amino acid sequence selected from the group consisting of SEQ ID NO: 23.

In certain aspects of this disclosure, the amino acid sequence of the light chain of the antibody has least 85%, 90%, 91%, 92%, 93%, 94%, 95%, 96%, 97%, 98%, or 99% sequence identity to the amino acid sequence selected from the group consisting of SEQ ID NO: 24.

In certain aspects of this disclosure, the heavy chain variable region comprises the amino acid sequence selected from the group consisting of SEQ ID NOs: 1 and 2.

In certain aspects of this disclosure, the light chain variable region is encoded by a nucleic acid sequence selected from the group consisting of SEQ ID NOs: 3 and 4.

In certain aspects of this disclosure, the heavy chain variable region comprises a sequence that has at least 98%, or 99% sequence identity to the sequence set forth in SEQ ID NO: 1 and the light chain variable region comprises a sequence that has at least 90% sequence identity to the sequence set forth in SEQ ID NO: 3.

In certain aspects of this disclosure, the heavy chain comprises a sequence that has at least 90% sequence identity to the sequence set forth in SEQ ID NO: 2 and the light chain variable region comprises a sequence that has at least 90% sequence identity to the sequence set forth in SEQ ID NO: 4.

In certain aspects of this disclosure, the dissociation constant (K_{D}) for binding to human GRP78 is less than 1.25×10⁻⁹ M. In certain aspects of this disclosure, the dissociation constant (K_{D}) for binding to human GRP78 is between 8×10⁻¹⁰ M and 1.1×10⁻⁹ M. In certain aspects of this disclosure, the dissociation constant (K_{D}) is determined by BIAcore® analysis.

In certain aspects of this disclosure, the Tm1 of the thermal unfolding curve of the antibody or antigen-binding fragment is greater than 60° C.

In certain aspects of this disclosure, the Tm1 of the antibody or antigen-binding fragment is between about 60° C. and about 70° C.

In certain aspects of this disclosure, the Tm1 of the antibody or antigen-binding fragment is about 64° C., about 65° C., about 66° C., about 67° C., about 68° C., or about 69° C.

In certain aspects of this disclosure, the melting temperature profile is monophasic.

In certain aspects of this disclosure, the onset temperature of aggregation (Tagg) is increased greater than about 1° C., greater than about 2° C., greater than about 3° C., greater than about 4° C. or greater than about 5° C. compared to a reference antibody, wherein the reference antibody is B4 or an antibody comprising a variable heavy chain set forth in SEQ ID NO: 1 and a variable light chain set forth in SEQ ID NO: 3, optionally wherein the reference antibody is of the same form.

In certain aspects of this disclosure, the onset temperature of aggregation (Tagg) is increased greater than about 1° C., greater than about 2° C., greater than about 3° C., greater than about 4° C. or greater than about 5° C. compared to a reference antibody, wherein the reference antibody is F6 or an antibody comprising a variable heavy chain set forth in SEQ ID NO: 2 and a variable light chain set forth in SEQ ID NO: 4, optionally wherein the reference antibody is of the same form.

In certain aspects of this disclosure, the onset temperature of aggregation (Tagg) is greater than about 67° C.

In certain aspects of this disclosure, the onset temperature of aggregation (Tagg) is between about 67° C. and about 71° C.

In certain embodiments the Tagg of the antibody or antigen-binding fragment is about 67° C., about 68° C., about 69° C., about 70° C., or about 71° C.

In certain aspects of this disclosure, the antibody or antigen-binding fragment binds selectively to cell surface human GRP78.

In certain embodiments disclosed herein, the antibody or fragment binds human GRP78 expressed on the surface of cells with an EC50 of less than or equal to 0.020 μg/mL optionally wherein the EC50 is determined by flow cytometry.

In certain embodiments disclosed herein, the antibody or fragment binds human GRP78 expressed on the surface of cells with an EC50 of between about 0.010 μg/mL and 0.020 μg/mL, optionally wherein the EC50 is determined by flow cytometry.

In certain aspects of this disclosure, the antibody or fragment binds human GRP78 expressed on the surface of cells, with an EC50 of about 0.015 μg/mL, about 0.016 μg/mL, about 0.017 μg/mL, about 0.018 μg/mL, about 0.019 μg/mL, or about 0.020 μg/mL, optionally wherein the EC50 is determined by flow cytometry.

In certain aspects of this disclosure, the antibody or antigen-binding fragment is internalized by human GRP78-expressing cells.

In certain aspects, this disclosure includes a nucleic acid encoding the heavy chain of the antibody or antigen-binding fragment thereof of any one of the above aspects or any other aspect or embodiment disclosed herein.

In certain aspects, this disclosure includes a nucleic acid encoding the heavy chain and the light chain of the antibody or antigen-binding fragment thereof of any one of the above aspects or any other aspect or embodiments disclosed herein.

In certain aspects, this disclosure includes a vector comprising the nucleic acid of any one of the above aspects or any other aspect or embodiments disclosed herein. In certain aspects of this disclosure, the vector is an expression vector.

In certain aspects, this disclosure includes a vector comprising a nucleic acid encoding the heavy chain and a nucleic acid encoding the light chain, wherein the heavy chain and light chain is of the antibody or antigen-binding fragment of any one of the above aspects or any other aspect or embodiments disclosed herein.

In certain aspects of this disclosure, the vector is a bicistronic vector.

In certain aspects, this disclosure includes a vector system, comprising a first vector comprising a first nucleic acid encoding a heavy chain and a second vector comprising a second nucleic acid encoding a light chain, wherein the heavy chain and the light chain is of the antibody or antigen-binding fragment of any one of the above aspects or any other aspect or embodiments disclosed herein.

In certain aspects of this disclosure, the first vector and the second vector are each an expression vector.

In certain aspects, this disclosure includes a host cell comprising the vector of any one of the above aspects or any other aspect or embodiments disclosed herein.

In certain aspects of this disclosure, the host cell is a mammalian cell.

In certain aspects of this disclosure, the host cell is a human cell.

In certain aspects, this disclosure includes an immunoconjugate having the formula Ab-(L-D)x, with the terms “Ab”, “L” and “D” as defined herein.

In certain aspects of this disclosure, Ab of the formula Ab-(L-D)x is an antibody or antigen-binding fragment thereof that selectively binds to GRP78, such as the antibodies disclosed herein. In certain aspects of this disclosure, Ab is an antibody that specifically binds to an epitope of GRP78, the Ab comprising the heavy chain set forth in SEQ ID NO:1 and the light chain set forth in SEQ ID NO:3. In certain aspects of this disclosure, Ab is an antibody that specifically binds to an epitope of GRP78, the Ab comprising the heavy chain set forth in SEQ ID NO:2 and the light chain set forth in SEQ ID NO:4. In aspects of this disclosure, Ab is an antibody that specifically binds to an epitope of GRP78, the Ab comprising the sequence set forth in SEQ ID NO:1 and SEQ ID NO:3 (B4). In aspects of this disclosure, Ab is an antibody that specifically binds to an epitope of GRP78, the Ab comprising the sequence set forth in SEQ ID NO:2 and SEQ ID NO: 4 (F6).

In certain aspects of this disclosure, L is a linker. Linkers (“L”) and linker drug constructs “L-D” as used herein are in some cases presented as linker reagents, i.e., linker reagents have reactive moieties which can react with, and covalently bond to, an appropriate atom or atoms of the antibody or antigen binding fragment. Ab-(L-D)x and Ab-L-D refer to an antibody drug conjugate (ADC) where one or more reactive linker moieties have reacted with and formed one or more covalent bonds with an atom or atoms of an antibody or antigen-binding fragment.

In certain aspects of this disclosure, the linker L contains a cleavable linker moiety.

In certain embodiments of the above aspects or any other aspect or embodiment disclosed herein of this disclosure, the cleavable linker moiety is a tetrapeptide.

In certain embodiments of the above aspects or any other aspect or embodiment disclosed herein of this disclosure, the tetrapeptide cleavable linker moiety is glycine-glycine-phenylalanine-glycine (“GGFG”).

In certain aspects of this disclosure, the linker L is a cathepsin-cleavable linker moiety.

In certain embodiments of the above aspects or any other aspect or embodiment disclosed herein of this disclosure, the cathepsin-cleavable linker moiety comprises a valine-citrulline (Val-Cit).

In certain aspects of this disclosure, the linker L comprises a self-immolative linker, see, for example, Edupuganti, V et al. Recent Patents on Anti-Cancer Drug Discovery 16.4 (2021): 479-497.) In certain embodiments of the above aspects or any other aspect or embodiment disclosed herein of this disclosure, the self-immolative linker moiety comprises p-aminobenzyloxycarbonyl (PAB).

In certain embodiments of the above aspects or any other aspect or embodiment disclosed herein of this disclosure, the linker also includes a polyethylene glycol (“PEG”) chain.

In certain embodiments of the above aspects or any other aspect or embodiment disclosed herein of this disclosure, the PEG chain is composed of from 24 to 6 polyethylene glycol (—CH₂—CH₂—O—) units.

In certain aspects of this disclosure, the linker L comprises two or more of the aspects or embodiments described herein.

In certain aspects of this disclosure, the linker reagent is MC-Val-Cit-PAB, which incorporates the MC (maleimidocaproyl) reactive moiety:

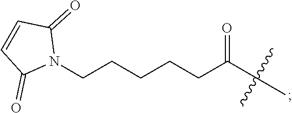 [SMILES: *C(=O)CCCCCN1C(=O)C=CC1=O]

the Val-Cit (valine-citrulline) cleavable moiety:

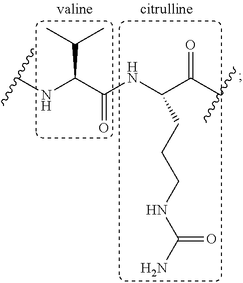 [SMILES: *N[C@H](C(=O)N[C@@H](CCCNC(N)=O)C(*)=O)C(C)C]

and the PAB (p-aminobenzyloxycarbonyl) moiety:

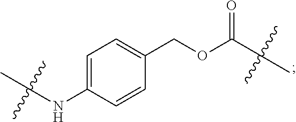 [SMILES: *Nc1ccc(COC(*)=O)cc1]

to form the linker reagent MC-Val-Cit-PAB having the structure:

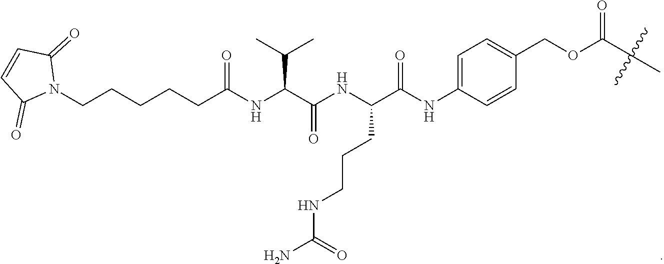 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1]

wherein the frilled line indicates the attachment point of the cytotoxic drug D.

In certain aspects of this disclosure, the linker reagent is TBR-Glu-(Val-Cit-PAB)-amino-PEG₂₄-Me, having the structure:

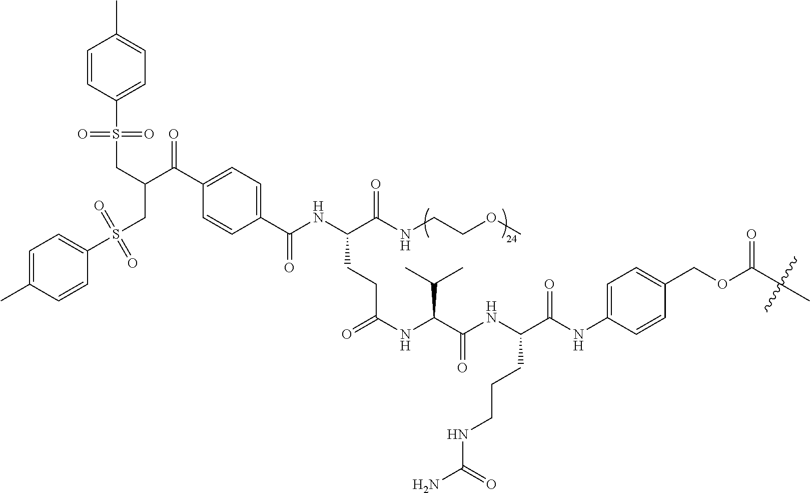

wherein the frilled line indicates the attachment point of the cytotoxic drug D.

In certain aspects of this disclosure, the linker reagent is TBR-[Glu-(Val-Cit-PAB)]₂-Glu-(amino-PEG₂₄-Me)₂, having the structure:

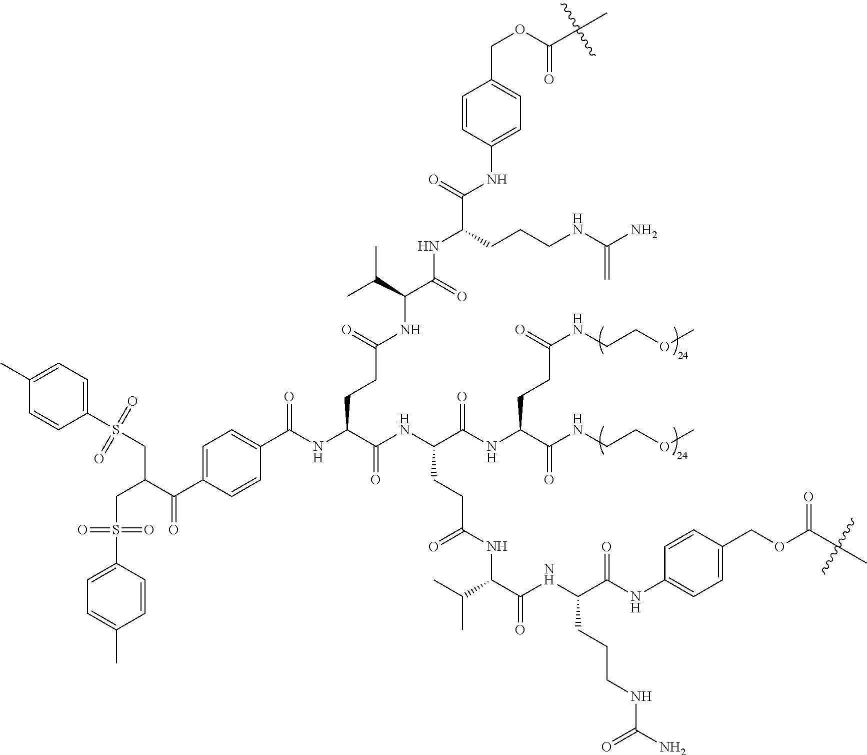

wherein the frilled lines indicate the attachment point of the cytotoxic drug D.

In certain aspects of this disclosure, the linker is a pH cleavable linker.

In certain aspects of this disclosure, the linker reagent is CL2A, having the structure:

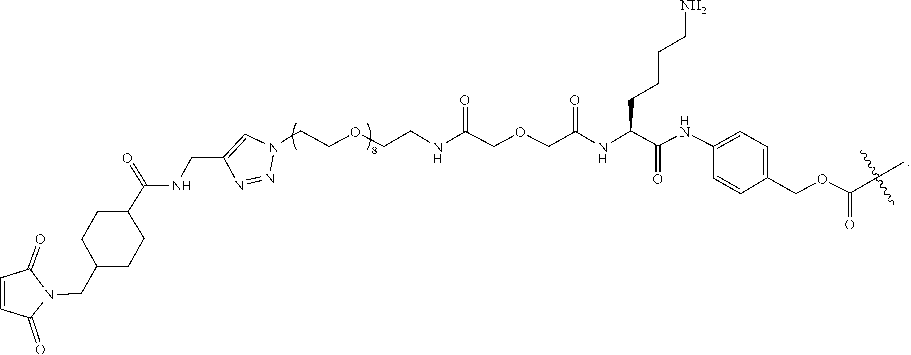

wherein the frilled line indicates the attachment point of the cytotoxic drug D.

In an embodiment of the of the above aspects or any other aspect or embodiment disclosed herein, the cytotoxic drug D is a topoisomerase inhibitor.

In certain aspects of this disclosure, the topoisomerase inhibitor is a camptothecin analog.

In an embodiment of the above aspects or any other aspect or embodiment disclosed herein, the cytotoxic camptothecin analog is Exatecan.

In an embodiment of the above aspects or any other aspect or embodiment disclosed herein, the cytotoxic camptothecin analog is an Exatecan derivative. In specific embodiments, the Exatecan derivative is one of those disclosed in U.S. Pat. No. 5,834,476.

In an embodiment of the above aspects or any other aspect or embodiment disclosed herein, the cytotoxic camptothecin analog is DXd.

In an embodiment of the above aspects or any other aspect or embodiment disclosed herein, the cytotoxic camptothecin analog is SN-38:

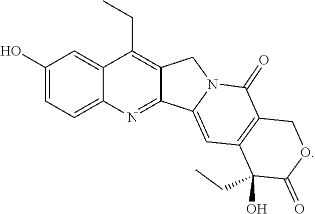 [SMILES: CCc1c2c(nc3ccc(O)cc13)-c1cc3c(c(=O)n1C2)COC(=O)[C@]3(O)CC]

In certain aspects, the cytotoxic drug D is an auristatin or an auristatin analog.

In an embodiment of the above aspects or any other aspect or embodiment disclosed herein, the cytotoxic auristatin is MMAE, having the structure:

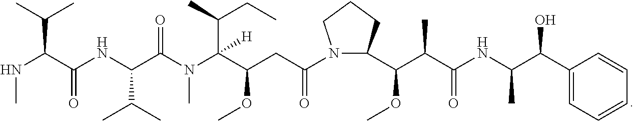 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In certain aspects of this disclosure, the L-D reagent is MC-Val-Cit-PAB-MMAE, having the structure:

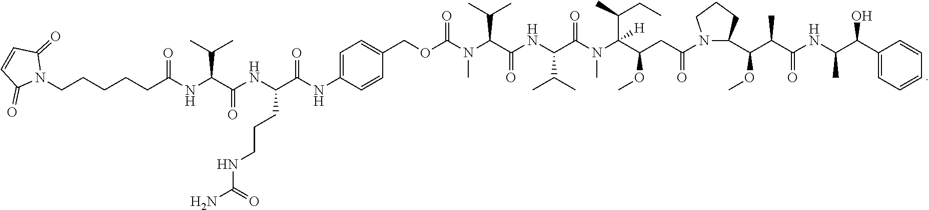 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

In certain aspects of this disclosure, the L-D reagent is TBR-Glu-(Val-Cit-PAB-MMAE)-amino-PEG₂₄-Me, having the structure:

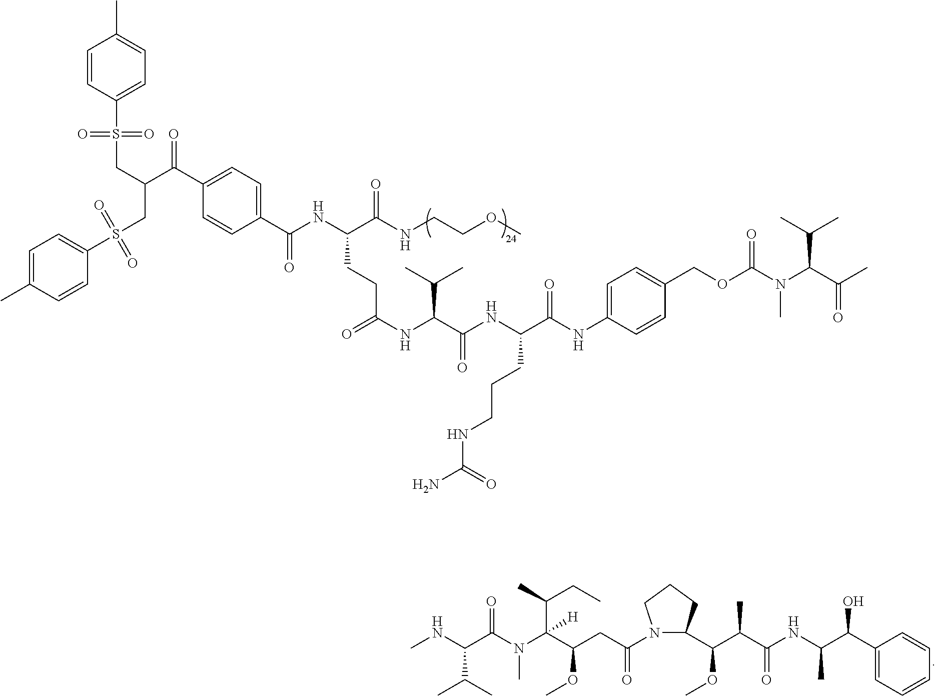

In certain aspects of this disclosure, the L-D reagent is CL2A-SN38, having the structure:

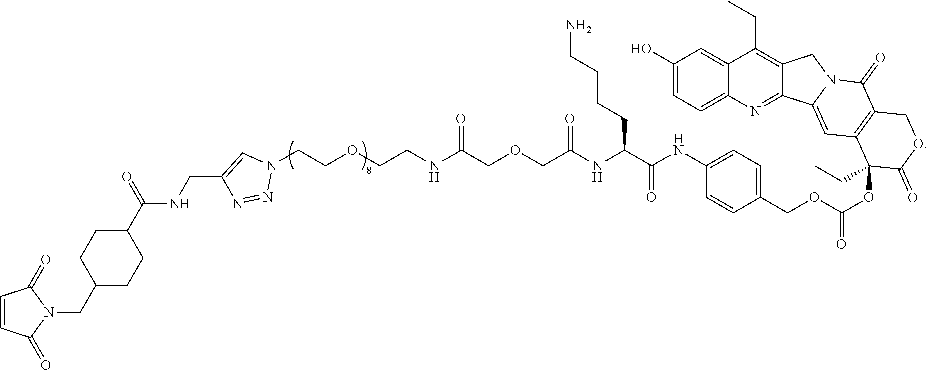

In certain aspects of this disclosure, the L-D reagent is TBR-[Glu-(Val-Cit-PAB-SN38)]₂-Glu-(amino-PEG₂₄-Me)₂, having the structure:

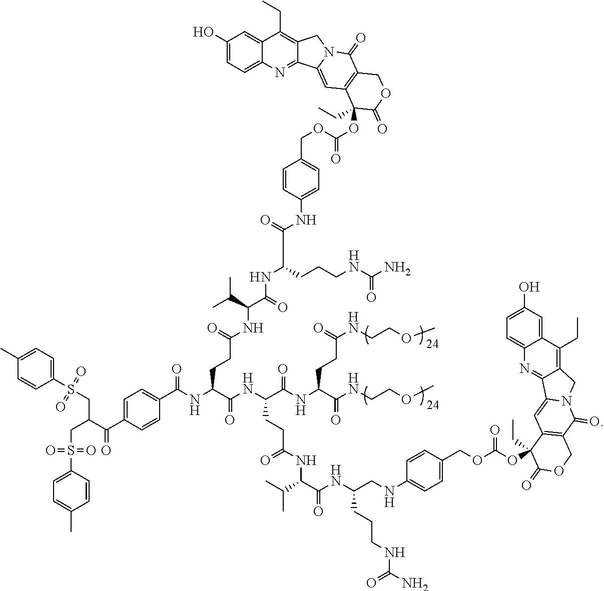

In certain embodiments of the above aspects or any other aspect or embodiment disclosed herein, the L-D reagent is TBLR-GGFG-DXd having the structure:

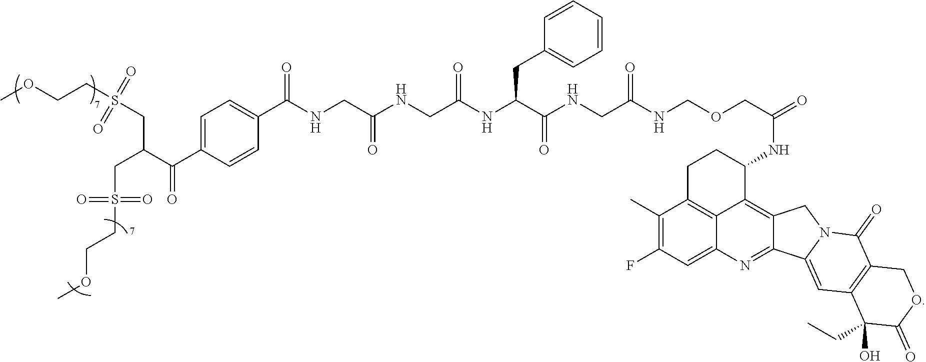

In certain embodiments of the above aspects or any other aspect or embodiment disclosed herein, the L-D reagent is TBLR-Glu-(GGFG-DXd)₂ having the structure:

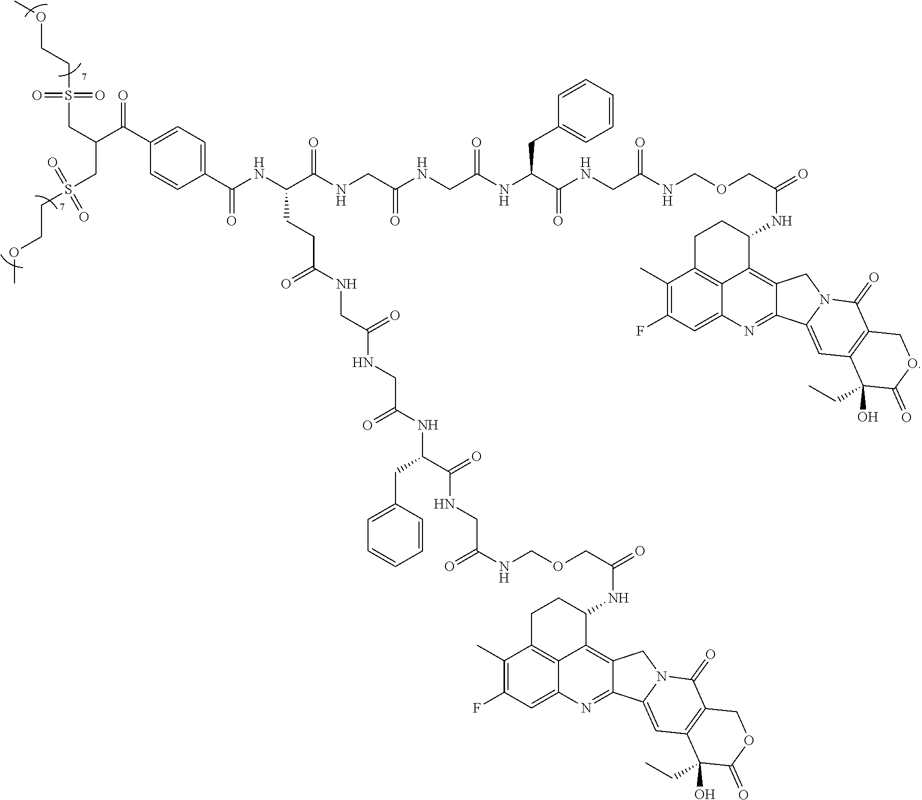

In certain aspects, this disclosure includes an immunoconjugate having the formula Ab-(L-D)x.

In certain aspects of this disclosure, the drug to antibody ratio (“DAR”) is or is from about 1 to about 8.

In certain embodiments of the above aspects or any other aspect or embodiment disclosed herein of this disclosure, the DAR is or is about 4.

In certain embodiments of the above aspects or any other aspect or embodiment disclosed herein of this disclosure, the DAR is or is about 8.

In certain aspects, this disclosure includes a pharmaceutical composition comprising the antibody or antigen-binding fragment of any one of the above aspects or any aspect or embodiment disclosed herein and a pharmaceutically acceptable carrier.

In certain aspects, this disclosure includes a pharmaceutical composition comprising the immunoconjugate of any one of the above aspects or any aspect or embodiment disclosed herein and a pharmaceutically acceptable carrier.

In certain aspects, this disclosure includes a method for treating, ameliorating, and/or preventing cancer in a subject in need thereof, the method comprising administering to the subject an effective amount of the antibody or antigen-binding fragment thereof of the above aspects or any aspect or embodiment disclosed herein.

In certain aspects, this disclosure includes a method for treating, ameliorating, and/or preventing cancer in a subject in need thereof, the method comprising administering to the subject an effect amount of the immunoconjugate of any one of the above aspects or any aspect or embodiment disclosed herein.

In certain aspects, this disclosure includes a method for treating, ameliorating, and/or preventing cancer in a subject in need thereof, the method comprising administering to the subject an effect amount of the pharmaceutical composition of the above aspects or any aspect or embodiment disclosed herein.

In certain aspects, this disclosure includes a method for treating, ameliorating, and/or preventing cancer in a subject in need thereof, the method comprising administering to the subject an effect amount of the pharmaceutical composition of the above aspects or any aspect or embodiment disclosed herein.

In certain aspects, this disclosure includes a method for treating, ameliorating, and/or preventing a condition associated with expression/overexpression of GRP78 in a subject in need thereof, the method comprising administering to the subject an effective amount of the antibody or antigen-binding fragment thereof of the above aspects or any aspect or embodiment disclosed herein.

In certain aspects, this disclosure includes a method for treating, ameliorating, and/or preventing a condition associated with expression/overexpression of GRP78 in a subject in need thereof, the method comprising administering to the subject an effect amount of the immunoconjugate of any one of the above aspects or any aspect or embodiment disclosed herein.

In certain aspects, this disclosure includes a method for treating, ameliorating, and/or preventing a condition associated with expression/overexpression of GRP78 in a subject in need thereof, the method comprising administering to the subject an effect amount of the pharmaceutical composition of the above aspects or any aspect or embodiment disclosed herein.

In certain aspects, this disclosure includes a method for treating, ameliorating, and/or preventing a condition associated with expression/overexpression of GRP78 in a subject in need thereof, the method comprising administering to the subject an effect amount of the pharmaceutical composition of the above aspects or any aspect or embodiment disclosed herein.

In certain aspects, the condition associated with expression/overexpression of GRP78 is systemic lupus erythematosus and other autoimmune diseases, obesity, cardiovascular disease, diabetes, inflammatory bowel diseases such as Crohn's and ulcerative colitis, and/or benign prostatic hyperplasia (BPH).

In certain aspects, this disclosure includes a method for inducing tumor regression in a subject, the method comprising administering to the subject an effective amount of the antibody or antigen-binding fragment thereof of any one of the above aspects or any aspect or embodiment disclosed herein.

In certain aspects, this disclosure includes a method for inducing tumor regression in a subject in need thereof, the method comprising administering to the subject an effective amount of the immunoconjugate of any one of the above aspects or any aspect or embodiment disclosed herein.

In certain aspects, this disclosure includes a method for inducing tumor regression in a subject in need thereof, the method comprising administering to the subject an effective amount of the pharmaceutical composition of the above aspects or any aspect or embodiment disclosed herein.

In certain aspects, this disclosure includes a method for inducing tumor regression in a subject in need thereof, the method comprising administering to the subject an effect amount of the pharmaceutical composition of the above aspects or any aspect or embodiment disclosed herein.

In certain aspects of this disclosure, the cancer is associated with expression GRP78. In certain aspects of this disclosure, the tumor is associated with expression of GRP78. In certain aspects of this disclosure, the cancer is a tumor. In certain aspects of this disclosure, the cancer is a blood cancer (liquid tumor).

In certain aspects of this disclosure, the GRP78 is differentially (e.g., to a greater extent) expressed on the cancer cells compared to normal cells.

### BRIEF DESCRIPTION OF THE DRAWINGS

The following detailed description of preferred embodiments of this disclosure will be better understood when read in conjunction with the appended figures. For the purpose of illustrating this disclosure, there are shown in the drawings embodiments which are presently preferred. It should be understood, however, that this disclosure is not limited to the precise arrangements and instrumentalities of the embodiments shown in the drawings.

FIG. 1 illustrates results of tumor regression studies that were performed using female NSG™ mice implanted with the breast cancer xenograft model TM00098 (BR1126F) and compares responses to ADCs comprising antibody B4 (see FIG. 1A) and antibody F6 (see FIG. 1B); both of which were conjugated to linker reagent TBR-Glu-(Val-Cit-PAB-MMAE)-amino-PEG₂₄-Me (B4-TB-Glu-(Val-Cit-PAB-MMAE)-amino-PEG₂₄-Me and F6-TB-Glu-(Val-Cit-PAB-MMAE)-amino-PEG₂₄-Me) at doses of 1 mg/kg, 2 mg/kg, 4 mg/kg and saline placebo.

FIG. 2 (FIG. 2A for B4; FIG. 2B for F6) presents scatter plots and averages of tumor weights for each mouse treated as described in FIG. 1.

FIG. 3 illustrates results of tumor regression studies that were performed using female NSG™ mice implanted with the breast cancer xenograft model TM00098 (BR1126F) and compares responses to ADCs comprising antibody B4 (see FIG. 3A) and antibody F6 (see FIG. 3B); both of which were conjugated to TBR-Glu-(Val-Cit-PAB-Exatecan)-amino-PEG₂₄-Me (B4-TB-Glu-(Val-Cit-PAB-Exatecan)-amino-PEG₂₄-Me and F6-TB-Glu-(Val-Cit-PAB-Exatecan)-amino-PEG₂₄-Me) at doses of 1 mg/kg, 2 mg/kg, 4 mg/kg and saline placebo.

FIG. 4 (FIG. 4A for B4; FIG. 4B for F6) presents scatter plots and averages of tumor weights for each mouse treated as described in FIG. 3.

FIG. 5 illustrates results of tumor regression studies that were performed using female NSG™ mice implanted with the breast cancer xenograft model TM00098 (BR1126F) and compares responses to ADCs comprising antibody B4 (see FIG. 5A) and antibody F6 (see FIG. 5B); each of which were conjugated to TBLR-GGFG-DXd (B4-TB-GGFG-DXd and F6-TB-GGFG-DXd) at doses of 1 mg/kg, 2 mg/kg, 4 mg/kg and saline placebo.

FIG. 6 (FIG. 6A for B4; FIG. 6B for F6) presents scatter plots and averages of tumor weights for each mouse treated as described in FIG. 5.

FIG. 7 illustrates results of tumor regression studies that were performed using mice implanted with prostate cancer xenograft model MDA-PCa-118b and shows dose response to ADCs comprising antibody B4 conjugated to TBR-Glu-(Val-Cit-PAB-MMAE)-amino-PEG₂₄-Me (B4-TB-Glu-(Val-Cit-PAB-MMAE)-amino-PEG₂₄-Me) at doses of 1 mg/kg, 2 mg/kg, 4 mg/kg and saline placebo.

FIG. 8 illustrates results of tumor regression studies that were performed using mice implanted with prostate cancer xenograft model MDA-PCa-118b and shows dose response to ADCs comprising antibody B4 conjugated to TBLR-GGFG-DXd (B4-TB-GGFG-DXd) at doses of 1 mg/kg, 2 mg/kg, 4 mg/kg and saline placebo.

FIG. 9 illustrates results of tumor regression studies that were performed using mice implanted with prostate cancer xenograft model MDA-PCa-118b and shows dose response to ADCs comprising antibody B4 conjugated to TBR-[Glu-(GGFG-DXd)]₂-Glu-(amino-PEG₂₄-Me)₂(4) (B4-TB-[Glu-(GGFG-DXd)]₂-Glu-(amino-PEG₂₄-Me)₂) at doses of 1 mg/kg, 2 mg/kg, 4 mg/kg and saline placebo.

FIG. 10 illustrates the chemical structure of an ADC with four of the TBR-Glu-(Val-Cit-PAB-MMAE)-amino-PEG₂₄-Me copies bonded to the disulfide group of a GRP78 antibody: (Ab)-TB-Glu-(Val-Cit-PAB-MMAE)-amino-PEG₂₄-Me. Variable x is typically from about 1 to about 8 and is often about 4.

FIG. 11 illustrates the chemical structure of an ADC with four of the TBR-Glu-(Val-Cit-PAB-Exatecan)-amino-PEG₂₄-Me copies bonded to the disulfide group of a GRP78 antibody: (Ab)-TB-Glu-(Val-Cit-PAB-Exatecan)-amino-PEG₂₄-Me. Variable x is typically from about 1 to about 8 and is often about 4.

FIG. 12 illustrates the chemical structure of an ADC with four of the TBLR-GGFG-DXd copies bonded to the disulfide group of a GRP78 antibody: B4-TB-GGFG-DXd). Variable x is typically from about 1 to about 8 and is often about 4.

FIG. 13 illustrates the chemical structure of an ADC with four of the TBR-[Glu-(GGFG-DXd)]₂-Glu-(amino-PEG₂₄-Me)₂ copies bonded to a disulfide group of a GRP78 antibody: (Ab)-TB-[Glu-(GGFG-DXd)]₂-Glu-(amino-PEG₂₄-Me)₂. Variable x is typically from about 1 to about 8 and is often about 4.

FIG. 14 illustrates results of tumor regression studies that were performed using mice implanted with prostate cancer xenograft model DU145 (see Example 10) and shows dose response to B4-TB-GGFG-DXd ADCs at doses of 10 mg/kg, 15 mg/kg, 20 mg/kg and PBS (phosphate buffered saline) placebo, with two doses administered at each of days 0 and 7.

FIG. 15 illustrates results of tumor regression studies that were performed using mice implanted with prostate cancer xenograft model DU145 (see Example 10) and shows dose response to B4-TB-GGFG-DXd ADCs at doses of 10 mg/kg, 15 mg/kg, 20 mg/kg and PBS (phosphate buffered saline) placebo, with one dose administered at each of days 0, 4, 7 and 11.

FIG. 16 illustrates results of tumor regression studies that were performed using mice implanted with colorectal cancer (CRC) xenograft model TM000375 (see Example 10) and shows dose response to B4-TB-GGFG-DXd ADCs at doses of 5 mg/kg and 10 mg/kg as compared to irinotecan at 15 mg/kg and PBS (phosphate buffered saline) placebo, administered weekly.

FIG. 17 illustrates results of tumor regression studies that were performed using mice implanted with colorectal cancer (CRC) xenograft model TM000980 (see Example 10) and shows dose response to B4-TB-GGFG-DXd ADCs at doses of 5 mg/kg and 10 mg/kg as compared to irinotecan at 15 mg/kg and PBS (phosphate buffered saline) placebo, administered weekly.

### DETAILED DESCRIPTION

### Definitions

Unless defined otherwise, all technical and scientific terms used herein have the same meaning as commonly understood by one of ordinary skill in the art to which this disclosure pertains. Although any methods and materials similar or equivalent to those described herein can be used in the practice for testing of the present disclosure, the preferred materials and methods are described herein. In describing and claiming the present disclosure, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

Three letter standard abbreviations for the amino acids are used herein with the accepted notations as described in Ryadnov, Maxim, and Ferenc Hudecz, eds. Amino Acids, Peptides and Proteins: Volume 42. Royal Society of Chemistry, 2017. pp. x-xvii among other sources and can be summarized in pertinent part as follows:

The three-letter symbol standing alone represents the unmodified intact amino acid, of the L-configuration unless otherwise stated (but the L-configuration may also be indicated for emphasis: e.g. L-Ala). The same three-letter symbol, however, can also stand for the corresponding amino acid residue in a peptide. The symbols can thus be used to represent peptides (e.g. AlaAla or Ala-Ala=alanylalanine). When nothing is shown attached to either side of the three-letter symbol it is meant to be understood that the amino group (always understood to be on the left) or carboxyl group is unmodified, but this can be emphasized, so AlaAla=H-AlaAla-OH. Side chains are understood to be unsubstituted if nothing is shown, but a substituent can be indicated by use of brackets or attachment by a vertical bond up or down. Thus, an O-methylserine residue may be shown as 1, 2, or 3 in the schematic below:

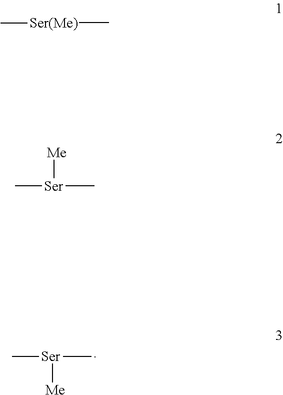

Similarly, this disclosure may use single-letter codes for amino acids stands as an alternate shorthand for representing the twenty standard amino acids. The comprehensive list of these single-letter codes, along with their respective amino acids, includes: A: Alanine; C: Cysteine; D: Aspartic acid; E: Glutamic acid; F: Phenylalanine; G: Glycine; H: Histidine; I: Isoleucine; K: Lysine; L: Leucine; M: Methionine; N: Asparagine; P: Proline; Q: Glutamine; R: Arginine; S: Serine; T: Threonine; V: Valine; W: Tryptophan and Y: Tyrosine. See also Table 1 herein.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “recombinant,” as used herein, refers to nucleic acid or protein molecules formed by artificial (e.g. laboratory-derived) methods of genetic engineering (e.g. molecular cloning) that bring together genetic sequences from multiple sources, thus creating sequences that would not otherwise be found in natural genomes.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. IgG antibodies are typically tetramers of immunoglobulin molecules comprising two heavy chain and two light chain polypeptides. Each polypeptide chain contains three complementarity-determining regions (CDRs), which bind to the antigen and define the antibody's antigen specificity.

As used herein, the term “antibody” and “antibodies” can also include polypeptides or polypeptide complexes derived from full-length antibodies. These polypeptide complexes may be naturally occurring or constructed from single chain antibodies or antibody fragments and retain an antigen-specific binding ability. The antibodies of the present disclosure may exist in a variety of forms including, for example, polyclonal antibodies, monoclonal antibodies, Fv, Fab and F(ab′)₂, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). “Antibody” also includes single-domain antibodies, such as camelid antibodies (Riechmann, 1999, Journal of Immunological Methods 231:25-38), composed of either a VL or a VH domain which exhibit sufficient affinity for the target, and multispecific antibodies formed from antibody fragments. The antibody fragment also includes a human antibody or a humanized antibody or a portion of a human antibody or a humanized antibody.

The term “antibody fragment” refers to a polypeptide comprising or derived from a portion of an intact antibody and may comprise the antigen-binding fragment of an intact antibody.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

The term “specifically binds” to an antigen or epitope is a term that is well understood in the art, and methods to determine such specific binding are also well known in the art. A binding molecule, such as an antibody or antigen-binding fragment is said to exhibit “specific binding,” to include “preferential binding,” if it reacts or associates more frequently, more rapidly, with greater duration and/or with greater affinity with a particular target than it does with alternative targets. An antibody specifically binds or preferentially binds to a target if it binds with greater affinity, avidity, more readily, and/or with greater duration than it binds to other substances. For example, an antibody that specifically or preferentially binds to GRP78 is an antibody that binds GRP78 with greater affinity, avidity, more readily, and/or with greater duration than it binds to a non-target protein epitope. It is also understood by reading this definition that; for example, a binding molecule that specifically or preferentially binds to a first target may or may not specifically or preferentially bind to a second target. It is also understood by reading this definition that specific binding or preferential binding does not necessarily require (although it can and does include) exclusive binding. Methods to determine such specific or preferential binding are also well known in the art, e.g., an immunoassay. The term “specifically binds,” as used herein, can also be meant an antibody, or a ligand, which recognizes and binds with a cognate binding partner (e.g., a stimulatory and/or costimulatory molecule present on a T cell) protein or peptide present in a sample, but which antibody or ligand does not substantially recognize or bind other molecules or regions in the sample.

The term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “antibody-drug conjugate” or “ADC” as used herein refers to an antibody-based molecular complex comprising an antibody or antigen-binding polypeptide fragment derived from an antibody conjugated to a biologically active drug molecule, often referred to as the “payload”. ADC-associated drug molecules are often cytotoxic in function, which allows the ADC complex to kill cells expressing specific molecular targets, especially tumor cells.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of this disclosure in prevention of the occurrence of tumor in the first place.

The term “Kd”, “K_{D}” or “equilibrium dissociation constant,” as used herein, refers to a calculated, quantitative measurement of an antibody's affinity for its epitope. The K_{D} represents a ratio of the rate of binding (Kon) and dissociation (Koff) between the antibody and its epitope. K_{D} and affinity are inversely related in that a high affinity interaction is characterized by a low K_{D}.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, gastric (stomach) cancer, testicular cancer, bone cancer, esophageal cancer, thyroid cancer, to name some of the more common types of cancer, but also includes less common and rare forms of cancer.

The phrase “condition associated with expression/overexpression of GRP78” refers any cancer or non-cancer indication associated with the expression or overexpression of GRP78. Exemplary cancer indications are recited in the previous paragraph. Exemplary non-cancer indications include systemic lupus erythematosus and other autoimmune diseases, obesity, cardiovascular disease, diabetes, inflammatory bowel diseases such as Crohn's and ulcerative colitis, and benign prostatic hyperplasia (BPH), among other conditions.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of this disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Exemplary conservative substitutions are shown below in Table 1.

TABLE 1

| | Exemplary Conservative | Preferred Conservative |
|---|---|---|
| Original Amino | Substitutions of the Original | Substitution of the Original |
| Acid Residues in | Amino Acid Residues of | Amino Acid Residues of |
| SEQ ID NOS. 1-24 | SEQ ID NOS. 1-24 | SEQ ID NOS. 1-24 |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4 Diamino-butyric Acid, Gln, Asn | Arg |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

Thus, one or more amino acid residues within the CDR regions of an antibody of this disclosure can be replaced with other amino acid residues from the same side chain family (preferably as shown in Table 1) and the altered antibody can be tested for the ability to bind epitopes such as GRP78 using the functional assays described herein.

The term “dysregulated” when used in the context of the level of expression or activity of GRP78 refers to the level of expression, activity, or plasma-membrane localization that is different from the expression level, activity or plasma-membrane localization of GRP78 in an otherwise identical healthy animal, organism, tissue, cell or component thereof. In certain embodiments, the dysregulation of expression or activity is associated with a disease state including cancer in which expression level, activity or plasma-membrane localization of GRP78 is dysregulated within cancer cells.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” and “chimeric” forms of non-human (e.g., murine) antibodies are immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized and chimeric antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized and chimeric antibodies can comprise residues which are found neither in the recipient antibody nor in the imported CDR or framework sequences. These modifications are made to further refine and optimize antibody performance. In general, the humanized and chimeric antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized and chimeric antibody optimally will also comprise at least a portion of an immunoglobulin constant region (Fc), typically that of a human immunoglobulin. The World Health Organization (WHO) International Nonproprietary Name (INN) Expert Group has defined requirements for non-human derived antibodies to be considered “humanized”. According to guidelines, comparison of a candidate antibody to human sequences should be done through the International Immunogenetics Information System® (IMGT®) DomainGapAlign tool (www.imgt.org). This tool interrogates the IMGT® database of antibody germline variable region genes where the alignment score is made only against germline sequence variable region exons, thus omitting part of CDR3 and the J region from the analysis. For an antibody to be “humanized”, in addition to being “closer to human than to other species”, the top “hit” should be human and the identity to human sequences must be at least 85%, otherwise the antibody would be designated as “chimeric”. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine. In certain embodiments, a nucleic acid molecule encoding one or more binding agents described herein may be inserted into one or more expression vectors, as discussed below in greater detail. In such embodiments, the binding agent may be encoded by nucleotides corresponding to the amino acid sequence. The particular combinations of nucleotides (codons) that encode the various amino acids (AA) are well known in the art, as described in various references used by those skilled in the art (e.g., Lewin, B. Genes V, Oxford University Press, 1994). The nucleotide sequences encoding the amino acids of said binding agents may be ascertained with reference to Table 2, for example. Nucleic acid variants may use any combination of nucleotides that encode the binding agent.

TABLE 2
Codons Encoding Amino Acids (AA) of SEQ ID NOS. 1-24 or Variants Thereof

| AA | Codon | AA | Codons | AA | Codons | AA | Codons |
|---|---|---|---|---|---|---|---|
| Phe (F) | TTT | Ser (S) | TCT | Tyr (Y) | TAT | Cys (C) | TGT |
| | TTC | | TCC | | TAC | | TGC |
| Leu (L) | TTA | | TCA | TERM | TAA | TERM | TGA |
| | TTG | | TCG | | TAG | Trp (W) | TGG |
| | CTT | Pro (P) | CCT | His (H) | CAT | Arg (R) | CGT |
| | CTC | | CCC | | CAC | | CGC |
| | CTA | | CCA | Gln (Q) | CAA | | CGA |
| | CTG | | CCG | | CAG | | CGG |
| Ile (I) | ATT | Thr (T) | ACT | Asn (N) | AAT | Ser (S) | AGT |
| | ATC | | ACC | | AAC | | AGC |
| | ATA | | ACA | Lys (K) | AAA | Arg (R) | AGA |
| Met (M) | ATG | | ACG | | AAG | | AGG |
| Val (V) | GTT | Ala (A) | GCT | Asp (D) | GAT | Gly (G) | GGT |
| | GTC | | GCC | | GAC | | GGC |
| | GTA | | GCA | Glu (E) | GAA | | GGA |
| | GTG | | GCG | | GAG | | GGG |

Those of ordinary skill in the art understand that a nucleotide sequence encoding a particular amino acid sequence may be easily derived from the amino acid sequences of SEQ ID NOS: 1-24. For instance, it may be deduced from the amino acid sequence of B4 Variable Heavy Chain CDR1 (SGGYY; SEQ ID NO: 5) and the information presented in Table 2 that the amino acid sequence may be encoded by the nucleotide sequence AGCGGCGGATATTATTGGTCC (SEQ ID NO: 26) (see, e.g., the tool provided at https://www.bioinformatics.org/sms2/rev_trans.html). Those of ordinary skill in the art would understand that nucleotide sequences encoding any of SEQ ID NOS. 2-24 may be deduced in the same way, and such nucleotide sequences are contemplated herein. Where the binding agents are antibodies, nucleotide sequences encoding the variable regions thereof may also be isolated from the phage and/or hybridoma cells expressing the same cloned into expression vectors to produce antibody preparations.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

“Single chain antibodies” refer to antibodies formed by recombinant DNA techniques in which immunoglobulin heavy and light chain fragments are linked to each other using an engineered span of amino acids to recapitulate the Fv region of an antibody as a single polypeptide. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “ThioBridge linker” or “TBR” or “TB®” as used herein means 4-(3-tosyl-2-(tosylmethyl)propanoyl)benzoic acid:

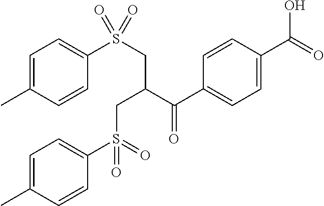 [SMILES: Cc1ccc(S(=O)(=O)CC(CS(=O)(=O)c2ccc(C)cc2)C(=O)c2ccc(C(=O)O)cc2)cc1]

and its derivatives. A ThioBridge linker, optionally when combined with other linker moieties, may be a component of a linker or a linker reagent.

The term “ThioBridge linear linker” or “TBLR” or “TBL®” as used herein means 4-[3-(Me-(PEG)₇)sulfonyl-2-[(Me-(PEG)₇))sulfonylmethyl]propanoyl]benzoic acid:

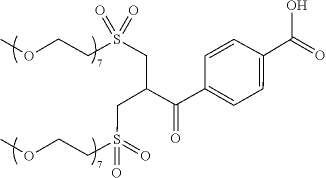

and its derivatives, optionally when combined with other linker moieties, may be a component of a linker or a linker reagent.

In the production of linker-drug moieties (also sometimes referred to as linker-payload moieties) TBR and TBLR are often interchangeable, or at least alternatives to one another, as a skilled chemist would understand how to synthesize a TBR version of a TBLR linker-drug moiety, or a TBLR version of a TBR linker-drug moiety. Moreover, it is noted that conjugates made using linker-drug comprising TBR are identical to conjugates made using TBLR, and vice versa, in that the distinguishing features of TRB and TBLR are no longer present post-conjugation.

The term “treating” as used herein can refer to medical interventions performed with the purpose of ameliorating or preventing a disease state. The term refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof. The Kabat and Clothia numbering systems may also be used to determine the position of other features of antibodies or antibody-derived fragments such as framework regions (FRs) and the like.

The term “thermal melting point” or “thermal melting temperature”, often abbreviated “Tm” or “Tmelt”, when used herein refers to the temperature at which a protein undergoes denaturation, an unfolding process in which quaternary, tertiary, and secondary structure are lost. The Tm of a protein can be measured directly via observing changes in intrinsic fluorescence due to tryptophan and tyrosine residues. Such Tm assays based on fluorescence can be used to rank the stability of protein constructs or compare different formulations to optimize various conditions. Other methods of measuring Tm are known in the art, and include but are not limited to static light scattering (SLS), dynamic light scattering (DLS), differential scanning calorimetry, circular dichromism, and the like and may be used to determine the Tm of the antibodies and antibody-derived fragments of the current disclosure.

The term “thermal aggregation” or “thermal aggregation temperature”, often abbreviated “Tagg” as used herein refers to the temperature at which protein molecules in suspension begin to oligomerize or aggregate. The temperature at which Tagg occurs depends on the physical properties of the protein itself and by the composition of the buffer solution.

Ranges: throughout this disclosure, various aspects of this disclosure can be presented in a range format. It should be understood that the description in range format is merely for convenience and brevity and should not be construed as an inflexible limitation on the scope of this disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Provided herein are binding molecules, such as antibodies (including antigen-binding antibody fragments and immunoconjugates (e.g. antibody drug conjugates) that bind GRP78 and can be used to target GRP78 for treating, ameliorating, and/or preventing a disease or condition, such as a cancer or other non-cancerous diseases or conditions. Also provided are nucleic acid molecules encoding the antibodies. Also provided are methods of making and using the antibodies and antigen-binding fragments as well as immunoconjugates (e.g. antibody drug conjugates) containing the antibodies and antigen-binding fragments. Also provided are compositions, including pharmaceutical compositions, containing such antibodies or antigen-binding fragments or immunoconjugates (e.g. antibody drug conjugates) containing the antibodies or antigen-binding fragments. In some aspects, the provided compositions, antibodies or antigen-binding fragments or immunoconjugates (e.g. antibody drug conjugates) containing the antibodies or antigen-binding fragments can be used in connection with a therapy or a method of treatment, amelioration, and/or prevention.

The present disclosure is based on the observation that humanized antibodies or antibody derived antigen-binding fragments specific for GRP78 can be used to treat, ameliorate, and/or prevent cancers and other diseases associated with the expression of GRP78, typically in the form of csGRP78 (cell surface GRP78). The antibodies of this disclosure can also be used in antibody drug conjugates useful for the same. Also provided are methods and compositions comprising the GRP78 specific antibodies or antigen-binding fragments of this disclosure for the treatment, amelioration, and/or prevention of cancers and other conditions.

Glucose Response Protein 78, also referred to GRP78, is a chaperone heat shock protein involved in the unfolded protein response (UBR) in the Endoplasmic Reticulum (ER) in normal cells. Under stress, the overexpression of GRP78 on the cell membrane mediates the response in the vast amount of disordered proteins. Initial evidence has shown that GRP78 is closely associated with the progression and poor prognosis of various cancers, including lung cancer, prostate cancer, colorectal cancer, and non-cancerous indications such as systemic lupus erythematosus and other autoimmune diseases, obesity, cardiovascular disease, diabetes, inflammatory bowel diseases such as Crohn's and ulcerative colitis, and benign prostatic hyperplasia (BPH). (Via S et al., J Transl Med. 2021; 19: 118; Arap M et al., Can Cell 2004; 6: 275; Song W et al., Eur J Pharmacol. 2025; DOI: 10.1016/j.ejphar.2025.177′308; Matsueda Y et al., Lupus Sci Med. 2018; 5: e000281; Pan D. et al., Int J Environ Res Public Health 2022; 19: 15965; Crane E et al., JCI1 Insight 2018; 3: e99363; Nourbakhsh M et al., J Endocrinol Invest. 2022; 45:649; Long Y et al., Eur J Histochem, 2022; 66: 3415; Fu X et al., Cell Death Dis. 2022; 13: 78.)

The provided antibodies (e.g. the antibodies and antigen-binding fragments thereof of this disclosure), as well as antibody drug conjugates containing the same, exhibit advantageous features compared to certain existing antibodies directed to GRP78 and immunoconjugates related to the same. In particular, the provided embodiments produce a new antibody-based molecule that retains high affinity target binding for GRP78, accessibility to the target in an in vivo setting, and an ability to internalize upon binding to the target at the cell surface, as desirable features for an antibody-drug conjugate.

Thus, among provided antibodies are antibodies that exhibit one or more, or in some cases, all of the improved features of reduced immunogenicity, higher binding affinity to the GRP78 target, better thermostability and ability to be internalized by an GRP78 plasma membrane-expressing cells. For instance, among provided antibodies that exhibit such features are the antibodies designated F6 and B4. These two antibodies bind to the same region of GRP78 but bind to distinct epitope and secondary amino acids. Critical epitope residues for B4 are R261, H265 and E329, supported by secondary residues K271, K272 and D333. Critical epitope residues for F6 are R261 and R279, supported by secondary residues K113, K268 and K271. In some embodiments, there also are provided antibody drug conjugates (ADCs) containing a provided antibody (e.g. F6 or B4) linked to a cytotoxic drug and uses thereof for treating, ameliorating, and/or preventing GRP78-expressing cancers or other conditions associated with GRP78 expression.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Antibodies and Antigen-Binding Fragments

Provided herein are binding polypeptides that bind to Glucose Response Protein 78 (GRP78), ultimately for use in ADCs as detailed herein. For example, the binding polypeptides and antibodies specifically bind to GRP78. Such binding polypeptides include antibodies (including antigen-binding fragments thereof) that specifically bind to GRP78, such as human GRP78. In provided embodiments, an anti-GRP78 antibody or antigen-binding fragment provided herein is a human antibody or an antigen-binding fragment thereof. In some embodiments, an anti-antibody or antigen-binding fragment thereof provided herein binds GRP78 protein. In some embodiments, the binding polypeptides and antibodies of this disclosure bind to GRP78 with high affinity. Preferably, the binding polypeptides and antibodies of this disclosure specifically recognize naturally expressed GRP78 protein on a cell and do not cross-react, or cross react minimally, to other surface molecules on that cell.

In certain aspects, this disclosure provides an antibody comprising an antigen-binding domain that specifically binds to an epitope of GRP78. In certain embodiments, the antigen-binding domain comprises a heavy chain variable region that comprises three heavy chain complementarity-determining regions (HCDRs) and a light chain variable region that comprises three light chain complementarity-determining regions (LCDRs).

In some embodiments, amino acid residues in variable domain sequences and full-length antibody sequences are numbered according to the Kabat numbering convention. Kabat et al. (1991), “Sequences of Proteins of Immunological Interest,” 5th Ed. Public Health Service, National Institutes of Health, Bethesda, MD. Under the Kabat numbering scheme, in some embodiments, the CDR amino acid residues in the heavy chain variable domain (VH) are numbered 31-35/35A/35B (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Using the Kabat numbering system, the actual linear amino acid sequence of a peptide may contain fewer or additional amino acids corresponding to a shortening of, or insertion into, a framework (FR) or CDR of the variable domain. For example, a heavy chain variable domain may include amino acid insertions (residue 52a, 52b and 52c according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

However, although CDR sequences are exemplified according to the Kabat numbering convention, it will be apparent to those skilled in the art that there are alternative numbering conventions for amino acid residues in variable domain sequences and full-length antibody sequences. For instance, an alternative numbering convention is the Chothia numbering scheme, for example as set out in Chothia et al. (1989) Nature 342:877-883. Under the Chothia numbering scheme, in some embodiments, the CDR amino acids in the VH are numbered 26-32/33/34 (HCDR1), 52-56 (HCDR2), and 95-102 (HCDR3); and the CDR amino acid residues in the light chain variable domain (VL) are numbered 24-34 (LCDR1), 50-56 (LCDR2), and 89-97 (LCDR3). In some examples, amino acid residues in variable domain sequences and full-length antibody sequences are numbered according to a combination of both Chothia and Kabat CDR definitions. In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35/35A/35B (HCDR1), 50-65 (HCDR2), and 95-102 (HCDR3) in a VH, e.g., a mammalian VH, e.g., a humanized VH; and amino acid residues 24-34 (LCDR1), 50-56 (LCDR2), and 89-97 (LCDR3) in a VL, e.g., a mammalian VL, e.g., a humanized VL.

Other numbering conventions for CDR sequences available to a skilled person include, for example, “AbM” (university of Bath) and “contact” (University College London) methods. Databases can be used for CDR identification such as to identify CDRs based on Kabat numbering, Chothia or other numbering scheme. It also is understood that, in some cases, other residues are part of the CDR sequence depending on the particular convention used and factors such as the structure and protein folding of the antibody and would be understood to be so by a skilled person.

In certain aspects, this disclosure provides an antibody or antigen-binding fragment thereof comprising an HCDR1 comprising the amino acid sequence SGGYYWS (SEQ ID NO: 5) or SNSAAWN (SEQ ID NO: 11). Also provided is an isolated binding polypeptide comprising an HCDR2 comprising the amino acid sequences YIYYSGSTYYNPSLES (SEQ ID NO: 6) or RTYYRSKWYNDYAVSVKS (SEQ ID NO: 12). Also provided is an isolated binding polypeptide comprising an HCDR3 comprising the amino acid sequence YSSIDAFEI (SEQ ID NO: 7) or DPYYYDSSGYYYFAFDI (SEQ ID NO: 13).

Also provided is an isolated binding polypeptide comprising a light chain variable region that comprises an LCDR1 comprising the amino acid sequences GGDDIGSKSVH (SEQ ID NO: 8) or GGDNIGSKSVH (SEQ ID NO: 14). Also provided is an isolated binding polypeptide comprising an LCDR2 comprising the amino acid sequences DDGDRPS (SEQ ID NO: 9) or DDSDRPS (SEQ ID NO: 15). Also provided is an isolated binding polypeptide comprising an LCDR3 comprising the amino acid sequences QVWDSSSDQYV (SEQ ID NO:10) or QVWDSTSHHVV (SEQ ID NO: 16).

In certain aspects, this disclosure provides an antibody comprising an HCDR1 comprising the amino acid sequence SGGYYWS (SEQ ID NO: 5), an HCDR2 comprising the amino acid sequence YIYYSGSTYYNPSLES (SEQ ID NO: 6), an HCDR3 comprising the amino acid sequence YSSIDAFEI (SEQ ID NO: 7), an LCDR1 comprising the amino acid sequence GGDDIGSKSVH (SEQ ID NO: 8), an LCDR2 comprising the amino acid sequence DDGDRPS (SEQ ID NO: 9), and an LCDR3 comprising the amino acid sequence QVWDSSSDQYV (SEQ ID NO:10).

In certain aspects, this disclosure provides an antibody comprising an HCDR1 comprising the amino acid sequence SSNSAAWN (SEQ ID NO: 11), an HCDR2 comprising the amino acid sequence RTYYRSKWYNDYAVSVKS (SEQ ID NO: 12), an HCDR3 comprising the amino acid sequence DPYYYDSSGYYYFAFDI (SEQ ID NO: 13), an LCDR1 comprising the amino acid sequence GGDNIGSKSVH (SEQ ID NO: 14), an LCDR2 comprising the amino acid sequence DDSDRPS (SEQ ID NO: 15), and an LCDR3 comprising the amino acid sequence QVWDSTSHHVV (SEQ ID NO: 16). Tolerable variations of the complementarity determining regions (CDR) sequences will be known to those of skill in the art.

For example, in some embodiments the polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 5-10 or SEQ ID NOs: 11-16. In some preferred embodiments, the polypeptide includes each of SEQ ID NOs. 5-10 (as in antibody B4) or each of SEQ ID Nos. 11-16 (as in antibody F6). In some preferred embodiments, the polypeptide includes each of SEQ ID NOs. 8-10 (as in antibody B4 scFv) or each of SEQ ID Nos. 14-16 (as in antibody F6 scFv).

In some embodiments, the binding polypeptide, such as an anti-GRP78 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 1 (B4 antibody Variable Heavy Chain) and a light chain variable region that has at least 80%, 85%, 90%, 95%, 96%, 96%, 97%, 98%, or 99% sequence identity to the sequence set forth in SEQ ID NO:3 (B4 antibody Variable Light Chain). In some embodiments, the heavy chain variable region is set forth in SEQ ID NO: 1 and the light chain variable region is set forth in SEQ ID NO:3. In some embodiments, the antibody is antibody B4 or an antigen-binding fragment thereof.

In some embodiments, the binding polypeptide, such as an anti-GRP78 antibody or antigen-binding fragment, comprises a heavy chain variable region with an amino acid sequence that has at least 80%, 85%, 90%, 95%, 96%, 96%, 97%, 98%, or 99% sequence identity to the sequence set forth in SEQ ID NO: 2 (F6 antibody Variable Heavy Chain) and a light chain variable region that has at least 80%, 85%, 90%, 95%, 96%, 96%, 97%, 98%, or 99% sequence identity to the sequence set forth in SEQ ID NO: 4 (F6 antibody Variable Light Chain). In some embodiments, the heavy chain variable region is set forth in SEQ ID NO: 2 and the light chain variable region is set forth in SEQ ID NO: 4. In some embodiments, the antibody is antibody F6 or an antigen-binding fragment thereof.

In some embodiments, the binding polypeptide, such as an anti-GRP78 antibody or antigen-binding fragment, comprises a region at least 80%, 85%, 90%, 95%, 96%, 96%, 97%, 98%, or 99% sequence identity to the sequence set forth in SEQ ID NO: 19 (B4 ScFv). In some embodiments, the binding polypeptide, such as an anti-GRP78 antibody or antigen-binding fragment, comprises a region at least 80%, 85%, 90%, 95%, 96%, 96%, 97%, 98%, or 99% sequence identity to the sequence set forth in SEQ ID NO: 20 (F6 ScFv).

An antibody of this disclosure can be prepared using an antibody having one or more of the VH and/or VL sequences disclosed herein as a starting material to engineer a modified antibody, which modified antibody may have altered properties as compared with the starting antibody. An antibody can be engineered by modifying one or more amino acids within one or both variable regions (i.e., VH and/or VL), for example within one or more CDR regions and/or within one or more framework regions. Additionally, or alternatively, an antibody can be engineered by modifying residues within the constant region(s), for example to alter the effector function(s) of the antibody.

In some embodiments, the binding polypeptide is an antibody or an antigen-binding fragment thereof.

Among the provided antibodies are antibody fragments. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region. In some embodiments, the antigen-binding fragment is selected from the group consisting of a Fab, a single-chain variable fragment (scFv), a single-domain antibody, and a nanobody. In further embodiments, the antibody is a full-length antibody. In provided embodiments, the antibody or antigen-binding fragment is a humanized antibody or an antigen-binding fragment thereof.

In some embodiments, the antibody or antigen-binding fragment thereof may contain at least a portion of an immunoglobulin constant region, such as one or more constant region domain. In some embodiments, the constant region of the heavy chain includes at least the heavy chain constant region 1 (CH1) and the constant region of the light chain includes the light chain constant region. In some embodiments, the constant region of the heavy chain further includes a hinge domain, a CH2 and/or CH3 domain. In some embodiments, the heavy chain constant domain is from a constant chain, or a portion thereof containing the CH1, hinge, CH2 and/or CH3, of a human IgG, such as a human IgG1 or IgG4. In some embodiments, the light chain constant domain is from a constant chain of a human kappa light or lambda light chain.

Among the provided antibodies are full-length antibodies containing a heavy chain with any one of the heavy chain variable regions provided herein combined with a human heavy chain constant region; and a light chain with any one of the light chain variable regions provided herein combined with a human light chain constant region. In some embodiments, the constant region of the heavy chain is a human IgG1 heavy chain constant region. In some embodiments, the heavy chain constant region is set forth in SEQ ID NO: 17 or a sequence that has at least 85%, 86%, 87%, 88%, 89%, 90%, 91%, 92%, 93%, 94%, 95%, 96%, 97%, 98%, or 99% sequence identity to SEQ ID NO: 17. In some embodiments, the heavy chain constant region is set forth in SEQ ID NO: 17. In some embodiments, the constant region of the light chain is a human kappa light chain constant region. In some embodiments, the light chain constant region is set forth in SEQ ID NO: 18 or a sequence that has at least 85%, 86%, 87%, 88%, 89%, 90%, 91%, 92%, 93%, 94%, 95%, 96%, 97%, 98%, or 99% sequence identity to SEQ ID NO: 18. In some embodiments, the heavy chain constant region is set forth in SEQ ID NO:18. Any of the heavy chain variable regions provided herein may be combined with a suitable human constant region. Any of the light chain variable regions may be combined with a suitable human light chain constant region.

Provided is an ADC comprising an anti-GRP78 antibody comprising a full-length heavy chain comprising the amino acid sequence set forth in SEQ ID NO: 1 and a light chain comprising the amino acid sequence set forth in SEQ ID NO: 3 (e.g., the B4 antibody).

Provided is an ADC comprising an anti-GRP78 antibody comprising a full-length heavy chain comprising the amino acid sequence set forth in SEQ ID NO: 2 and a full-length light chain comprising the amino acid sequence set forth in SEQ ID NO: 4 (e.g., the F6 antibody).

In some embodiments, the provided anti-GRP78 antibody is a full-length antibody comprising (i) at least 85%, 90%, 91%, 92%, 93%, 94%, 95%, 96%, 97%, 98%, or 99% sequence identity to the amino acid sequence set forth in SEQ ID NO: 21; and (ii) at least 85%, 90%, 91%, 92%, 93%, 94%, 95%, 96%, 97%, 98%, or 99% sequence identity to the amino acid sequence set forth in SEQ ID NO: 23.

In some embodiments, the provided anti-GRP78 antibody is a full-length antibody comprising (i) at least 85%, 90%, 91%, 92%, 93%, 94%, 95%, 96%, 97%, 98%, or 99% sequence identity to the amino acid sequence set forth in SEQ ID NO: 22; and (ii) at least 85%, 90%, 91%, 92%, 93%, 94%, 95%, 96%, 97%, 98%, or 99% sequence identity to the amino acid sequence set forth in SEQ ID NO: 24.

In certain embodiments, this disclosure includes an antibody that binds to the same epitope on GRP78 as an antibody of this disclosure (i.e., antibodies that have the ability to cross-compete for binding to GRP78 with any of the antibodies of this disclosure). In a preferred embodiment, the reference antibody or antibody fragment for cross-competition studies can be one of the antibodies or antibody fragments described herein, as comparisons are made using equivalent molecules (e.g. a Fab to a Fab, full-length antibody to a full-length antibody, etc.). For example, BIAcore® analysis, ELISA assays or flow cytometry may be used to demonstrate cross-competition with the antibodies of the current disclosure. The ability of a test antibody to inhibit the binding of, for example B4 and F6, to GRP78 demonstrates that the test antibody can compete with B4 or F6 for binding to GRP78 and thus is considered to bind to the same epitope of GRP78 as B4 and F6.

In some embodiments, any one of the provided anti-GRP78 antibodies are isolated, purified or semi-purified such that they retain specificity in the desired application. Proteins may be recombinant, or synthesized in vitro, although in most embodiments, the proteins are recombinant. Alternatively, a non-recombinant or recombinant protein may be isolated from bacteria.

The polypeptides (preferably antibodies) of this disclosure can include any one or more of the peptides and/or polypeptides having the human amino acid sequences of SEQ ID NOs: 1-24 as shown in Table 3. Preferred combinations of the various peptides and/or polypeptides are discussed herein and include those peptides and/or polypeptides exhibiting binding activity towards human GRP78 (SEQ ID NO: 25).

TABLE 3
Antibody Amino Acid Sequences and Human GRP78

| SEQ ID NO: | Name | Type | Sequence |
|---|---|---|---|
| 1 | B4 Variable | PRT | QVRLQESGPGLVKPSQTLSLTCTVSGGSISSGGYYWS |
| | Heavy Chain | | WIRQHPGKGLEWIGYIY |
| | | | YSGSTYYNPSLESRVTISVDTSKNQFS |
| | | | LKLSSVTAADTAVYYCARYSSIDAFEI |
| | | | WGQGTMVTVSS |
| 2 | F6 Variable | PRT | QVQLQQSGPGLVKPPQTLSLTCAISGDSVSSNSAAWN |
| | Heavy Chain | | WIRQSPSRGLEWLGRTY |
| | | | YRSKWYNDYAVSVKSRITINPDTSKNQ |
| | | | FSLQLNSVTPEDTAVYYCARDPYYYDSSGYYYFDAFD |
| | | | IWGQGTMVTVSS |
| 3 | B4 Variable | PRT | SYVLTQPPSVSVAPGKTATITCGGDDIGSKSVHWYQQ |
| | Light Chain | | KPGQAPVLVVYDDGDRPSGIPERFSGSNSGNTATLAIS |
| | | | RVEAGDEADYYCQVWDSSSDQYVFGSGTKLTVL |
| 4 | F6 Variable | PRT | SYELTQPHSVSVAPGQTARITCGGDNIGSKSVHWYQQ |
| | Light Chain | | RPGQAPVLVVYDDSDRPSGIPERFSGSNSENTATLTISG |
| | | | VEAGDEADYYCQVWDSTSHHVVFGGGTKLTVL |
| 5 | B4 Variable | PRT | SGGYYWS |
| | Heavy Chain | | |
| | CDR1 | | |
| 6 | B4 Variable | PRT | YIYYSGSTYYNPSLES |
| | Heavy Chain | | |
| | CDR2 | | |
| 7 | B4 Variable | PRT | YSSIDAFEI |
| | Heavy Chain | | |
| | CDR3 | | |
| 8 | B4 Variable | PRT | GGDDIGSKSVH |
| | Light Chain | | |
| | CDR1 | | |
| 9 | B4 Variable | PRT | DDGDRPS |
| | Light Chain | | |
| | CDR2 | | |
| 10 | B4 Variable | PRT | QVWDSSSDQYV |
| | Light Chain | | |
| | CDR3 | | |
| 11 | F6 Variable | PRT | SNSAAWN |
| | Heavy Chain | | |
| | CDR1 | | |
| 12 | F6 Variable | PRT | RTYYRSKWYNDYAVSVKS |
| | Heavy Chain | | |
| | CDR2 | | |
| 13 | F6 Variable | PRT | DPYYYDSSGYYYFDAFDI |
| | Heavy Chain | | |
| | CDR3 | | |
| 14 | F6 Variable | PRT | GGDNIGSKSVH |
| | Light Chain | | |
| | CDR1 | | |
| 15 | F6 Variable | PRT | DDSDRPS |
| | Light Chain | | |
| | CDR2 | | |
| 16 | F6 Variable | PRT | QVWDSTSHHVV |
| | Light Chain | | |
| | CDR3 | | |
| 17 | Heavy Chain | PRT | ASTKGPSVFPLAPSSKSTSGGTAALGCLVKDYFPEPVT |
| | Constant | | VSWNSGALTSGVHTFPAVLQSSGLYSLSSVVTVPSSSL |
| | Domain | | GTQTYICNVNHKPSNTKVDKRVEPKSCDKTHTCPPCP |
| | | | APELLGGPSVFLFPPKPKDTLMISRTPEVTCVVVDVSH |
| | | | EDPEVKFNWYVDGVEVHNAKTKPREEQYNSTYRVVS |
| | | | VLTVLHQDWLNGKEYKCKVSNKALPAPIEKTISKAKG |
| | | | QPREPQVYTLPPSREEMTKNQVSLTCLVKGFYPSDIAV |
| | | | EWESNGQPENNYKTTPPVLDSDGSFFLYSKLTVDKSR |
| | | | WQQGNVFSCSVMHEALHNHYTQKSLSLSPGK |
| 18 | Light Chain | PRT | GQPKAAPSVTLFPPSSEELQANKATLVCLISDFYPGAV |
| | Constant | | TVAWKADSSPVKAGVETTTPSKQSNNKYAASSYLSLT |
| | Domain | | PEQWKSHRSYSCQVTHEGSTVEKTVAPTECS |
| 19 | B4 ScFv | PRT | SYVLTQPPSVSVAPGKTATITCGGDDIGSKSVHWYQQ |
| | | | KPGQAPVLVVYDDGDRPSGIPERFSGSNSGNTATLAIS |
| | | | RVEAGDEADYYCQVWDSSSDQYVFGSGTKLTVLSGG |
| | | | STITSYNVYYTKLSSSGTQVRLQESGPGLVKPSQTLSLT |
| | | | CTVSGGSISSGGYYWSWIRQHPGKGLEWIGYIYYSGST |
| | | | YYNPSLESRVTISVDTSKNQFSLKLSSVTAADTAVYYC |
| | | | ARYSSIDAFEIWGQGTMVTVSS |
| 20 | F6 ScFv | PRT | SYELTQPHSVSVAPGQTARITCGGDNIGSKSVHWYQQ |
| | | | RPGQAPVLVVYDDSDRPSGIPERFSGSNSENTATLTISG |
| | | | VEAGDEADYYCQVWDSTSHHVVFGGGTKLTVLSGGS |
| | | | TITSYNVYYTKLSSSGTQVQLQQSGPGLVKPPQTLSLT |
| | | | CAISGDSVSSNSAAWNWIRQSPSRGLEWLGRTYYRSK |
| | | | WYNDYAVSVKSRITINPDTSKNQFSLQLNSVTPEDTAV |
| | | | YYCARDPYYYDSSGYYYFDAFDIWGQGTMVTVSS |
| 21 | B4 Heavy | PRT | MGWSCIILFLVATATGVHSQVRLQESGPGLVKPSQTLS |
| | Chain | | LTCTVSGGSISSGGYYWSWIRQHPGKGLEWIGYIYYSG |
| | Antibody | | STYYNPSLESRVTISVDTSKNQFSLKLSSVTAADTAVY |
| | Sequence | | YCARYSSIDAFEIWGQGTMVTVSSASTKGPSVFPLAPS |
| | | | SKSTSGGTAALGCLVKDYFPEPVTVSWNSGALTSGVH |
| | | | TFPAVLQSSGLYSLSSVVTVPSSSLGTQTYICNVNHKPS |
| | | | NTKVDKRVEPKSCDKTHTCPPCPAPELLGGPSVFLFPP |
| | | | KPKDTLMISRTPEVTCVVVDVSHEDPEVKFNWYVDG |
| | | | VEVHNAKTKPREEQYNSTYRVVSVLTVLHQDWLNGK |
| | | | EYKCKVSNKALPAPIEKTISKAKGQPREPQVYTLPPSR |
| | | | EEMTKNQVSLTCLVKGFYPSDIAVEWESNGQPENNYK |
| | | | TTPPVLDSDGSFFLYSKLTVDKSRWQQGNVFSCSVMH |
| | | | EALHNHYTQKSLSLSPGK |
| 22 | F6 Heavy | PRT | MGWSCIILFLVATATGVHSQVQLQQSGPGLVKPPQTL |
| | Chain | | SLTCAISGDSVSSNSAAWNWIRQSPSRGLEWLGRTYY |
| | Antibody | | RSKWYNDYAVSVKSRITINPDTSKNQFSLQLNSVTPED |
| | Sequence | | TAVYYCARDPYYYDSSGYYYFDAFDIWGQGTMVTVS |
| | | | SASTKGPSVFPLAPSSKSTSGGTAALGCLVKDYFPEPV |
| | | | TVSWNSGALTSGVHTFPAVLQSSGLYSLSSVVTVPSSS |
| | | | LGTQTYICNVNHKPSNTKVDKRVEPKSCDKTHTCPPC |
| | | | PAPELLGGPSVFLFPPKPKDTLMISRTPEVTCVVVDVS |
| | | | HEDPEVKFNWYVDGVEVHNAKTKPREEQYNSTYRVV |
| | | | SVLTVLHQDWLNGKEYKCKVSNKALPAPIEKTISKAK |
| | | | GQPREPQVYTLPPSREEMTKNQVSLTCLVKGFYPSDIA |
| | | | VEWESNGQPENNYKTTPPVLDSDGSFFLYSKLTVDKS |
| | | | RWQQGNVFSCSVMHEALHNHYTQKSLSLSPGK |
| 23 | B4 Light | PRT | MGWSCIILFLVATATGVHSSYVLTQPPSVSVAPGKTAT |
| | Chain | | ITCGGDDIGSKSVHWYQQKPGQAPVLVVYDDGDRPS |
| | Antibody | | GIPERFSGSNSGNTATLAISRVEAGDEADYYCQVWDSS |
| | Sequence | | SDQYVFGSGTKLTVLGQPKAAPSVTLFPPSSEELQANK |
| | | | ATLVCLISDFYPGAVTVAWKADSSPVKAGVETTTPSK |
| | | | QSNNKYAASSYLSLTPEQWKSHRSYSCQVTHEGSTVE |
| | | | KTVAPTECS |
| 24 | F6 Light | PRT | MGWSCIILFLVATATGVHSSYELTQPHSVSVAPGQTAR |
| | Chain | | ITCGGDNIGSKSVHWYQQRPGQAPVLVVYDDSDRPSG |
| | Antibody | | IPERFSGSNSENTATLTISGVEAGDEADYYCQVWDSTS |
| | Sequence | | HHVVFGGGTKLTVLGQPKAAPSVTLFPPSSEELQANK |
| | | | ATLVCLISDFYPGAVTVAWKADSSPVKAGVETTTPSK |
| | | | QSNNKYAASSYLSLTPEQWKSHRSYSCQVTHEGSTVE |
| | | | KTVAPTECS |
| 25 | Human | PRT | MKLSLVAAMLLLLSAARAEEEDKKEDVGTVVGIDLG |
| | GRP78 | | TTYSCVGVFKNGRVEIIANDQGNRITPSYVAFTPEGER |
| | | | LIGDAAKNQLTSNPENTVFDAKRLIGRTWNDPSVQQD |
| | | | IKFLPFKVVEKKTKPYIQVDIGGGQTKTFAPEEISAMVL |
| | | | TKMKETAEAYLGKKVTHAVVTVPAYFNDAQRQATK |
| | | | DAGTIAGLNVMRIINEPTAAAIAYGLDKREGEKNILVF |
| | | | DLGGGTFDVSLLTIDNGVFEVVATNGDTHLGGEDFDQ |
| | | | RVMEHFIKLYKKKTGKDVRKDNRAVQKLRREVEKAK |
| | | | RALSSQHQARIEIESFYEGEDFSETLTRAKFEELNMDLF |
| | | | RSTMKPVQKVLEDSDLKKSDIDEIVLVGGSTRIPKIQQ |
| | | | LVKEFFNGKEPSRGINPDEAVAYGAAVQAGVLSGDQD |
| | | | TGDLVLLDVCPLTLGIETVGGVMTKLIPRNTVVPTKKS |
| | | | QIFSTASDNQPTVTIKVYEGERPLTKDNHLLGTFDLTGI |
| | | | PPAPRGVPQIEVTFEIDVNGILRVTAEDKGTGNKNKITI |
| | | | TNDQNRLTPEEIERMVNDAEKFAEEDKKLKERIDTRN |
| | | | ELESYAYSLKNQIGDKEKLGGKLSSEDKETMEKAVEE |
| | | | KIEWLESHQDADIEDFKAKKKELEEIVQPIISKLYGSAG |
| | | | PPPTGEEDTAEKDEL |

In preferred embodiments, the antibody of this disclosure comprises: a variable heavy chain (VH) comprising complementarity determining regions (CDRs) 1, 2, and 3 as set forth in SEQ ID NOS: 5, 6 and 7, respectively; and a variable light chain (VL) comprising CDRs 1, 2, and 3 as set forth in SEQ ID NOS: 8, 9 and 10, respectively (e.g., in preferred embodiments antibody B4 or B4ScFv (SEQ ID NO: 19)); and/or, a VH comprising SEQ ID NO: 1; and a VL comprising SEQ ID NO: 3 (e.g., in preferred embodiments antibody B4); or, a VH comprising complementarity determining regions (CDRs) 1, 2, and 3 as set forth in SEQ ID NOS: 11, 12 and 13, respectively; and a VL comprising CDRs 1, 2, and 3 as set forth in SEQ ID NOS: 14, 15 and 16, respectively (e.g., in preferred embodiments antibody F6); and/or, a VH comprising SEQ ID NO: 2; and a VL comprising SEQ ID NO: 4 (e.g., in preferred embodiments antibody F6).

In preferred embodiments, the antibody may include SEQ ID NO: 17 as the heavy chain constant region and/or SEQ ID NO: 18 as the light chain constant region. In some preferred embodiments, the amino acid sequence of the heavy chain and light chain of a B4 antibody can be SEQ ID NO: 21 and SEQ ID NO: 23, respectively. In some preferred embodiments, the amino acid sequence of the heavy chain and light chain of a F6 antibody can be SEQ ID NO: 22 and SEQ ID NO: 24, respectively. In some preferred embodiments, the antibody can be the single chain variable fragment of SEQ ID NO: 19 (B4 ScFv) or SEQ ID NO: 20 (F6 ScFv). The amino acid sequences of any of SEQ ID Nos. 1-24 can be substituted/replaced by another amino acid, either conservatively or non-conservatively (for preferred embodiments, see Table 2). As mentioned above, in preferred embodiments, one or more amino acid residues within the CDR regions of an antibody of this disclosure can be replaced with other amino acid residues conservatively (i.e., from the same side chain family) and the altered antibody can be tested for the ability to bind epitopes such as GRP78 using the functional assays described herein. Any substitution that maintains sufficient binding to GRP78, sufficient binding being binding considered acceptable by the user as indicated by the assay (e.g., binds an epitope of human GRP78), is contemplated herein.

Guidance for nucleic acids encoding in humans the amino acids of Table 3 is provided by the nucleic acid sequences of Table 4.

TABLE 4
Antibody Nucleic Acid Sequences

| SEQ ID NO: | Name | Type | Sequence |
|---|---|---|---|
| 26 | B4 | NUCLEIC | AGCGGCGGATATTATTGGTCC |
| | Heavy | ACID | |
| | Chain | | |
| 27 | CDR1 | NUCLEIC | ATGGGATGGTCATGTATCATCCTTTTTCTGGTAGCA |
| | B4 | ACID | ACTGCAACTGGAGTACATAGCCAGGTTAGACTGCA |
| | Heavy | | GGAGTCAGGCCCTGGACTAGTTAAGCCCAGTCAAA |
| | Chain | | CGCTCTCCCTGACCTGCACCGTTAGTGGTGGTTCCA |
| | Antibody | | TTTCTAGCGGCGGATATTATTGGTCCTGGATCAGAC |
| | Sequence | | AGCATCCTGGCAAGGGCCTGGAGTGGATTGGATAC |
| | | | ATCTATTACTCCGGCTCTACATACTACAACCCTAGC |
| | | | TTGGAGAGCAGGGTCACAATCTCTGTGGACACCTC |
| | | | TAAGAATCAGTTTTCCTTGAAGCTGAGCAGTGTCAC |
| | | | CGCTGCGGATACAGCTGTGTACTATTGTGCCCGCTA |
| | | | CTCATCCATCGATGCTTTCGAAATTTGGGGCCAAGG |
| | | | CACCATGGTGACGGTTTCGAGCGCTAGCACCAAGG |
| | | | GCCCATCGGTCTTCCCCCTGGCACCCTCCTCCAAGA |
| | | | GCACCTCTGGGGGCACAGCGGCCCTGGGCTGCCTG |
| | | | GTCAAGGACTACTTCCCCGAACCGGTGACGGTGTC |
| | | | GTGGAACTCAGGCGCCCTGACCAGCGGCGTGCACA |
| | | | CCTTCCCGGCCGTCCTACAGTCCTCAGGACTCTACT |
| | | | CCCTCAGCAGCGTGGTGACCGTGCCCTCCAGCAGC |
| | | | TTGGGCACCCAGACCTACATCTGCAACGTGAATCA |
| | | | CAAGCCCAGCAACACCAAGGTGGACAAGCGGGTTG |
| | | | AGCCCAAATCTTGTGACAAAACTCACACATGCCCA |
| | | | CCGTGCCCAGCACCTGAACTCCTGGGGGGACCGTC |
| | | | AGTCTTCCTCTTCCCCCCAAAACCCAAGGACACCCT |
| | | | CATGATCTCCCGGACCCCTGAGGTCACATGCGTGG |
| | | | TGGTGGACGTGAGCCACGAAGACCCTGAGGTCAAG |
| | | | TTCAACTGGTACGTGGACGGCGTGGAGGTGCATAA |
| | | | TGCCAAGACAAAGCCGCGGGAGGAGCAGTACAAC |
| | | | AGCACGTACCGTGTGGTCAGCGTCCTCACCGTCCTG |
| | | | CACCAGGACTGGCTGAATGGCAAGGAGTACAAGTG |
| | | | CAAGGTCTCCAACAAAGCCCTCCCAGCCCCCATCG |
| | | | AGAAAACCATCTCCAAAGCCAAAGGGCAGCCCCGA |
| | | | GAACCACAGGTGTACACCCTGCCCCCATCCCGGGA |
| | | | GGAGATGACCAAGAACCAGGTCAGCCTGACCTGCC |
| | | | TGGTCAAAGGCTTCTATCCCAGCGACATCGCCGTG |
| | | | GAGTGGGAGAGCAATGGGCAGCCGGAGAACAACT |
| | | | ACAAGACCACGCCTCCCGTGCTGGACTCCGACGGC |
| | | | TCCTTCTTCCTCTACAGCAAGCTCACCGTGGACAAG |
| | | | AGCAGGTGGCAGCAGGGGAACGTCTTCTCATGCTC |
| | | | CGTGATGCATGAGGCTCTGCACAACCACTACACGC |
| | | | AGAAGAGCCTCTCCCTGTCTCCGGGCAAATAATAG |
| 28 | F6 Heavy | NUCLEIC | ATGGGATGGTCATGTATCATCCTTTTTCTGGTAGCA |
| | Chain | ACID | ACTGCAACTGGAGTACATAGCCAGGTGCAGCTTCA |
| | Antibody | | ACAATCAGGCCCTGGCCTCGTTAAACCTCCTCAAA |
| | Sequence | | CACTTTCACTCACATGTGCAATCAGTGGAGACTCCG |
| | | | TGTCTTCCAACTCTGCTGCCTGGAACTGGATCAGGC |
| | | | AGAGCCCCAGTCGAGGTTTGGAATGGCTGGGGAGA |
| | | | ACTTACTACCGTTCTAAGTGGTACAATGATTATGCT |
| | | | GTGTCCGTGAAGAGCCGCATTACAATTAACCCCGA |
| | | | CACCAGCAAGAATCAGTTCTCCCTTCAATTAAACTC |
| | | | TGTTACCCCTGAGGATACTGCTGTGTATTACTGCGC |
| | | | CCGGGACCCTTACTACTATGATAGTTCCGGCTATTA |
| | | | CTACTTCGACGCCTTTGATATCTGGGGCCAAGGTAC |
| | | | CATGGTGACAGTCTCCTCTGCTAGCACCAAGGGCC |
| | | | CATCGGTCTTCCCCCTGGCACCCTCCTCCAAGAGCA |
| | | | CCTCTGGGGGCACAGCGGCCCTGGGCTGCCTGGTC |
| | | | AAGGACTACTTCCCCGAACCGGTGACGGTGTCGTG |
| | | | GAACTCAGGCGCCCTGACCAGCGGCGTGCACACCT |
| | | | TCCCGGCCGTCCTACAGTCCTCAGGACTCTACTCCC |
| | | | TCAGCAGCGTGGTGACCGTGCCCTCCAGCAGCTTG |
| | | | GGCACCCAGACCTACATCTGCAACGTGAATCACAA |
| | | | GCCCAGCAACACCAAGGTGGACAAGCGGGTTGAGC |
| | | | CCAAATCTTGTGACAAAACTCACACATGCCCACCG |
| | | | TGCCCAGCACCTGAACTCCTGGGGGGACCGTCAGT |
| | | | CTTCCTCTTCCCCCCAAAACCCAAGGACACCCTCAT |
| | | | GATCTCCCGGACCCCTGAGGTCACATGCGTGGTGG |
| | | | TGGACGTGAGCCACGAAGACCCTGAGGTCAAGTTC |
| | | | AACTGGTACGTGGACGGCGTGGAGGTGCATAATGC |
| | | | CAAGACAAAGCCGCGGGAGGAGCAGTACAACAGC |
| | | | ACGTACCGTGTGGTCAGCGTCCTCACCGTCCTGCAC |
| | | | CAGGACTGGCTGAATGGCAAGGAGTACAAGTGCAA |
| | | | GGTCTCCAACAAAGCCCTCCCAGCCCCCATCGAGA |
| | | | AAACCATCTCCAAAGCCAAAGGGCAGCCCCGAGAA |
| | | | CCACAGGTGTACACCCTGCCCCCATCCCGGGAGGA |
| | | | GATGACCAAGAACCAGGTCAGCCTGACCTGCCTGG |
| | | | TCAAAGGCTTCTATCCCAGCGACATCGCCGTGGAG |
| | | | TGGGAGAGCAATGGGCAGCCGGAGAACAACTACA |
| | | | AGACCACGCCTCCCGTGCTGGACTCCGACGGCTCC |
| | | | TTCTTCCTCTACAGCAAGCTCACCGTGGACAAGAG |
| | | | CAGGTGGCAGCAGGGGAACGTCTTCTCATGCTCCG |
| | | | TGATGCATGAGGCTCTGCACAACCACTACACGCAG |
| | | | AAGAGCCTCTCCCTGTCTCCGGGCAAATAATAG |
| 29 | B4 Light | NUCLEIC | ATGGGATGGTCATGTATCATCCTTTTTCTGGTAGCA |
| | Chain | ACID | ACTGCAACTGGAGTACATAGCTCCTACGTGCTTAC |
| | Antibody | | GCAACCACCAAGTGTCTCCGTCGCTCCGGGGAAAA |
| | Sequence | | CGGCCACCATCACCTGTGGTGGAGACGACATCGGC |
| | | | TCTAAGTCTGTGCACTGGTATCAACAAAAACCGGG |
| | | | TCAAGCTCCTGTGCTGGTCGTCTATGATGACGGAG |
| | | | ACAGACCTTCCGGCATCCCGGAGAGGTTCAGCGGG |
| | | | TCCAACTCTGGTAACACTGCAACCCTGGCCATTTCC |
| | | | CGGGTTGAAGCCGGGGACGAGGCCGACTATTATTG |
| | | | CCAAGTTTGGGACTCCTCAAGCGACCAATATGTTTT |
| | | | CGGGTCCGGCACCAAGCTGACTGTGTTGGGTCAGC |
| | | | CCAAGGCTGCCCCCTCGGTCACTCTGTTCCCGCCCT |
| | | | CCTCTGAGGAGCTTCAAGCCAACAAGGCCACACTG |
| | | | GTGTGTCTCATAAGTGACTTCTACCCGGGAGCCGTG |
| | | | ACAGTGGCCTGGAAGGCAGATAGCAGCCCCGTCAA |
| | | | GGCGGGAGTGGAGACCACCACACCCTCCAAACAAA |
| | | | GCAACAACAAGTACGCGGCCAGCAGCTACCTGAGC |
| | | | CTGACGCCTGAGCAGTGGAAGTCCCACAGAAGCTA |
| | | | CAGCTGCCAGGTCACGCATGAAGGGAGCACCGTGG |
| | | | AGAAGACAGTGGCCCCTACAGAATGTTCATAATAG |
| 30 | F6 Light | NUCLEIC | ATGGGATGGTCATGTATCATCCTTTTTCTGGTAGCA |
| | Chain | ACID | ACTGCAACTGGAGTACATAGCTCCTATGAGCTAAC |
| | Antibody | | TCAACCTCACTCTGTTTCTGTGGCTCCGGGACAGAC |
| | Sequence | | GGCCCGGATCACCTGTGGCGGCGATAACATCGGCT |
| | | | CCAAGTCTGTCCACTGGTACCAGCAACGACCAGGC |
| | | | CAAGCGCCAGTGCTGGTCGTCTATGATGATAGTGA |
| | | | CAGACCCAGTGGAATACCTGAGCGTTTTAGTGGTT |
| | | | CTAACTCCGAAAACACTGCCACCCTTACCATATCCG |
| | | | GAGTTGAAGCCGGAGACGAGGCAGACTATTACTGC |
| | | | CAAGTCTGGGATTCTACCTCACACCACGTTGTGTTC |
| | | | GGGGGAGGCACCAAGCTGACTGTGTTGGGCCAGCC |
| | | | TAAGGCCGCACCCAGCGTGACCCTGTTCCCTCCAA |
| | | | GTTCTGAGGAACTGCAAGCTAACAAAGCTACACTC |
| | | | GTGTGCCTTATCTCCGACTTTTACCCCGGGGCCGTA |
| | | | ACAGTCGCATGGAAGGCCGATTCTAGCCCAGTCAA |
| | | | AGCCGGAGTGGAGACAACAACCCCTTCCAAGCAGT |
| | | | CAAACAATAAGTATGCCGCTTCCAGTTACCTGTCCC |
| | | | TCACCCCAGAACAGTGGAAAAGCCATCGGTCTTAT |
| | | | TCTTGTCAGGTAACACACGAGGGCAGCACTGTCGA |
| | | | GAAGACCGTTGCTCCCACTGAATGTAGTTAATAG |
| 31 | B4 | NUCLEIC | TACATCTATTACTCCGGCTCTACATACTACAACCCT |
| | Heavy | ACID | AGCTTGGAGAGC |
| | Chain | | |
| | CDR2 | | |
| 32 | B4 | NUCLEIC | TACTCATCCATCGATGCTTTCGAAATT |
| | Heavy | ACID | |
| | Chain | | |
| | CDR3 | | |
| 33 | B4 Light | NUCLEIC | GGTGGAGACGACATCGGCTCTAAGTCTGTGCAC |
| | Chain | ACID | |
| | CDR1 | | |
| 34 | B4 Light | NUCLEIC | GATGACGGAGACAGACCTTCC |
| | Chain | ACID | |
| | CDR2 | | |
| 35 | B4 Light | NUCLEIC | CAAGTTTGGGACTCCTCAAGCGACCAATATGTT |
| | Chain | ACID | |
| | CDR3 | | |

### Exemplary Features

The binding polypeptides, including antibodies and antigen-binding fragments thereof, of this disclosure are characterized by particular functional features or properties of the antibodies.

In some embodiments, the antibodies or antigen-binding fragments thereof specifically bind to csGRP78 protein. In some embodiments, the antibodies or antigen-binding fragments thereof specifically bind to human csGRP78. The observation that an antibody or other binding molecule binds to csGRP78 protein or specifically binds to csGRP78 protein does not necessarily mean that it binds to intracellular GRP78 and/or a GRP78 protein of every species, although preferably the antibody does display cross reactivity with, e.g., rodent and primate species. For example, in some embodiments, features of binding to GRP78 protein, such as the ability to specifically bind thereto and/or to bind with a particular affinity to a particular degree, in some embodiments, refers to the ability with respect to a human GRP78 protein and the antibody may not have this feature with respect to a GRP78 protein of another species, such as mouse.

In some embodiments, the antibodies or antigen-binding fragment thereof, binds, e.g., specifically binds, and/or recognizes, one or more epitopes in GRP78, e.g., human GRP78. In some embodiments, the epitopes are epitopes present on the extracellular domain of GRP78, e.g., human GRP78. In some embodiments, the epitopes include peptide epitopes. In some embodiments, the epitope includes linear epitopes or conformational epitopes or combination thereof.

In some embodiments, the epitope recognized by a provided antibody includes an epitope containing amino acid residues R261, H265, K271, K272, E329, and D333, with reference to numbering of SEQ ID NO: 25. In some embodiments, the epitope recognized by a provided antibody includes an epitope containing amino acid residues R261, H265, K268, K271, E329, and D333, with reference to numbering of SEQ ID NO: 25.

In provided embodiments, the extent of binding of an anti-GRP78 antibody to an unrelated, non-GRP78 protein, such as a non-human GRP78 protein or other non-GRP78 protein, is less than about 10% of the binding of the antibody to human GRP78 protein as measured, e.g., by a radioimmunoassay (RIA).

In some embodiments, the provided antibodies are capable of binding GRP78 protein, such as human GRP78 protein or other GRP78 protein containing an epitope recognized by a provided antibody, with at least a certain affinity, as measured by any of a number of known methods. In some embodiments, the affinity is represented by an equilibrium dissociation constant (K_{D}); in some embodiments, the affinity is represented by EC₅₀.

A variety of assays are known for assessing binding affinity and/or determining whether a binding molecule (e.g., an antibody or fragment thereof) specifically binds to a particular ligand (e.g., an antigen, such as a GRP78 protein). It is within the level of a skilled artisan to determine the binding affinity of a binding molecule, e.g., an antibody, for an antigen, e.g., GRP78, such as human GRP78, such as by using any of a number of binding assays that are well known in the art. For example, in some embodiments, a BIAcore® instrument can be used to determine the binding kinetics and constants of a complex between two proteins (e.g., an antibody or fragment thereof, and an antigen, such as a GRP78 protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed nucleic acids or binding of proteins.

In some embodiments, the binding molecule, e.g., antibody or fragment thereof, binds, such as specifically binds, to an antigen, e.g., a GRP78 protein or an epitope therein, with a binding affinity with a K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 10⁵ M⁻¹. In some embodiments, the antibody or fragment thereof binds, such as specifically binds, to an antigen, e.g., a GRP78 protein or an epitope therein, with a binding affinity with a K_{D}(i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. The on-rate (association rate constant; kₒₙ or kₐ; units of 1/Ms) and the off-rate (dissociation rate constant; k_{off} or k_{d}; units of 1/s) can be determined using any of the assay methods known in the art, for example, surface plasmon resonance (SPR).

In some embodiments, the binding molecule, e.g., antibody or fragment thereof, binds, such as specifically binds, to an antigen, e.g., a GRP78 protein or an epitope therein, with a K_{D} of 1.3×10⁻⁹ M or less. In some embodiments, the dissociation constant (K_{D}) for binding to human GRP78 is less than 1.25×10⁻⁹ M. In some embodiments, the K_{D} is about 1.2×10⁻⁹ M, 1.1×10⁻⁹ M, 1.0×10⁻⁹ M, 9.5×10⁻¹⁰ M, 9.0×10⁻¹⁰ M or 8.5×10⁻¹⁰ M, including any values between any of the foregoing. In some embodiments, the dissociation constant (K_{D}) for binding to human GRP78 is between 8×10⁻¹⁰ M and 1.1×10⁻⁹ M. In some embodiments, the K_{D} is about 1.0 nM±0.1 nM. In particular embodiments, binding affinity is determined using a BIAcore® instrument using standard techniques in the art (e.g., the manufacturer's instructions).

In some embodiments, properties or features of the provided antibodies (e.g., antigen-binding fragments) are described in relation to properties observed for another antibody, e.g., a reference antibody. In some embodiments, the reference antibody is a non-human anti-GRP78 antibody, such as a murine anti-GRP78 antibody.

In some embodiments, among the provided antibodies, such as full-length antibodies or antigen-binding fragments thereof, are antibodies in which the dissociation constant (K_{D}) for binding to human GRP78 is more than 1.2 fold better (e.g. lower K_{D}) than the reference antibody. In some embodiments, the K_{D} for binding to human GRP78 is about 1.2-fold, 1.3-fold, 1.4-fold, 1.5-fold, 1.6-fold, 1.7-fold, 1.8-fold, 1.9-fold, 2.0-fold or better (e.g. lower K_{D}) than the reference antibody.

Among the provided antibodies (e.g., antigen-binding fragments) are those that compete for binding with and/or bind to the same or overlapping epitopes of a GRP78 protein as those bound by the reference antibody described herein but contain a distinct VH and V_{L}region. In some embodiments, among provided antibodies are antibodies that contain distinct set of CDRs, e.g., distinct heavy and light chain CDR1, CDR2, and CDR3.

In some embodiments, the provided binding molecules, such as antibodies or antigen-binding fragments thereof, bind to cell surface GRP78 (csGRP78). In some embodiments, the provided binding molecules, such as antibodies or antigen-binding fragments thereof, display a binding preference for csGRP78-expressing cells as compared to csGRP78-negative cells. In some embodiments, the binding preference is observed where a significantly greater degree of binding is measured to the GRP78-expressing cells, as compared to the non-expressing cells. In some embodiments, the fold change in degree of binding (e.g. EC50) detected, for example, as measured by mean fluorescence intensity in a flow cytometry-based assay and/or dissociation constant or EC₅₀, to the csGRP78-expressing cells as compared to the non-csGRP78-expressing cells, is at least 1.5, 2, 3, 4, 5, 6, or more, and/or is as great, or greater, than the fold change observed for the corresponding form of the reference antibody.

In some embodiments, the provided binding molecules, such as antibodies or antigen-binding fragments, are capable of being internalized by the cell into which it binds. Hence, a provided binding molecule, such as a provided antibody or antigen-binding fragment thereof, is capable of being taken through the cell's lipid bilayer membrane to an internal compartment (i.e. “internalized”) upon binding to the cell. For example, an internalizing anti-GRP78 antibody is one that is capable of being taken into the cell after binding to csGRP78 on the cell membrane.

In some embodiments, the binding molecule, such as a provided antibody or antigen-binding fragment thereof, when administered to a subject (e.g. alone or as a conjugate) results in a treatment that does not induce an immune response by the subject to the therapy, and/or does not induce such a response to a degree that prevents effective treatment of the disease or condition. In some aspects, the degree of immunogenicity and/or graft versus host response is less than that observed with a different but comparable treatment, such as binding molecule containing the reference antibody. For example, in the case of an antibody drug conjugate (ADC) including the provided anti-GRP78 antibodies, the degree of immunogenicity is reduced compared to a similar ADC including a different antibody that binds to a similar, e.g., overlapping epitope and/or that competes for binding to GRP78 with the provided antibody, such as a mouse antibody. For instance, in provided embodiments, the degree of immunogenicity of an ADC containing a provided anti-GRP78 antibody is reduced compared to similar ADC but that contains a reference antibody.

In some embodiments, a provided binding molecule, such as an antibody or antigen-binding fragment, exhibits features that favor stability of the antibody or binding fragment thereof. In some embodiments, the stability of a provided binding molecule, e.g. antibody or antigen-binding fragment, is evidenced by its thermal stability. Detection of thermal stability of a protein can be used to determine the denaturation effectively and thus is used as a stability test for protein during the development of therapeutic proteins. A skilled artisan is familiar with assays to assess thermal stability of a protein. In some embodiments, a provided binding molecule, such as an antibody or antigen-binding fragment thereof, has high thermal stability and therefore is less prone to protein aggregation so that activity and/or purity of the protein is improved.

In some embodiments, the binding molecules, such as antibodies or antigen-binding fragments thereof, have an onset temperature of aggregation (Tagg) that is greater than about 67° C., greater than about 68° C., greater than about 69° C., greater than about 70° C., greater than about 71° C., or greater than about 72° C., or is any value between any of the foregoing. In some embodiments, the Tagg is between about 67° C. and about 71° C. In some embodiments, the Tagg of the antibody or antigen-binding fragment is about 67° C., about 68° C., about 69° C., about 70° C., or about 71° C.

In some embodiments, the binding molecules, such as antibodies or antigen-binding fragments thereof, have a Tagg that is increased or improved compared to a reference antibody, such as an antibody containing the V_{H} and V_{L} set forth in SEQ ID NOS: 1 and 3, respectively. In some embodiments, the Tagg is increased greater than about 1° C., greater than about 2° C., greater than about 3° C., greater than about 4° C. or greater than about 5° C. compared to a reference antibody (such as an antibody containing the VH and V_{L} set forth in SEQ ID NOS: 1 and 3, respectively).

In some embodiments, the binding molecules, such as antibodies or antigen-binding fragments thereof, have a melting temperature (Tm) for unfolding that is greater than 60° C. In some embodiments, the Tm is greater than about 61° C., greater than about 62° C., greater than about 63° C., greater than about 64° C., greater than about 65° C., greater than about 66° C., greater than about 67° C., greater than about 68° C., greater than about 69° C. or greater than about 70° C., or any value between any of the foregoing. In some embodiments, the Tm is between about 60° C. and about 70° C. In some embodiments, the Tm is about 64° C., about 65° C., about 66° C., about 67° C., about 68° C., or about 69° C.

Among provided binding molecules, such as antibodies or antigen-binding fragments, are those that exhibit improved features related to denaturation. Denaturation is the transition of a protein from the native conformation to an unfolded state and is generally accompanied by a major loss of protein function. As is understood by a skilled artisan, as IgG can be described as a multi-domain protein, the melting curve sometimes shows two transitions, or three transitions, with a first denaturation temperature, Tm1, and a second denaturation temperature Tm2, and optionally a third denaturation temperature Tm3. See e.g. Akazawa-Ogawa et al. Biophys. Rev. 2018, 10:255-258.

In some embodiments, the Tm (e.g. Tm1) is increased or improved compared to a reference antibody, such as an antibody containing the V_{H} and V_{L} set forth in SEQ ID NOS: 1 and 3, respectively. In some embodiments, the Tm (e.g. Tm1) is increased greater than about 2° C., greater than about 3° C., greater than about 4° C., greater than about 5° C., greater than about 6° C., greater than about 7° C., greater than about 8° C., greater than about 9° C., or greater than about 10° C. compared to a reference antibody (e.g. an antibody containing the V_{H} and V_{L} set forth in SEQ ID NOS: 1 and 3, respectively). In some embodiments, the Tm (e.g. Tm1) is increased greater than about 5° C. compared to a reference antibody (e.g. an antibody containing the V_{H} and V_{L} set forth in SEQ ID NOS: 1 and 3, respectively). In some embodiments, the Tm (e.g. Tm1) is increased greater than about 10° C. compared to a reference antibody (e.g. an antibody containing the V_{H} and V_{L} set forth in SEQ ID NOS: 1 and 3, respectively).

In some embodiments, the binding molecules, such as antibodies or antigen-binding fragments thereof, have a Tm1 for unfolding that is greater than 60° C. In some embodiments, the Tm1 is greater than about 61° C., greater than about 62° C., greater than about 63° C., greater than about 64° C., greater than about 65° C., greater than about 66° C., greater than about 67° C., greater than about 68° C., greater than about 69° C. or greater than about 70° C., or any value between any of the foregoing. In some embodiments, the Tm1 is between about 60° C. and about 70° C. In some embodiments, the Tm1 is about 64° C., about 65° C., about 66° C., about 67° C., about 68° C., or about 69° C.

In some embodiments, among provided binding molecules, such as antibodies or antigen-binding fragments, are those in which the denaturation curve for unfolding is monophasic such that only a single Tm, i.e. TM1, is observed. In some embodiments, without wishing to be bound by theory, it is believed that this property of such antibodies may reduce the likelihood of aggregation and hence improve purity and/or activity of such an antibody preparation. For instance, typically due to the different domains of an antibody folded and unfolded domains may be present at different denaturation temperatures, which can lead to reduced heat resistance and thereby increase tendency to aggregate or have reduced activity. An antibody preparation with a single transition (e.g. monophasic thermal denaturation) would be expected to exhibit improved thermostability and developability.

### Nucleic Acids and Expression Vectors

The present disclosure also provides a nucleic acid, e.g. polynucleotide, encoding any one of the provided binding molecules, including any one of the provided anti-GRP78 antibody or antigen-binding fragment thereof, or a chain thereof. The provided nucleic acid of the present disclosure may comprise a polynucleotide sequence encoding a heavy and/or light chain of any one of the antibodies or antigen-binding fragments thereof disclosed herein. In some embodiments, the nucleic acid is an isolated nucleic acid. In some embodiments, the nucleic acid is a recombinant or synthetic nucleic acid. In some embodiments, the nucleic acid is a complementary DNA (cDNA).

In some embodiments, the nucleic acid encodes a heavy chain of an antibody or antigen-binding fragment provided herein, such as any described above. In some embodiments, the nucleic acid encodes a light chain of an antibody or antigen-binding fragment provided herein, such as any described above. In some embodiments, the nucleic acid encodes a heavy chain and a light chain of an antibody or antigen-binding fragment provided herein, such as any described above. The nucleotide sequence of such nucleic acids can be deduced by those skilled in the art by any suitable technique, including but not limited to using the information provided by Table 2, Table 3 and Table 4 herein.

In some embodiments, the heavy chain variable region is encoded by a nucleic acid comprising a polynucleotide sequence having has at least 80% sequence identity to SEQ ID NO:1. In some embodiments, the heavy chain variable region is encoded by a nucleic acid comprising a polynucleotide sequence having has at least 85% sequence identity to SEQ ID NO:1. For instance, the heavy chain variable region is encoded by a nucleic acid comprising a polynucleotide sequence having has at least 86%, 87%, 87%, 88%, 89%, 90%, 91%, 92%, 93% 94%, or 95% sequence identity to SEQ ID NO:1. In some embodiments, the light chain variable region is encoded by a nucleic acid comprising a polynucleotide sequence having has at least 80% sequence identity to SEQ ID NO:3. In some embodiments, the light chain variable region is encoded by a nucleic acid comprising a polynucleotide sequence having has at least 85% sequence identity to SEQ ID NO:3. For instance, the light chain variable region is encoded by a nucleic acid comprising a polynucleotide sequence having at least 86%, 87%, 87%, 88%, 89%, 90%, 91%, 92%, 93% 94%, or 95% sequence identity to SEQ ID NO: 3.

In some embodiments, the heavy chain variable region is encoded by a nucleic acid comprising a polynucleotide sequence having has at least 80% sequence identity to SEQ ID NO: 2. In some embodiments, the heavy chain variable region is encoded by a nucleic acid comprising a polynucleotide sequence having has at least 85% sequence identity to SEQ ID NO: 2. For instance, the heavy chain variable region is encoded by a nucleic acid comprising a polynucleotide sequence having has at least 86%, 87%, 87%, 88%, 89%, 90%, 91%, 92%, 93% 94%, or 95% sequence identity to SEQ ID NO: 2. In some embodiments, the light chain variable region is encoded by a nucleic acid comprising a polynucleotide sequence having has at least 80% sequence identity to SEQ ID NO: 4. In some embodiments, the light chain variable region is encoded by a nucleic acid comprising a polynucleotide sequence having has at least 85% sequence identity to SEQ ID NO: 4. For instance, the light chain variable region is encoded by a nucleic acid comprising a polynucleotide sequence having at least 86%, 87%, 87%, 88%, 89%, 90%, 91%, 92%, 93% 94%, or 95% sequence identity to SEQ ID NO: 4.

Another aspect of this disclosure provides a vector comprising any one of the nucleic acids disclosed herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector.

Also provided is a host cell comprising any of the vectors or nucleic acids disclosed herein. The host cell may be of eukaryotic, prokaryotic, mammalian, or bacterial origin. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains (e.g., V_{H} region and/or V_{L} region) may be expressed in yeast. See, e.g., U.S. Publication No. US 2006/0270045 A1. In some embodiments, a particular eukaryotic host cell is selected based on its ability to make desired post-translational modifications to the heavy chains and/or light chains (e.g., VH region and/or V_{L} region). For example, in some embodiments, CHO cells produce polypeptides that have a higher level of sialylation than the same polypeptide produced in 293 cells.

A method of producing an antibody or antigen-binding fragment thereof that binds to GRP78 is also provided herein, wherein the method comprises culturing the host cell. For recombinant production of the anti-GRP78 antibody, a nucleic acid sequence(s) encoding an antibody, e.g., as described above, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the anti-GRP78 antibody is provided, wherein the method comprises culturing a host cell comprising a nucleic acid sequence encoding the antibody, as provided above, under conditions suitable for expression of the antibody, and optionally recovering or purifying the antibody from the host cell (or host cell culture medium).

In some embodiments, a nucleic acid of the present disclosure may be operably linked to a transcriptional control element, e.g., a promoter, and enhancer, etc. Suitable promoter and enhancer elements are known to those of skill in the art.

In certain embodiments, the nucleic acid is in operable linkage with a promoter. For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; phosphoglycerate kinase-1 (PGK) promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, this disclosure should not be limited to the use of constitutive promoters. Inducible promoters are also contemplated as part of this disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in any way as limiting: Bacterial: pBs, phagescript, PsiX174, pBluescript SK, pBs KS, pNH8a, pNH16a, pNH18a, pNH46a; pTrc99A, pKK223-3, pKK233-3, pDR540, and pRIT5. Eukaryotic: pWLneo, pSV2cat, pOG44, PXR1, pSG, pSVK3, pBPV, pMSG and pSVL.

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the nucleic acid into a host cell. Accordingly, an expression vector (e.g., a lentiviral vector) of the present disclosure may comprise a nucleic acid encoding a polypeptide. In some embodiments, the expression vector (e.g., lentiviral vector) will comprise additional elements that will aid in the functional expression of the polypeptide encoded therein. In some embodiments, an expression vector comprising a nucleic acid encoding for a polypeptide further comprises a mammalian promoter. In some embodiments, the vector further comprises an elongation-factor-i-alpha promoter (EF-1a promoter). Use of an EF-1a promoter may increase the efficiency in expression of downstream transgenes. Physiologic promoters (e.g., an EF-1a promoter) may be less likely to induce integration mediated genotoxicity and may abrogate the ability of the retroviral vector to transform stem cells. Other physiological promoters suitable for use in a vector (e.g., lentiviral vector) are known to those of skill in the art and may be incorporated into a vector of the present disclosure. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of this disclosure. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory present element (WPRE). Accordingly, in some embodiments a vector for the present disclosure further comprises a WPRE sequence. Various posttranscriptional regulator elements are known to those of skill in the art and may be incorporated into a vector (e.g., lentiviral vector) of the present disclosure. A vector of the present disclosure may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and viral replication. In some embodiments, a vector (e.g., lentiviral vector) of the present disclosure includes a 3′ U3 deleted LTR. Accordingly, a vector (e.g., lentiviral vector) of the present disclosure may comprise any combination of the elements described herein to enhance the efficiency of functional expression of transgenes. For example, a vector (e.g., lentiviral vector) of the present disclosure may comprise a WPRE sequence, cPPT sequence, RRE sequence, 5′LTR, 3′ U3 deleted LTR′ in addition to a nucleic acid encoding for a CAR.

Vectors of the present disclosure may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed on further. Accordingly, self-inactivating vectors may greatly reduce the risk of creating a replication-competent virus.

In some embodiments, a nucleic acid of the present disclosure may be RNA, e.g., in vitro synthesized RNA. Methods for in vitro synthesis of RNA are known to those of skill in the art; any known method can be used to synthesize RNA comprising a sequence encoding a polypeptide of the present disclosure. Methods for introducing RNA into a host cell are known in the art. See, e.g., Zhao et al. Cancer Res. (2010) 15: 9053. Introducing RNA comprising a nucleotide sequence encoding a polypeptide of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

In order to assess the expression of a polypeptide (e.g. an antibody) or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present disclosure is provided for the production of an antibody or antigen-binding fragment as described herein, e.g., in a host cell. In some embodiments, a nucleic acid of the present disclosure provides for amplification of the polypeptide-encoding nucleic acid.

### Antibody Drug Conjugates (ADCs)

Also provided in this disclosure are immunoconjugates, such as antibody drug conjugates (ADCs), comprising any of the antibodies or antigen-binding fragments thereof disclosed herein linked or conjugated to a drug (e.g. a cytotoxic drug), or a toxin, or a radioisotope molecule. In particular embodiments, the ADC is composed of an antibody or antigen-binding fragment disclosed herein and a cytotoxic drug, such as MMAE or SN38. In certain embodiments, the ADCs of this disclosure can be specifically targeted to GRP78 expressing cells in order to effectively eradicate said cells. In provided embodiments, the ADC is able to be internalized by the cell upon binding of the antibody or antigen-binding fragment to GRP78, such as human GRP78.

Exemplary antibodies or antigen-binding fragments in a provided ADC include any as described herein, such as in section “Antibodies and Antigen-Binding Fragments.”

In some embodiments, the immunoconjugate, such as an ADC, has the formula Ab-(L-D)x, wherein Ab is the antibody or antigen-binding fragment thereof, such as any described herein; L is a linker; and D is a cytotoxic drug and x indicates the number of copies of (L-D) bonded to Ab and is from 1 to 8.

In certain embodiments, the antibody portion of the ADC comprises an antibody or antigen-binding fragment thereof comprising one of more CDR, variable light chain, variable heavy chain, and/or constant region of antibody B4 (preferably SEQ ID NOs. 1, 3, 5-10, 17, 18, 19, 21, and/or 23, and/or one or derivative(s) thereof) or antibody F6 (preferably SEQ ID NOs. 2, 4, 11-16, 17, 18, 20, 22, and/or 24, and/or one or derivative(s) thereof) (see the preferred amino acid sequences shown in Table 3). In preferred embodiments, a B4 antibody-based ADC includes SEQ ID NOs. 5-10, and/or one or more derivatives thereof. In preferred embodiments, a B4 antibody-based ADC includes SEQ ID NOs. 1 and 3; SEQ ID NOs. 17 and/or 18; or SEQ ID NOs. 21 and/or 23; or SEQ ID NO: 19; and/or one or more derivatives thereof. In preferred embodiments, an F6 antibody-based ADC includes SEQ ID NOs. 11-16, and/or one or more derivatives thereof. In preferred embodiments, an F6 antibody-based ADC includes SEQ ID NOs. 2 and 4; SEQ ID NOs. 17 and/or 18; or SEQ ID NOs. 22 and/or 24; or SEQ ID NO: 20; and/or one or more derivatives thereof.

Any of a variety of cytotoxic drugs may be employed as known to a skilled artisan. The antibodies or antigen-binding fragments thereof of the current disclosure can be linked or conjugated to any drug or cytotoxic agent known to one of skill in the art, including but not limited to maytansinoid (DM1), or SSTR2-binding octreotide, or toxin, including but not limited to paclitaxel, dauxrubicin, duocarmycin A, 5-fluoruracil, methotrexate, tubulin polymerization inhibitors, ravtansine (DM4), and Ricin A. In certain embodiments, the ADCs of this disclosure may be linked or conjugated to an auristatin including but not limited to monomethyl auristatin E (MMAE) or monomethyl auristatin F (MMAF). In certain embodiments, the ADCs of this disclosure may be linked or conjugated to an active metabolite of a drug. A nonlimiting example of such drug is SN-38, an active metabolite of irinotecan. One of skill in the art would be able to select a drug or cytotoxic agent to conjugate to the antibodies of this disclosure based on the desired properties of the drug or cytotoxic agent and the properties of the cell to be targeted by the ADC. Exemplary cytotoxic drugs suitable for use in the present disclosure are further described below. In some embodiments, the cytotoxic drug is monomethyl auristatin E (MMAE). In some embodiments, the cytotoxic drug is a camptothecin analog. In some embodiments, the cytotoxic drug is Exatecan. In some embodiments, the cytotoxic drug is DXd (the hydroxyacetyl amide of Exatecan)

Any of a variety of linkers may be employed as known to a skilled artisan. Exemplary linkers are described below. In some embodiments, the linker is a cleavable linker. In some embodiments, the linker is a cathepsin-cleavable linker. In some embodiments, the linker is a pH cleavable linker. In some embodiments, the linker is a non-cleavable linker.

In some embodiments, the drug antibody ratio (DAR) in a provided conjugate is from about 4 to about 8. In some embodiments, the DAR is about 4. In some embodiments, the DAR is about 8.

In certain embodiments, among the provided ADCs are those in which the antibody or antigen-binding fragment is internalized into a target cell upon binding, e.g., into a degradative compartment in the cell. In some embodiments, provided ADCs are thus those that internalize upon binding to a target cell, undergo degradation, and release the drug moiety to kill cancer cells. The drug moiety may be released from the antibody and/or the linker moiety of the ADC by enzymatic action, hydrolysis, oxidation, or any other mechanism. For instance, in some embodiments, the antibody of the ADC binds to GRP78 expressed on the surface of a cell (e.g. cancer cell) and enters the cell upon binding. In some embodiments, the drug moiety of the ADC is released from the antibody moiety of the ADC after the ADC enters and is present in a cell expressing the csGRP78 antigen (i.e., after the ADC has been internalized).

Cytotoxic Drugs Various cytotoxic drugs can be used in the various embodiments disclosed herein. Although the recitation below is not exhaustive, the following cytotoxic drugs (payloads) are of particular interest.

In certain embodiments, the cytotoxic drug is ((S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide, (monomethyl auristatin E, abbreviated as MMAE)), having the structure:

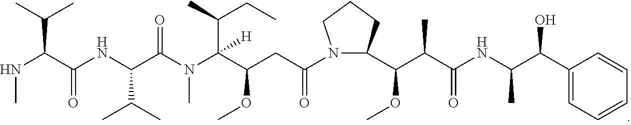 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In certain embodiments, the cytotoxic drug is ((S)-4,11-diethyl-4,9-dihydroxy-1,12-dihydro-14H-pyrano[3′,4′:6,7]indolizino[1,2-b]quinoline-3,14(4H)-dione, (abbreviated as SN38)) having the structure:

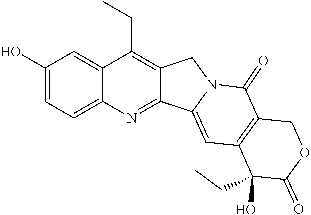 [SMILES: CCc1c2c(nc3ccc(O)cc13)-c1cc3c(c(=O)n1C2)COC(=O)[C@]3(O)CC]

In certain embodiments, the cytotoxic drug is a hexacyclic camptothecin analog.

In certain embodiments, the cytotoxic drug is the hexacyclic camptothecin analog, Exatecan, (1S,9S)-1-amino-9-ethyl-5-fluoro-9-hydroxy-4-methyl-1,2,3,9,12,15-hexahydro-10H, 13H-benzo[de]pyrano[3,4′:6,7]indolizino[1,2-b]quinoline-10,13-dione, having the structure:

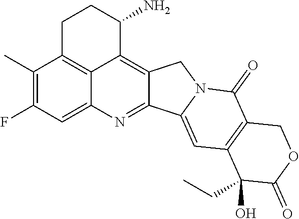 [SMILES: CC[C@@]1(O)C(=O)OCc2c1cc1n(c2=O)Cc2c-1nc1cc(F)c(C)c3c1c2[C@@H](N)CC3]

In certain embodiments, the cytotoxic drug is DXd (or DX-8951), N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide (the 2-hydroxyacetyl amide of Exatecan)), having the structure:

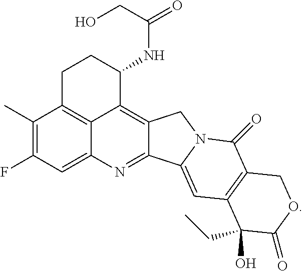 [SMILES: CC[C@@]1(O)C(=O)OCc2c1cc1n(c2=O)Cc2c-1nc1cc(F)c(C)c3c1c2[C@@H](NC(=O)CO)CC3]

In certain embodiments, the cytotoxic drug is selected from any cytotoxic drug, including but not limited to maytansinoids, auristatins including MMAE, MMAF and mertansine, taxoids, DNA binders including calicheamicins, topoisomerase inhibitors including SN-38, Exatecan and DXd, camptothecins, CC1065 analogs, duocarmycins, amatoxins, dexamethone, and budesonide and other cytotoxic agents mentioned herein and/or not mentioned herein.

In certain embodiments, the cytotoxic drug is selected from the group consisting of compounds selected from the generic structure:

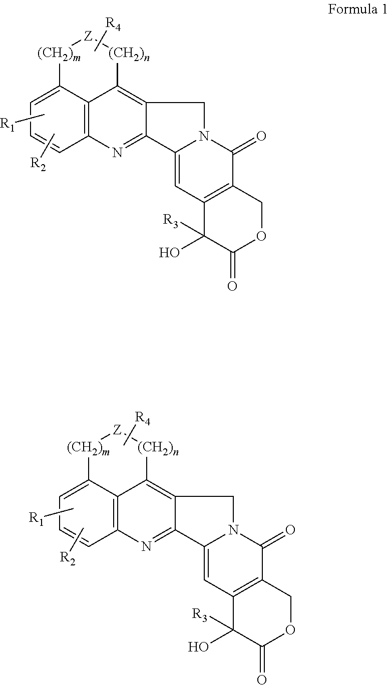

wherein R₁ and R₂ individually represent a hydrogen atom, a hydroxyl group, a C₁₋₆alkyl group (“C₁₋₆ alkyl group” means an alkyl group having 1 to 6 carbon atoms, herein-after defined in the same manner) which may contain a halogen atom, a nitro or cyano group, a C₁₋₆ alkenyl group, a C₁₋₆ alkynyl group, a C₁₋₆ alkoxyl group, a C₁₋₆ aminoalkoxyl group, a halogen atom, a nitro group, a cyano group, a mercapto group, an alkylthio group, an amino group which may contain a protective group, a C₁₋₆ aminoalkyl group which may contain a protective group or a C₁₋₆ alkyl group at the amino-position, a C1-6 aminoalkylamino group which may contain a protective group or a C₁₋₆ alkyl group at the amino-position, a C₁₋₆ alkyl group with a heterocyclic ring which may contain a C₁₋₆ alkyl, alkoxyl, amino, halogen, nitro, or cyano group, a carbonyl with a heterocyclic ring which may contain a C₁₋₆ alkyl, C₁₋₆ alkoxyl, amino, halogen, nitro, or cyano group, a C₁₋₆ alkylamino group with a heterocyclic ring which may contain C₁₋₆ alkyl, C₁₋₆ alkoxyl, amino (which may contain a protective group), halogen, nitro, cyano or a protective group, an amino-heterocyclic group which may contain a protective group or a C₁₋₆ alkyl group at the nitrogen atom of the heterocyclic ring moiety or at the amino position, or a carbamoyl group which may contain a protective group or a C₁₋₆ alkyl group; R₃ represents a C₁₋₆ alkyl group; R₄ represents an amino group which may contain a protective group, a quaternary trialkyl ammonium such as —N+(CH₃)₃, a C₁₋₆ alkylamino group which may contain a protective group, a C₁₋₆ aminoalkyl group which may contain a protective group, a C₁₋₆ alkylaminoalkyl group which may contain a protective group, a sulfonic acid group, or a carboxyl group; Z represents an oxygen atom, a sulfur atom, CR₅R₆, wherein R₅ and R₆ individually represent a hydrogen atom or a C₁₋₆ alkyl, or N—R₇, wherein R₇ stands for a hydrogen atom, a C₁₋₆ alkyl group, a C1.6 aminoalkyl group which may contain a protective group, a C₁₋₆ alkylaminoalkyl group which may contain a protective group, or a protective group for the amino group; and m and n individually represent 0, 1 or 2. Such compounds are disclosed in U.S. Pat. No. 5,834,476, which is incorporated into this application in its entirety.

Among the compounds of Formula 1, those in which the asymmetric carbon at position 10 (in IUPAC nomenclature) bearing the hydroxyl group take the S-type configuration are preferred for increased medicinal activity. See also, for example, the (S) configuration of DXd and Exatecan.

### Linkers

In certain embodiments, the antibody or antigen-binding fragment of the present disclosure (Ab) is conjugated to a cytotoxic drug (D) via a linker (L). In certain embodiments, the linker comprises an antibody conjugating terminus and a carboxy terminus. In certain embodiments, the antibody conjugating terminus comprises a thiol-conjugating terminus. In certain embodiments, the antibody conjugating terminus comprises a disulfide-conjugating terminus. In certain embodiments, thiol-conjugating terminus comprises a Michael acceptor. In certain embodiments, the thiol-conjugating terminus is conjugated to the antibody via a Michael addition between a Michael acceptor of the linker and a cysteine of the antibody. In certain embodiments, the Michael acceptor is a maleimide. In certain embodiments, the disulfide-conjugating terminus comprises a moiety selected from the group consisting of a bis-sulfone, 3,4-disubstituted maleimide, bromomaleimide, and/or allyl sulfone. Further examples of disulfide-conjugating moieties are found in Kuan et al. (Chem. Eur. J. 2016, 22(48):17112-17129) and US20220062436A1 from Abzena UK Ltd.

In certain embodiments, the bis-sulfone comprises a 3-(arylsulfonyl)-2-((arylsulfonyl)methyl)propanoyl moiety, wherein aryl is phenyl or p-tolyl. In certain embodiments, the bis-sulfone comprises a 3-(pegylated sulfonyl)-2-((pegylated sulfonyl)methyl)propanoyl moiety, wherein the PEG group may be -PEG₇-Me. In certain embodiments, the disulfide-conjugating terminus is conjugated to the antibody via a reaction with the the 3-(arylsulfonyl)-2-((arylsulfonyl)methyl)propanoyl moiety of the linker via a reduced disulfide of the antibody via consecutive Michael and retro-Michael reactions with the two sulfides produced through the reduction to produce add the L-D by rebridging the disulfide of the antibody.

In certain embodiments, the linker is conjugated to the cytotoxic drug via a carbamate. In certain embodiments, the carbamate is derived from an amine of the cytotoxic drug and a reactive carbonate formed on the linker. In certain embodiments, the linker is conjugated to the cytotoxic drug via a carbonate ester. In certain embodiments, the carbonate ester is derived from an alcohol moiety of the cytotoxic drug and a carboxylic acid group of the linker. In certain embodiments, the linker is conjugated to the cytotoxic drug via an amide. In certain embodiments, the amide is derived from reaction of an amine of the cytotoxic drug and a carboxyl group of the linker. In certain embodiments, the linker is branched and conjugated to more than one cytotoxic drug.

The linkers may comprise ethylene glycol/propylene glycol moieties. For example, in some embodiments both the first linking component and the second linking component (and thus the overall linker) contain ethylene glycol/propylene glycol repeats. Ethylene glycol/propylene glycol-based linkers may provide distinct advantages over linkers not containing ethylene glycol/propylene glycol. For example, ethylene glycol/propylene glycol-based linkers may provide greater solubility or less propensity for aggregation of the ADC in which they are incorporated. For further example, ethylene glycol/propylene glycol-based linkers may provide greater flexibility, so that, for example, in an instance where the linker is conjugated to an amino acid residue that is in close proximity to the antigen-binding domain (e.g. the hypervariable region), there would be a reduced chance that the linker/therapeutic agent would bind to or interact with the surface elements of the antigen/receptor, thus making the overall immunoconjugate more therapeutically effective. For still further example, ethylene glycol/propylene glycol-based linkers may provide greater flexibility, and greater solubility and/or less propensity for aggregation. Additionally, in such embodiments where the linker is generated through click chemistry (e.g. using DBCO and organic azides), while not wishing to be bound by theory, the use of ethylene glycol and/or propylene glycol in the linking components may act as a spacer to prevent non-specific interactions (pi-pi interactions) between the tetracyclic ring structure of the triazole moiety generated through click chemistry reactions and any exposed aromatic side chains, especially tryptophan side chains. The ethylene glycol/propylene glycol-based linkers of the present disclosure may comprise repeats of anywhere between 1-30 ethylene glycol and/or propylene glycol units total, e.g. 1-10 ethylene glycol and/or propylene glycol units per each of the first linking component and the second linking component in such embodiments that utilize a first linking component and a second linking component.

The covalent linkages in such immunoconjugates may comprise a cleavable linking moiety, for example a cathepsin cleavable linker such as Val-Cit linker or Val-Cit-PAB, which is cleavable by Cathepsin B inside the lysosome. A commercially available Val-Cit linker has been used as described herein. Other cleavable linking moieties may comprise a Phe-Lys linker, which is also cleavable by Cathespin B. Some of the simplest cleavable linking moieties include disulfide (S-S) bridges, which are cleavable in a reductive (i.e. intracellular) environment. However, cleavable linking moieties such as Val-Cit linkers provide more specificity than, for example, certain disulfide bridges, which may be subject to indiscriminate cleavage, and thus present a superior option, although any such cleavable linking moiety is to be considered within the scope of the present disclosure. An overview of cleavable linking moieties which may be suitable in the context of the present disclosure is provided in Leriche et al. (Bioorg. Med. Chem. 2012, 20(2):571-582), which is hereby incorporated by reference in its entirety.

Alternatively, the linker may be non-cleavable. Non-cleavable linkers may comprise any linking moiety that is resistant to cleavage in an intracellular environment.

In certain embodiments, the linker reagent forms a covalent linkage to connect the antibody and the cytotoxic drug to form the antibody drug conjugate and comprises a maleimide linking moiety MC-Val-Cit-PAB having the structure:

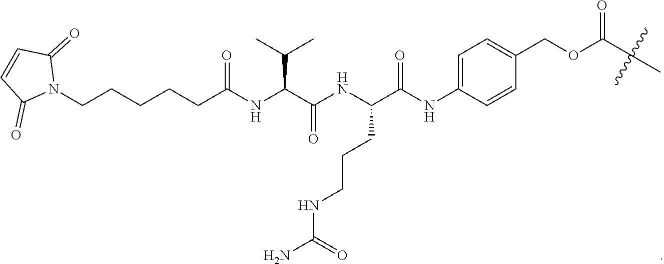 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1]

wherein the incomplete bond represented by the frilled line represents the attachment point of a cytotoxic drug (D).

In certain embodiments, the maleimide linker reagent comprises CL2A, having the structure:

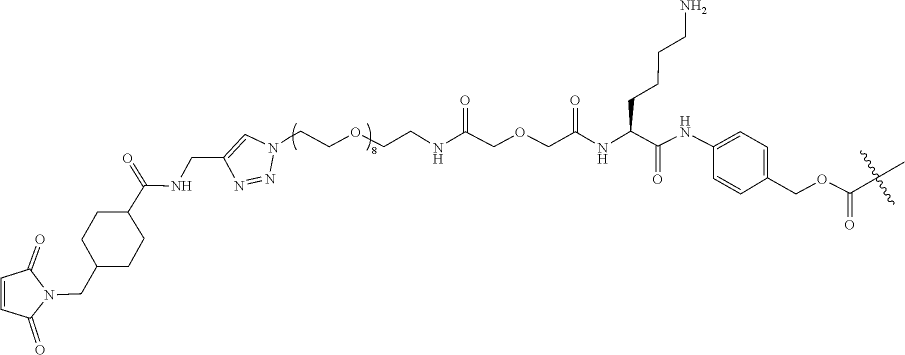

wherein the incomplete bond represented by the frilled line represents the attachment point of a cytotoxic drug (D).

In certain embodiments, the linker reagent that reacts to form the antibody drug conjugate comprises a disulfide crosslinking reagent TBR-Glu-(Val-Cit-PAB)-amino-PEG₂₄-Me having the structure:

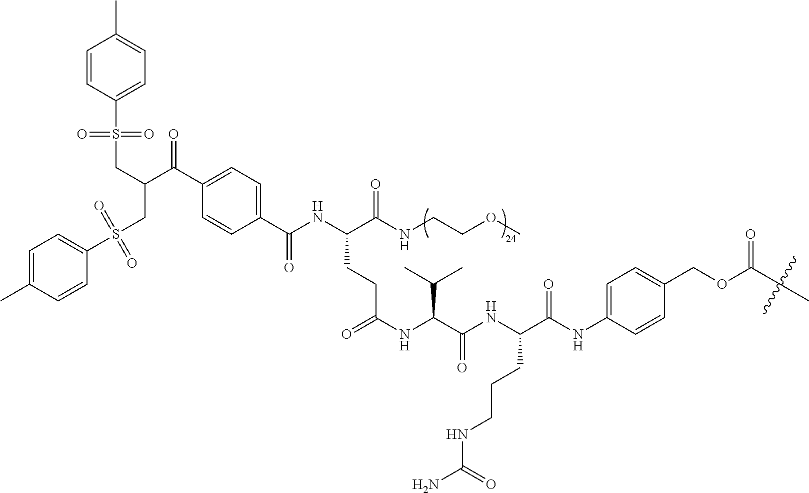

wherein the incomplete bond represented by the frilled line represents the attachment point of a cytotoxic drug (D).

In certain embodiments, the disulfide crosslinking reagent comprises TBR-[Glu-(Val-Cit-PAB-SN38)]₂-Glu-(amino-PEG₂₄-Me)₂ having the structure:

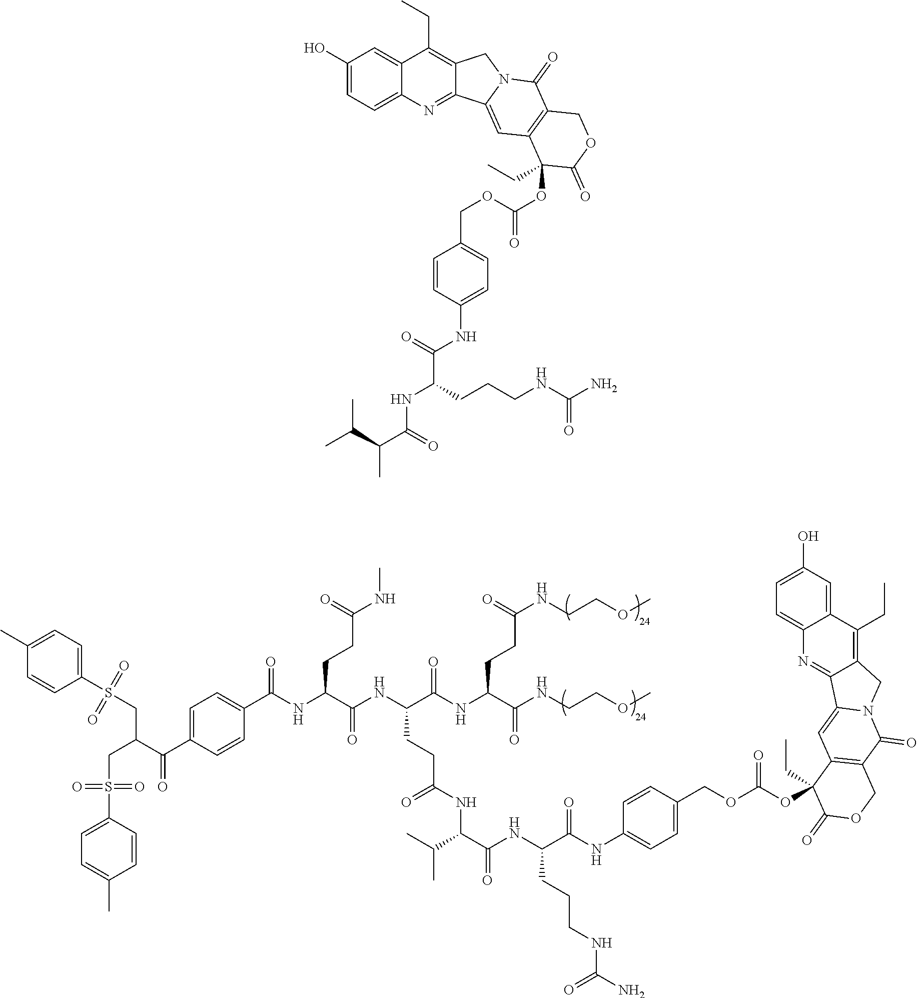

wherein the incomplete bond represented by the frilled line represents the attachment points of two copies of a cytotoxic drug (D).

In certain embodiments, the L-D reagent comprises a reactive moiety with a cleavable linker bonded to a MMAE cytotoxin, MC-Val-Cit-PAB-MMAE, having the structure:

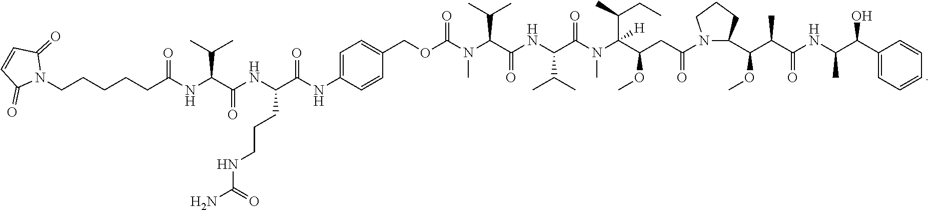 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

In certain embodiments, L-D comprises a reactive moiety with a cleavable linker bonded to a cytotoxin which is a camptothecin analog or derivative such as CL2A-7-ethyl-10-hydroxycamptothecin, or CL2A-SN38, having the structure:

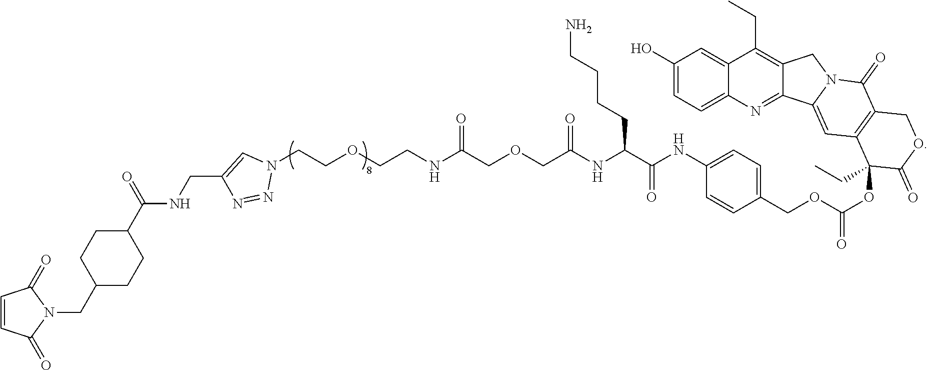

In certain embodiments, L-D reagent comprises a reactive moiety with a cleavable linker bonded to a cytotoxin, TBR-Glu-(Val-Cit-PAB-MMAE)-amino-PEG₂₄-Me, having the structure:

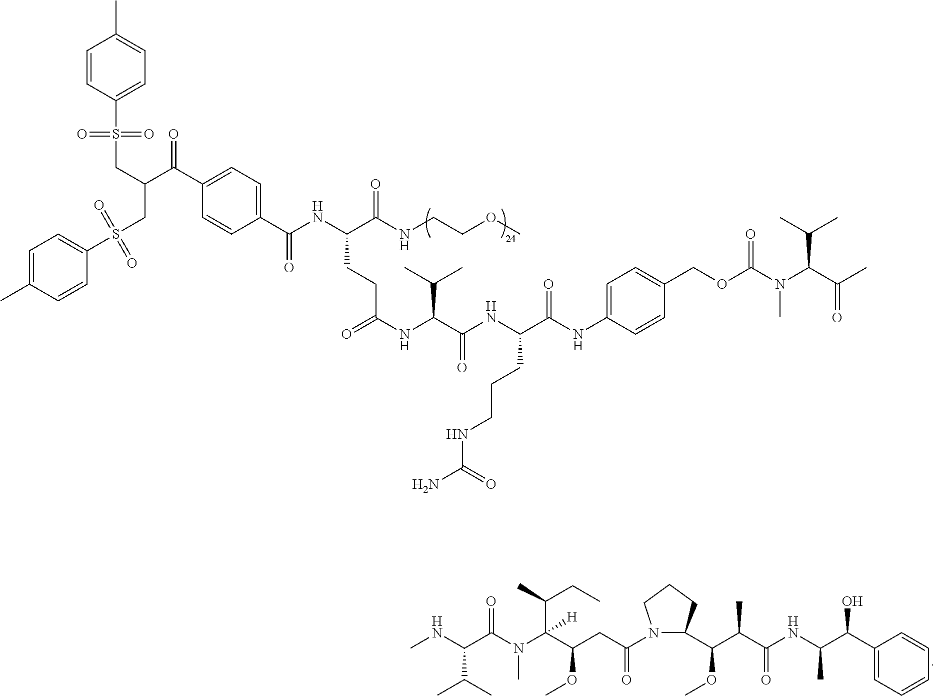

In certain embodiments, the L-D reagent comprises a single cross-linking moiety with two or more cleavable linkers, each bonded to a cytotoxin such as TBR-[Glu(Val-Cit-PAB-SN38)]₂-Glu-(amino-PEG₂₄-Me)₂, having the structure:

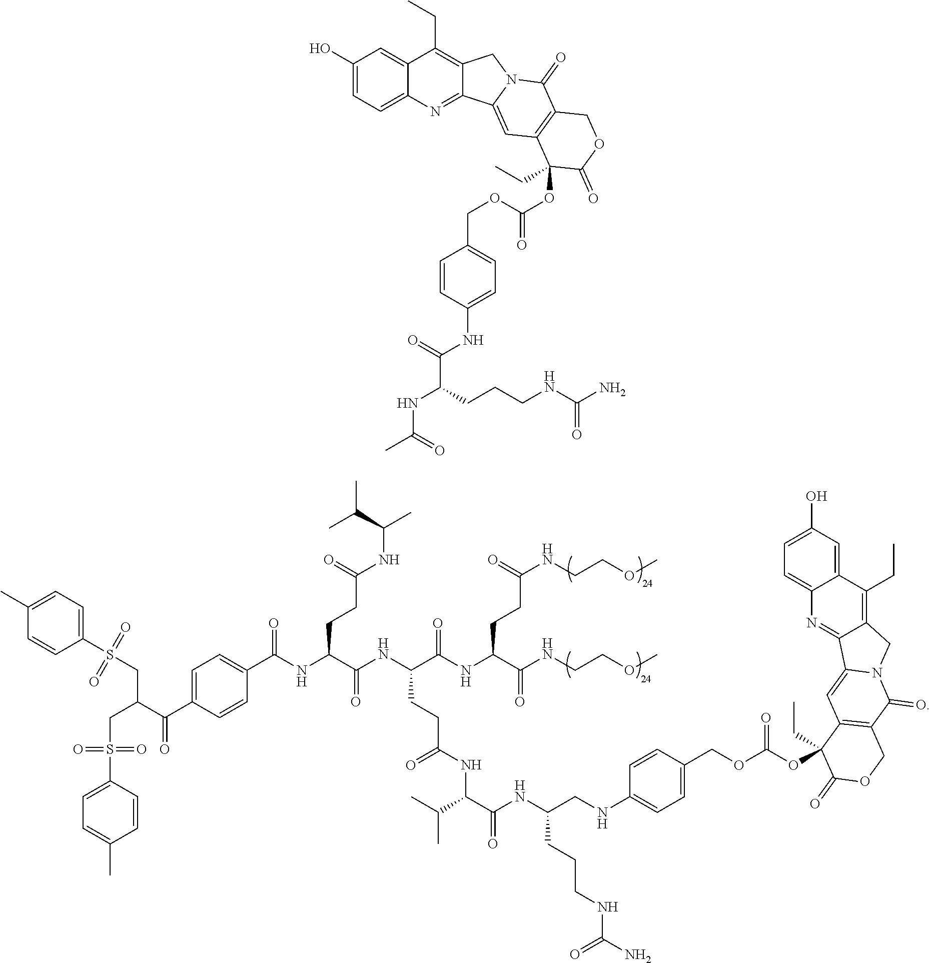

In certain embodiments, the L-D reagent comprises a single cross-linking moiety with two or more cleavable linkers, each bonded to a cytotoxin, such as TBR-Glu-(Val-Cit-PAB-SN38)₂, having the structure:

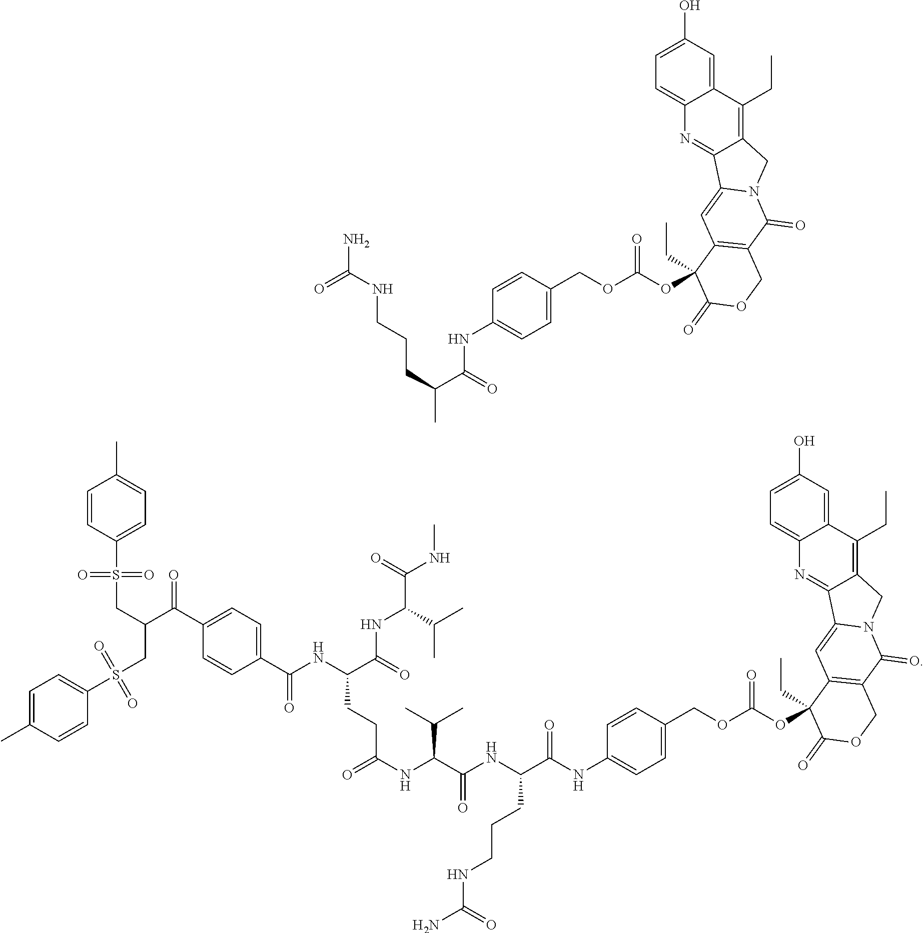 [SMILES: CCc1c2c(nc3ccc(O)cc13)-c1cc3c(c(=O)n1C2)COC(=O)[C@@]3(CC)OC(=O)OCc1ccc(NC(=O)[C@@H](C)CCCNC(N)=O)cc1 ; CCc1c2c(nc3ccc(O)cc13)-c1cc3c(c(=O)n1C2)COC(=O)[C@@]3(CC)OC(=O)OCc1ccc(NC(=O)[C@H](CCCNC(N)=O)NC(=O)[C@@H](NC(=O)CC[C@H](NC(=O)c2ccc(C(=O)C(CS(=O)(=O)c3ccc(C)cc3)CS(=O)(=O)c3ccc(C)cc3)cc2)C(=O)N[C@H](C(=O)NC)C(C)C)C(C)C)cc1]

In certain embodiments, the L-D reagent comprises a reactive moiety with a cleavable linker bonded to a cytotoxin, such as TBLR-GGFG-DXd, having the structure:

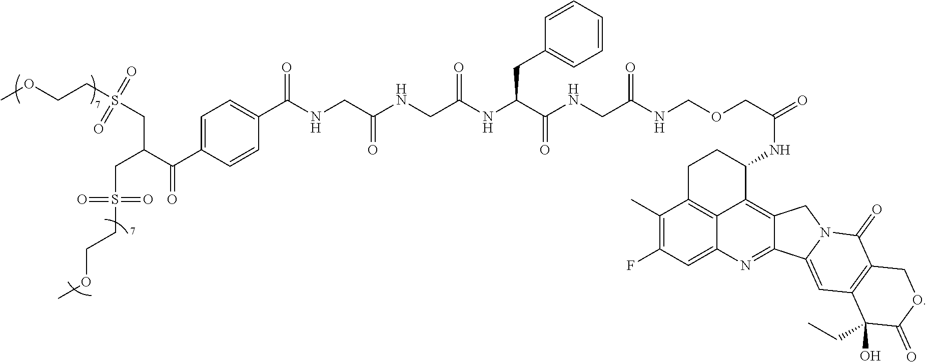

In certain embodiments, the L-D reagent comprises a reactive moiety with two cleavable linkers bonded to two cytotoxins, such as TBLR-Glu-(GGFG-DXd)₂ having the structure:

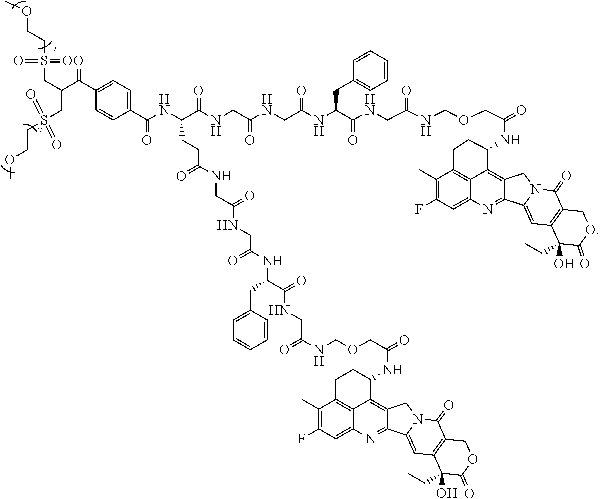

In some embodiments, there is provided an immunoconjugate, such as an ADC, having the formula Ab-(L-D)x, wherein: Ab is an antibody wherein Ab is an antibody that specifically binds to an epitope of GRP78; and the L-D reagent that reacts with the antibody to form the Ab-(L-D)x comprises one of the structures disclosed above and herein.

In some embodiments, there is provided an immunoconjugate, such as an ADC, having the formula Ab-(L-D)x, wherein Ab is an antibody that specifically binds to an epitope of GRP78, wherein the L-D reagent used to make the ADC is TBR-Glu-(Val-Cit-PAB-MMAE)-amino-PEG₂₄-Me, and the Ab-(L-D)x, wherein x is from 1 to 8, has the structure:

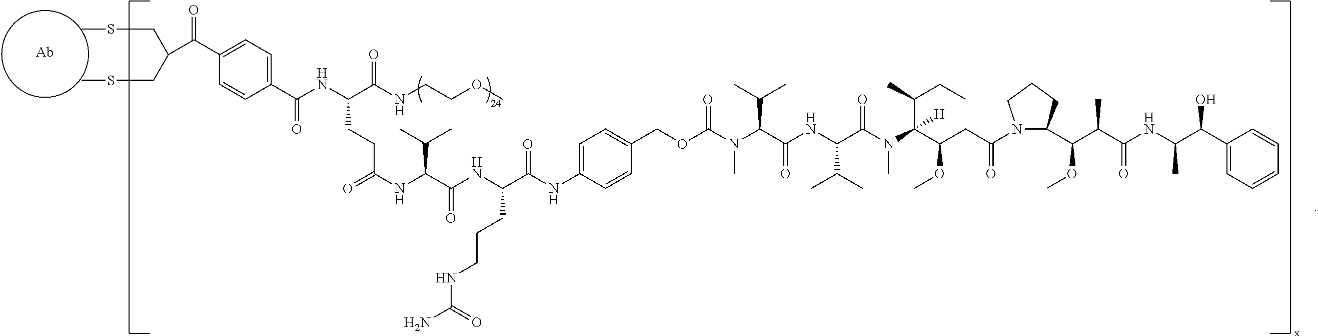

In some embodiments, there is provided an immunoconjugate, such as an ADC, having the formula Ab-(L-D)x, wherein Ab is an antibody that specifically binds to an epitope of GRP78, where the L-D reagent used to make the ADC is TBR-Glu-(Val-Cit-PAB-Exatecan)-amino-PEG₂₄-Me, and the Ab-(L-D)ₓ, wherein x is from 1 to 8, has the structure:

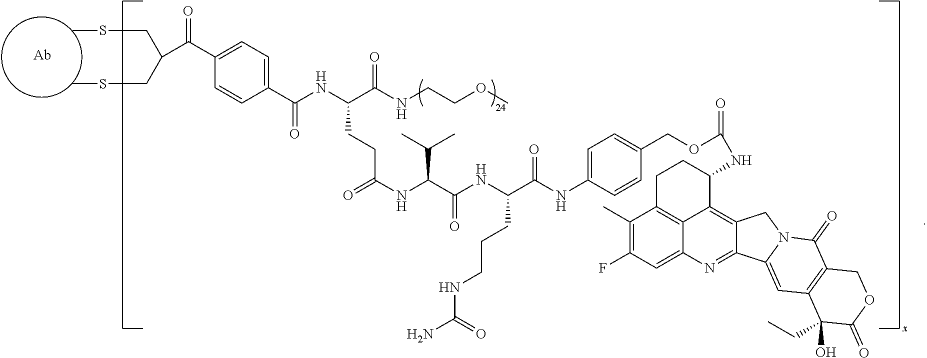

In some embodiments, there is provided an immunoconjugate, such as an ADC, having the formula Ab-(L-D)x, wherein Ab is an antibody wherein Ab is an antibody that specifically binds to an epitope of GRP78, where the L-D reagent used to make the ADC is TBR-Glu-(Val-Cit-PAB-MMAE)-amino-PEG₂₄-Me, and the Ab-(L-D)ₓ, wherein x is from 1 to 8, has the structure:

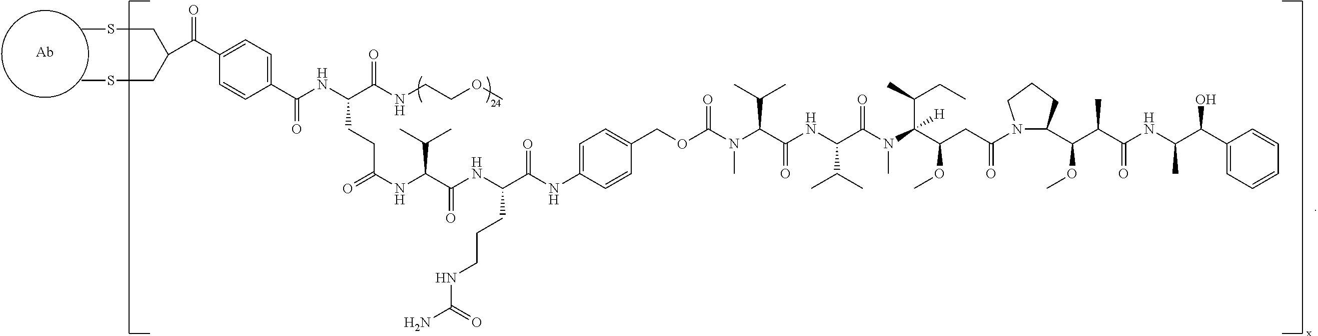

In some embodiments, there is provided an immunoconjugate, such as an ADC, having the formula Ab-(L-D)x, wherein Ab is an antibody that specifically binds to an epitope of GRP78, wherein the L-D reagent used to make the ADC is TBR-[Glu-(GGFG-DXd)]₂-Glu-(amino-PEG₂₄-Me)₂, and the Ab-(L-D)x, wherein x is from 1 to 4, has the structure:

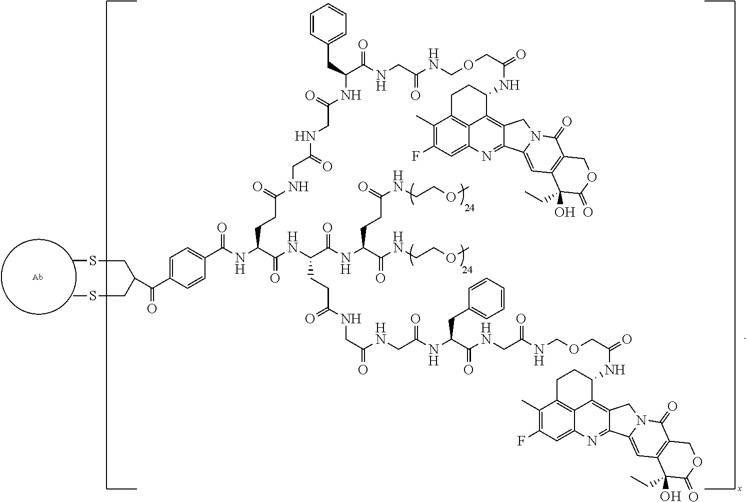

In some embodiments, there is provided an immunoconjugate, such as an ADC, having the formula Ab-(L-D)x, wherein Ab is an antibody wherein Ab is an antibody that specifically binds to an epitope of GRP78, where the L-D regent used to make the ADC is TBLR-GGFG-DXd, and the Ab-(L-D)x, wherein x is from 1 to 8, has the structure:

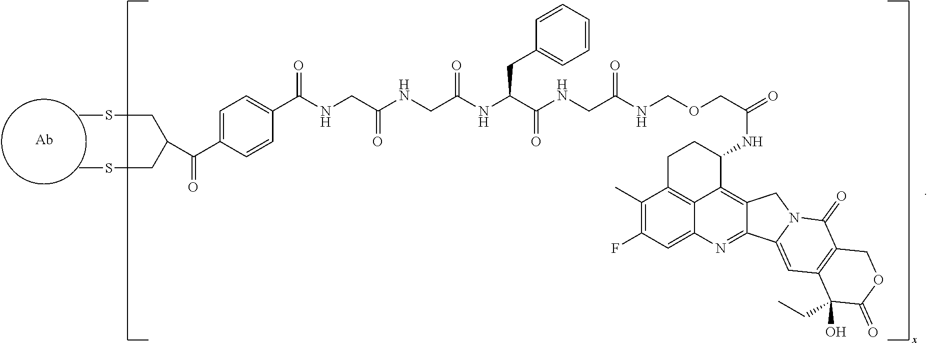

In some embodiments, there is provided an immunoconjugate, such as an ADC, having the formula Ab-(L-D)x, wherein Ab is an antibody that specifically binds to an epitope of GRP78, and the Ab-(L-D)x, wherein the L-D reagent used to make the ADC is TBR-Glu-(Val-Cit-PAB-MMAE)-amino-PEG₂₄-Me, wherein x is from 1 to 8, has the structure:

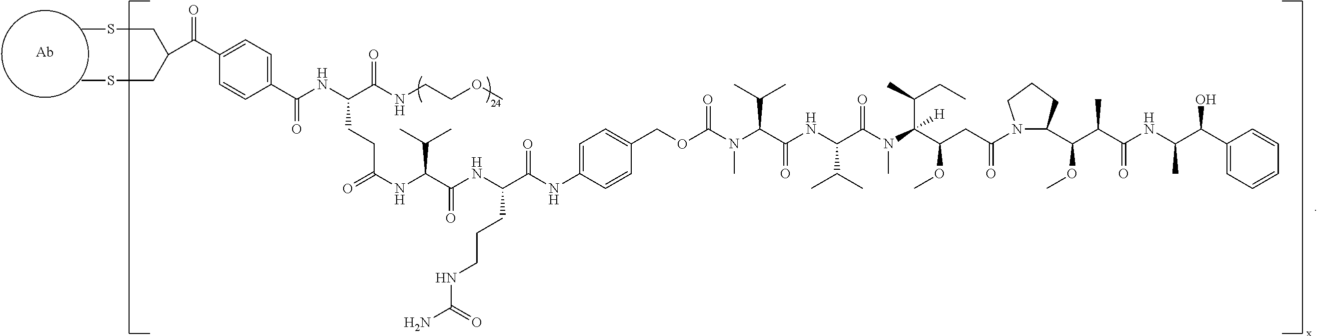

In some embodiments, there is provided an immunoconjugate, such as an ADC, having the formula Ab-(L-D)x, wherein Ab is an antibody that specifically binds to an epitope of GRP78, wherein the L-D reagent used to make the ADC is TBR-Glu-(Val-Cit-PAB-Exatecan)-amino-PEG₂₄-Me and the Ab-(L-D)x, wherein x is from 1 to 8, has the structure:

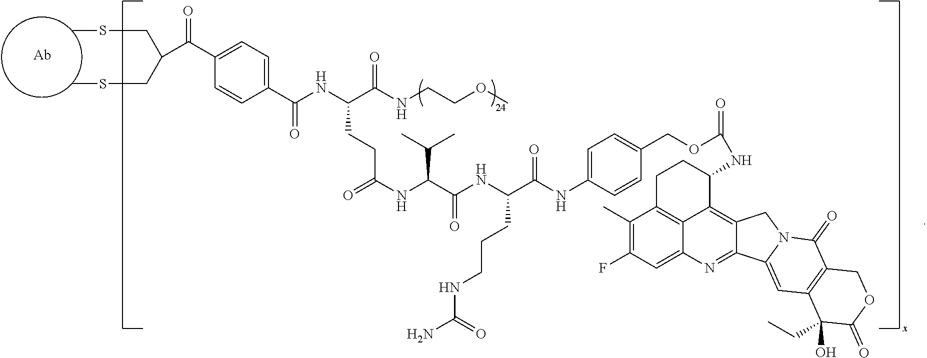

In some embodiments, there is provided an immunoconjugate, such as an ADC, having the formula Ab-(L-D)x, wherein Ab is an antibody wherein Ab is an antibody that specifically binds to an epitope of GRP78, wherein the L-D reagent used to make the ADC is TBR-Glu-(Val-Cit-PAB-MMAE)-amino-PEG₂₄-Me and the Ab-(L-D)x, wherein x is from 1 to 8, has the structure:

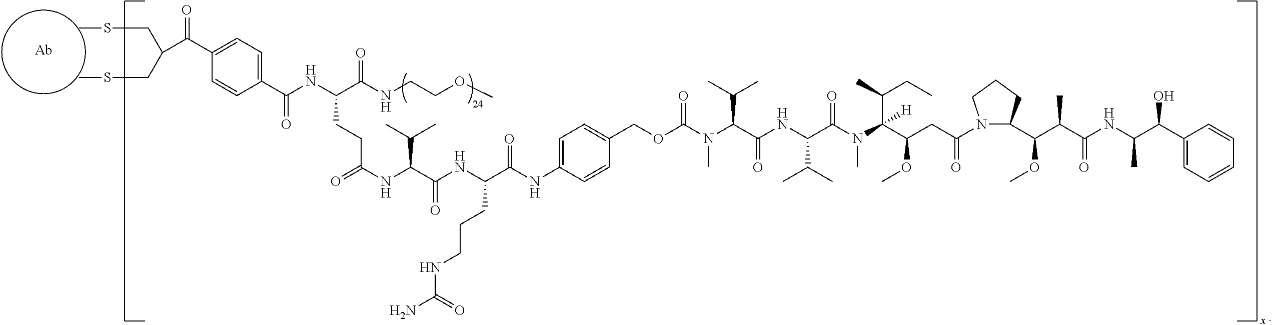

In some aspects of this embodiment, x is 4 and thus the DAR is 4.

In some embodiments, there is provided an immunoconjugate, such as an ADC, having the formula Ab-(L-D)x, wherein Ab is an antibody that specifically binds to an epitope of GRP78, the L-D reagent used to make the ADC is TBR-Val-Cit-PAB-MMAE, and the Ab-(L-D)ₓ, wherein x is from 1 to 8, has the structure:

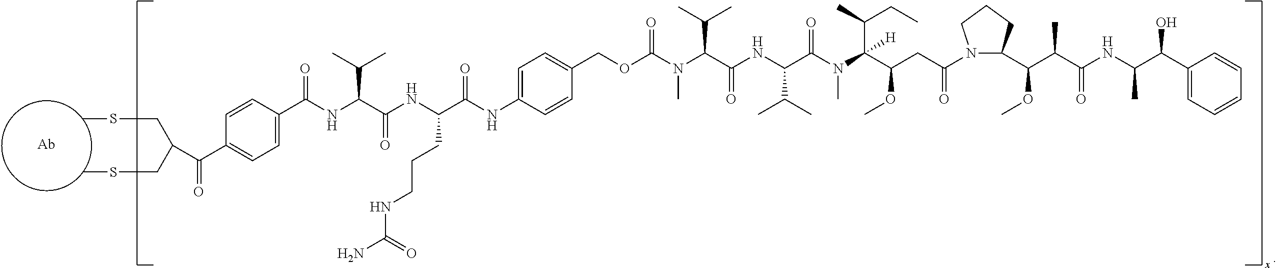

In some embodiments, there is provided an immunoconjugate, such as an ADC, having the formula Ab-(L-D)x, wherein Ab is an antibody wherein Ab is an antibody that specifically binds to an epitope of GRP78; wherein the L-D reagent used to make the ADC is TBLR-GGFG-DXd; and the Ab-(L-D)ₓ, wherein x is 4 or 8, comprises the structure:

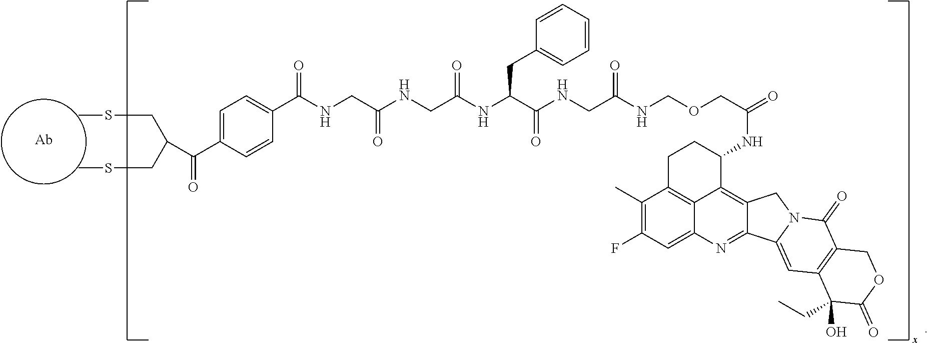

Other embodiments are also contemplated by this disclosure, as would be understood by one of skill in the art.

### Pharmaceutical Compositions and Formulations

Also provided are pharmaceutical compositions comprising any one of the antibodies, antigen-binding fragments, and immunoconjugates (e.g. antibody drug conjugates) disclosed herein. Among the compositions are pharmaceutical compositions and formulations for administration, such as for treatment, amelioration, and/or prevention of a disease or disorder. Also provided are therapeutic methods for administering the pharmaceutical compositions to subjects, e.g., patients.

The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects the choice of carrier is determined in part by the particular composition and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

In some embodiments, the formulations can include aqueous solutions. In some embodiments, the formulations can include lyophilized formulations.

The formulation or composition may also contain more than one active ingredient useful for the particular indication, disease, or condition being treated, ameliorated, and/or prevented with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as, in the case of cancer treatment, an anti-cancer drug. In some embodiments, the anti-cancer drug is a chemotherapeutic agent, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the composition in an amount effective to treat, ameliorate, and/or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. The provided compositions may be administered using standard administration techniques, formulations, and/or devices. Provided are formulations and devices, such as syringes and vials, for storage and administration of the compositions.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

Methods of Treatment, Amelioration, and/or Prevention

Also provided are methods, such as methods of treatment and uses for treating, ameliorating, and/or preventing a disease or condition, such as a cancer but also including a number of non-cancer indications, with any one of the provided antibodies (including a full-length antibody or antigen-binding fragments thereof) and immunoconjugates (e.g. antibody drug conjugates) described herein. In some embodiments, any one of the provided antibodies, and immunoconjugates (e.g. antibody drug conjugates) may be included in a composition for treating, ameliorating, and/or preventing the disease or condition in a subject in need thereof. The composition may include a pharmaceutical composition and further include a pharmaceutically acceptable carrier. A therapeutically effective amount of the pharmaceutical composition may be administered to the subject.

In some embodiments, the methods include methods of administering and uses, such as therapeutic and prophylactic uses, of any one of the antibodies and immunoconjugates (e.g. antibody drug conjugates) described herein or compositions containing the same. Such methods and uses include therapeutic methods and uses, for example, involving administration any one of the provided antibodies and immunoconjugates (e.g. antibody drug conjugates), or compositions containing the same, to a subject having a disease, condition, or disorder associated with csGRP78. Also provided herein are uses of any one of the provided antibodies and immunoconjugates (e.g. antibody drug conjugates), or compositions containing the same, in such methods and treatments, and in the preparation of a medicament in order to carry out such therapeutic methods. In some embodiments, the disease, condition, or disorder is associated with csGRP78, and/or in which cells or tissues specifically produce csGRP78.

In some embodiments, a composition containing a provided antibody or immunoconjugates (e.g. antibody drug conjugates) is administered in an effective amount to effect treatment, amelioration, and/or prevention of the disease or disorder. In some embodiments, the methods are carried out by administering any one of the provided antibodies and immunoconjugates (e.g. antibody drug conjugates), or compositions containing the same, to the subject having, having had, or suspected of having the disease or condition. In some embodiments, the methods thereby treat, ameliorate, and/or prevent the disease or condition or disorder in the subject. Also provided herein are of use of any of the compositions, such as pharmaceutical compositions provided herein, for the treatment, amelioration, and/or prevention of a disease or disorder associated with csGRP78, for example, a cancer or certain non-cancer indications.

In some embodiments, the provided antibodies and immunoconjugates (e.g. antibody drug conjugates), and compositions containing the same, can be used to treat, ameliorate, and/or prevent a cancer in which tumor cells express csGRP78 on their surface.

In some embodiments, the provided antibodies and immunoconjugates (e.g. antibody drug conjugates), and compositions containing the same, may be used to treat, ameliorate, and/or prevent different cancers.

In some embodiments, the methods may identify a subject who has, is suspected to have, or is at risk of developing a csGRP78-associated disease or disorder. Hence, provided are methods for identifying subjects with diseases or disorders associated with elevated total GRP78 expression and/or csGRP78 expression and protein placement, selecting them for treatment with any one of the provided antibodies and immunoconjugates (e.g. antibody drug conjugates) or compositions containing the same. For example, a subject may be screened for the presence of a disease or disorder associated with csGRP78, such as a csGRP78-expressing cancer. In some aspects, a sample may be obtained from a patient suspected of having a disease or disorder associated with csGRP78 expression and assayed for presence of csGRP78 in the target cells or tissue. In some aspects, a subject who tests positive for a csGRP78-associated disease or disorder may be selected for treatment by the present methods and may be administered a therapeutically effective amount of any one of the provided antibodies and immunoconjugates (e.g. antibody drug conjugates) or compositions containing the same as described herein. In some embodiments, the methods can be used to monitor the size or density of csGRP78-positive tissue, e.g. tumor, over time, e.g., before, during, or after treatment by the methods.

Compositions of this disclosure can be administered in dosages and routes and at times to be determined in appropriate pre-clinical and clinical experimentation and trials. Compositions may be administered multiple times at dosages within these ranges. Administration of the compositions may be combined with other methods useful to treat, ameliorate, and/or prevent the desired disease or condition as determined by those of skill in the art.

In some embodiments, a composition herein may contain an effective amount of a provided antibody or immunoconjugate (e.g. antibody drug conjugate) that may be from 0.001 mg to 1000 mg, such as from 0.001 mg to 100 mg, 0.001 mg to 10 mg, 0.001 mg to 1 mg, 0.001 mg to 0.1 mg or 0.001 mg to 0.01 mg. Depending on the type and severity of the disease, dosages of antibody or immunoconjugate (e.g. antibody drug conjugate) may include about 1 μg/kg to about 15 mg/kg (e.g. 0.1 mg/kg-10 mg/kg), about 1 μg/kg to about 100 mg/kg, about 0.05 mg/kg to about 10 mg/kg, about 0.5 mg/kg, about 2.0 mg/kg, about 4.0 mg/kg or about 10 mg/kg. Multiple doses may be administered intermittently, e.g. every week or every three weeks. An initial higher loading dose, followed by one or more lower doses may be administered.

In some embodiments, the antibody may be administered systemically. In additional aspects, the antibody may be administered intravenously, intradermally, intratumorally, intramuscularly, intraperitoneally, subcutaneously, or locally. The method may further comprise administering at least a second anticancer therapy to the subject. Examples of the second anticancer therapy include, but are not limited to, surgical therapy, chemotherapy, radiation therapy, cryotherapy, hormonal therapy, immunotherapy, or cytokine therapy.

In some embodiments, the treatment does not induce an immune response by the subject to the therapy, and/or does not induce such a response to a degree that prevents effective treatment of the disease or condition. In some aspects, the degree of immunogenicity and/or graft versus host response is less than that observed with a different but comparable treatment. For example, the degree of immunogenicity in some embodiments is reduced compared to a similar antibody drug conjugate including a different antibody that binds to a similar, e.g., overlapping epitope and/or that competes for binding to csGRP78 with the provided antibody, such as a reference antibody as described.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made, and equivalents may be substituted without departing from the true spirit and scope of this disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

This disclosure is further described in detail by reference to the following experimental examples. These examples are provided for purposes of illustration only and are not intended to be limiting unless otherwise specified. Thus, this disclosure should in no way be construed as being limited to the following examples, but rather, should be construed to encompass any and all variations which become evident as a result of the teaching provided herein.

Synthetic examples utilize peptide-peptide and peptide-linker coupling reagents, protecting groups and reactions that are well known to those in the art, see for example, Tarfah I, et al., J. Saudi Chem. Soc., 16, (2), pp. 97-116 (2012).

Some of the linker constructs (L), components of linker constructs, and linker-drug (L-D) constructs are commercially available and can be used in some of the syntheses described below. For example, GMP Val-Cit-PAB-MMAE:

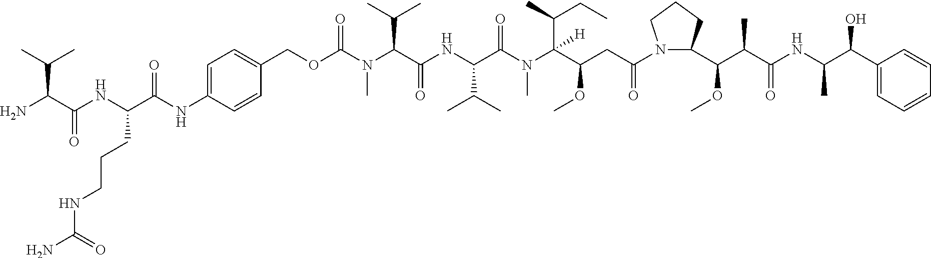 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

is available from MedChemExpress LLC, Princeton NJ, USA (wwwmedchemexpress.com)

Without further description, it is believed that one of ordinary skill in the art can, using the preceding description and the following illustrative examples, make and utilize the compounds of the present disclosure and practice the claimed methods. The following working examples, therefore, specifically point out the preferred embodiments of the present disclosure and are not to be construed as limiting in any way the remainder of the disclosure.

### Example 1

### Synthesis of TBR-[Glu-(GGFG-DXd)]₂-Glu-(amino-PEG₂₄-Me)₂

### Example 1A: Synthesis of GGFG-DXd

Fmoc-Gly-Gly-Phe-Gly-NH—CH₂—O—CH₂CO₂H (available from Broad Pharm, San Diego CA, USA) was coupled with Exatecan under standard amide formation conditions, resulting in the L-D precursor of structure (1A).

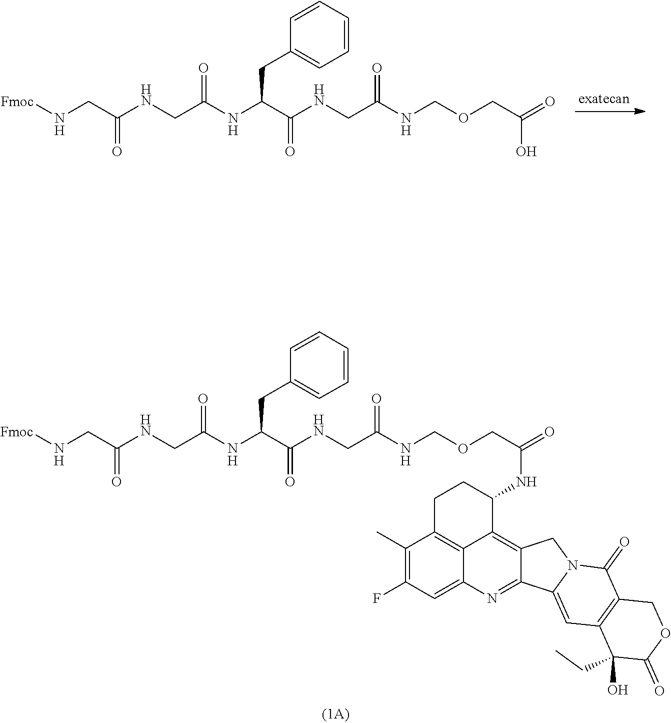

Compound (1A) was deprotected by introducing it to a solution of 20% triethylamine (TEA) in DMF to produce the L-D reagent (1B):

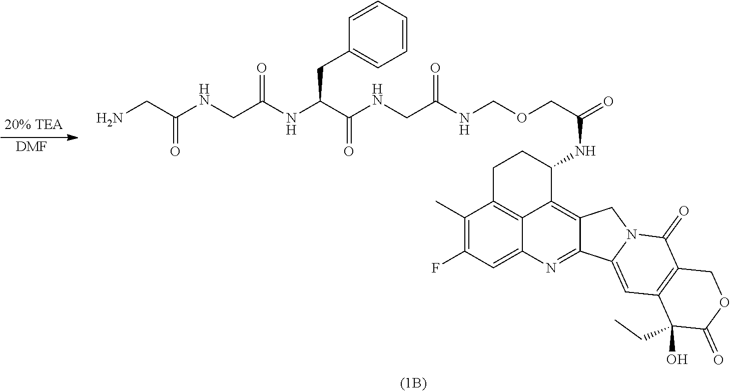

wherein Fmoc represents a 9-Fluorenyl-methyloxycarbonyl protecting group.

### Example 1B: Solid State Synthesis of Tri-Glutamic Acid Branched PEG24 Linker

Sequential solid state synthesis addition of allyloxycarbonyl protected (L) glutamic acid groups (see, for example, AD Morley, Tetrahedron Letters, Volume 41, Issue 38, 2000, pp 7401-7404) followed by addtion of one underivatized L-glutamic acid, followed by cleavage of the reaction product and then protection of the amine with fluorenylmethyloxy-carbonyl chloride (Fmoc-Cl) provided the linear tri-glutatmate (1C). As shown below, compound (1C) was then reacted with two equivalents of HO-PEG24-NH₂ to form intermediate (1D)

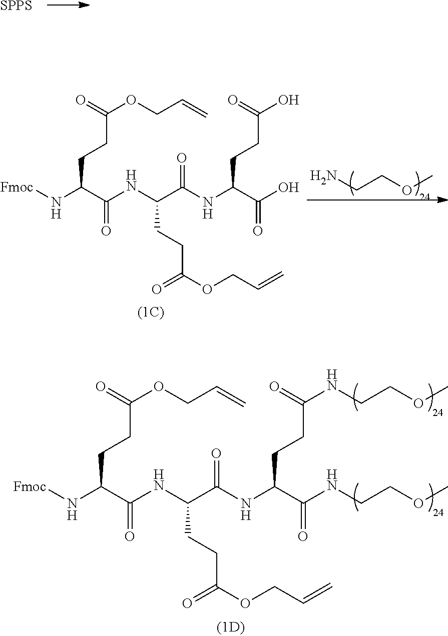

### Example 1C: Synthesis of (GGFG-DXd)₂-(PEG24)₂

Removal of the allyl protecting groups from compound (1D) to form the di-acid, followed by formation of amide bonds with GGFG-DXd (1B) and subsequent removal of the Fmoc protecting group with 20% TEA in DMF provided the [Glu-(GGFG-DXd)]₂-Glu-(amino-PEG₂₄-Me)₂ construct (1D′):

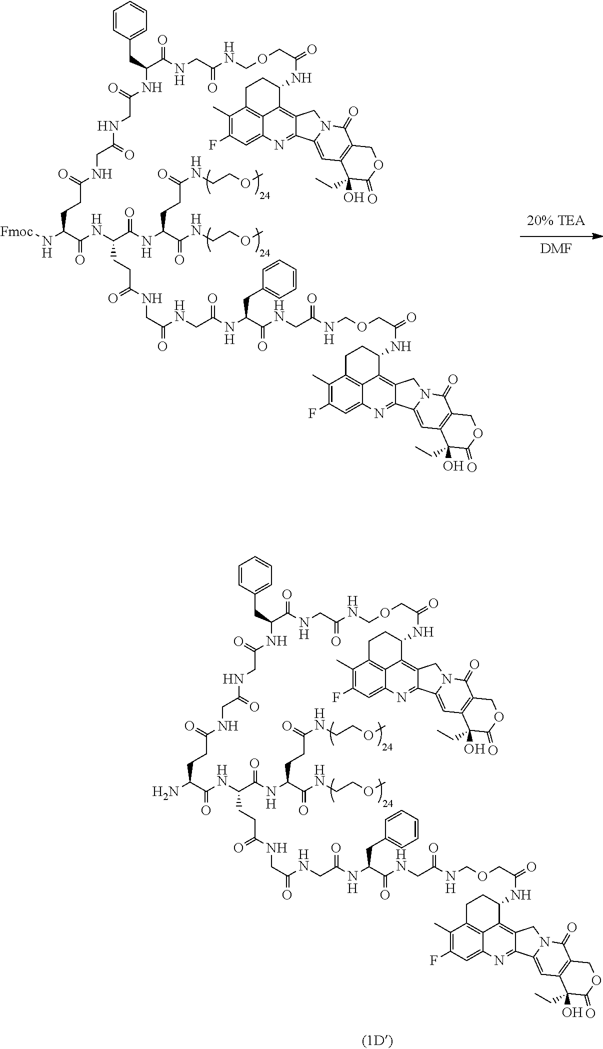

### Example 1D: Synthesis of TBR-[Glu-(GGFG-DXd)]2-Glu-(amino-PEG24-Me)₂

4-[2,2-bis[(p-tolylsulfonyl) methyl]acetyl]benzoic acid-NHS ester, (e), was prepared according to Brocchini et al., Nat. Protoc. 1 (2006) 2241-2252. Reaction of EDC (shown below) with the carboxyl group of 4-[2,2-bis[(p-tolylsulfonyl) methyl]acetyl]benzoic acid and reaction with N-hydroxysuccinimde provided the Abzena Thiobribdge (TBR) linking reagent (1E):

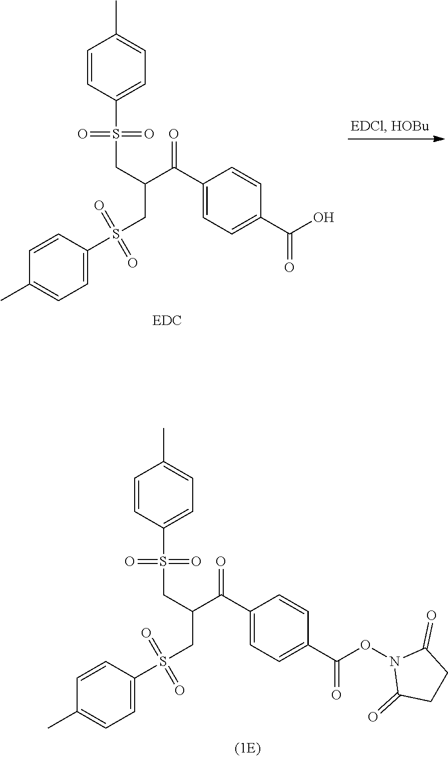 [SMILES: Cc1ccc(S(=O)(=O)CC(CS(=O)(=O)c2ccc(C)cc2)C(=O)c2ccc(C(=O)O)cc2)cc1 ; Cc1ccc(S(=O)(=O)CC(CS(=O)(=O)c2ccc(C)cc2)C(=O)c2ccc(C(=O)ON3C(=O)CCC3=O)cc2)cc1]

The coupling reaction of 4-[2,2-bis[(p-tolylsulfonyl) methyl]acetyl]benzoic acid-NHS ester (E) and [Glu-(GGFG-DXd)]₂-Glu-(amino-PEG₂₄-Me)₂ construct (1D′) provided the L-D reagent TBR-[Glu(GGFG-DXd)]₂-Glu-(amino-PEG₂₄-Me)₂, structure (1F):

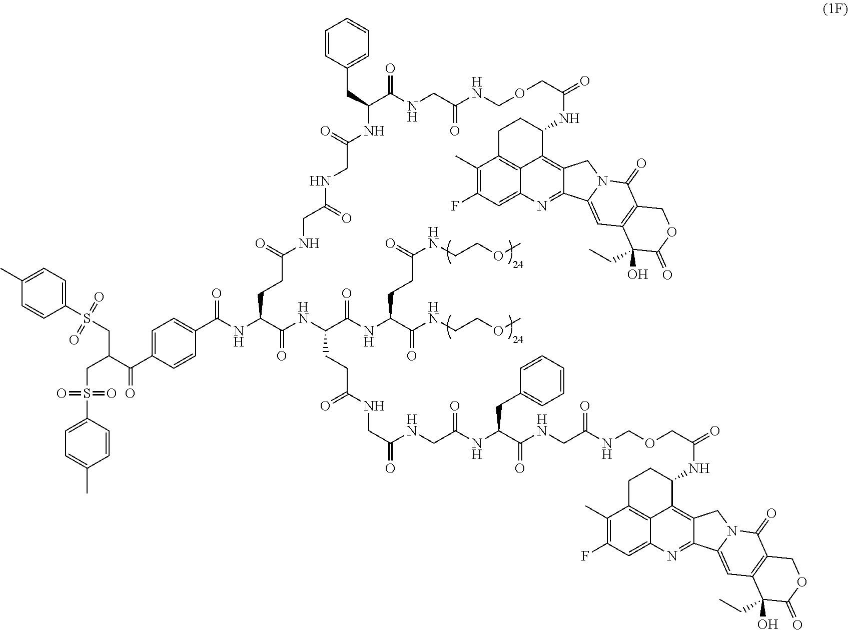

wherein Tos represents tosyl or p-toluenesulfonyl-(H₃C—C₆H₄—SO₂—).

### Example 2

### Synthesis of TBR-Glu-(Val-Cit-PAB-MMAE)-amino-PEG₂₄-Me

4-[2,2-bis[(p-tolylsulfonyl) methyl]acetyl]benzoic acid-NHS ester as described in Example 1D was reacted with 5-t-butoxy-Glu to form compound (2A):

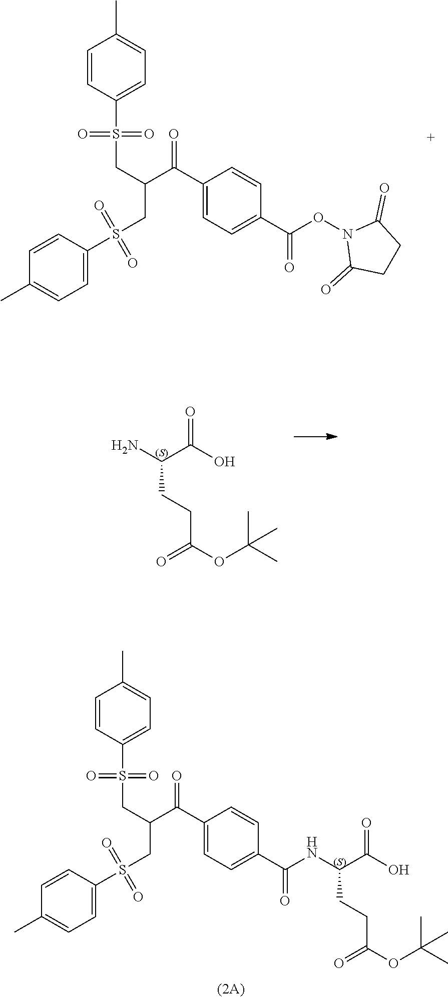

Compound (2A) was deprotected via TFA hydrolysis and then amidated with amino-PEG24-Me to form compound (2B), and subsequently coupled with Val-Cit-PAB-MMAE, using the amide conjugation reagents 1-[Bis(dimethylamino)methylene]-1H-1,2,3-triazolo[4,5-b]pyridinium 3-oxide hexafluorophosphate (HATU) and N-methylmorpholine (NMM) in dimethylformamide (DMF), to form the TBR L-D reagent (2C).

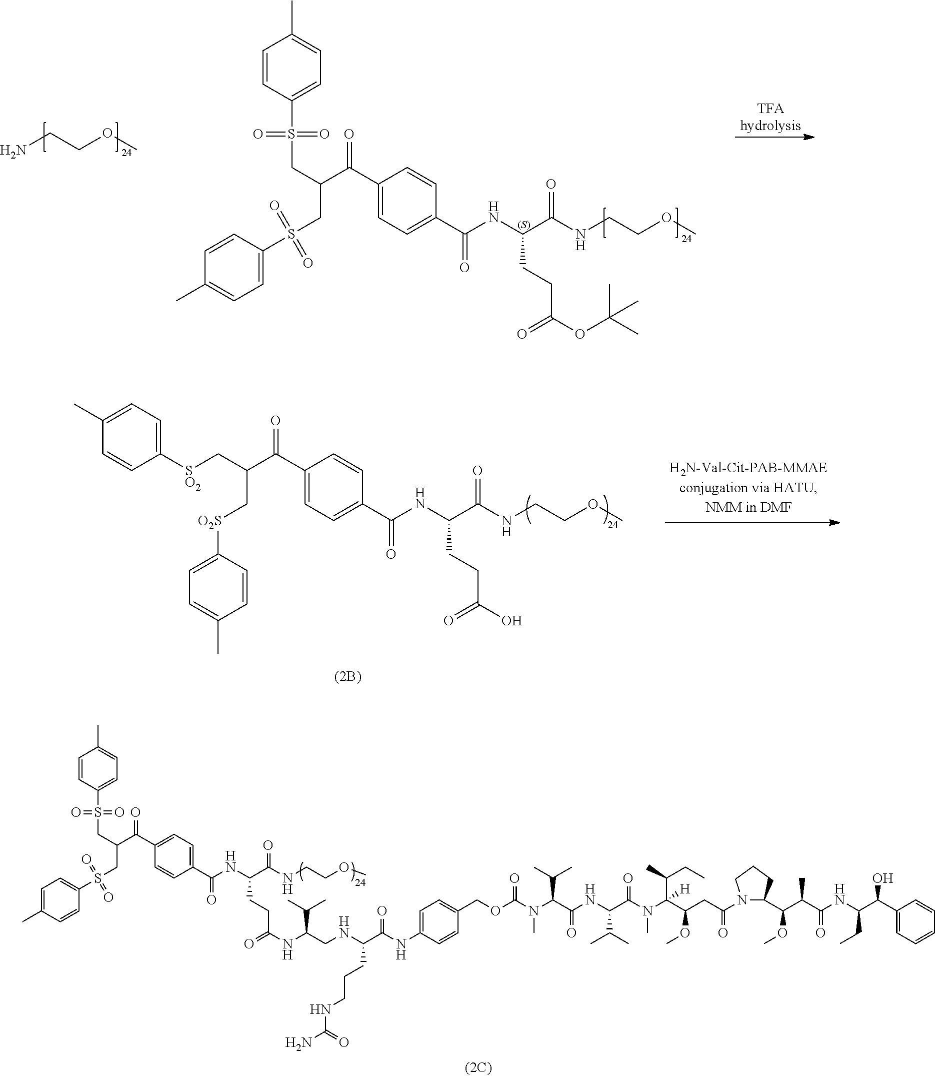

### Example 3

### Synthesis of TBR-Glu-(Val-Cit-PAB-Exatecan)-amino-PEG₂₄-Me

t-Butyloxycarbonyl-valyl-citrullyl-(4-aminobenzyl)-(4-nitrophenyl)carbonate (Boc-Val-Cit-PAB-PNP) (commercially available from Broad Pharm, San Diego CA, USA) was reacted with Exatecan in the presence of ethyldiisopropylamine (DIPEA) or triethylamine (TEA) to form the carbamate linkage as shown in the scheme below. After removal of the BOC protecting group with TFA from the Boc-Val-Cit-PAB-DXd intermediate, the intermediate was coupled with TBR-Glu-(PEG)₂₄-Me (structure 2B from Example 2) via HATU and NMM in DMF to provide the TBR L-D reagent (3A):

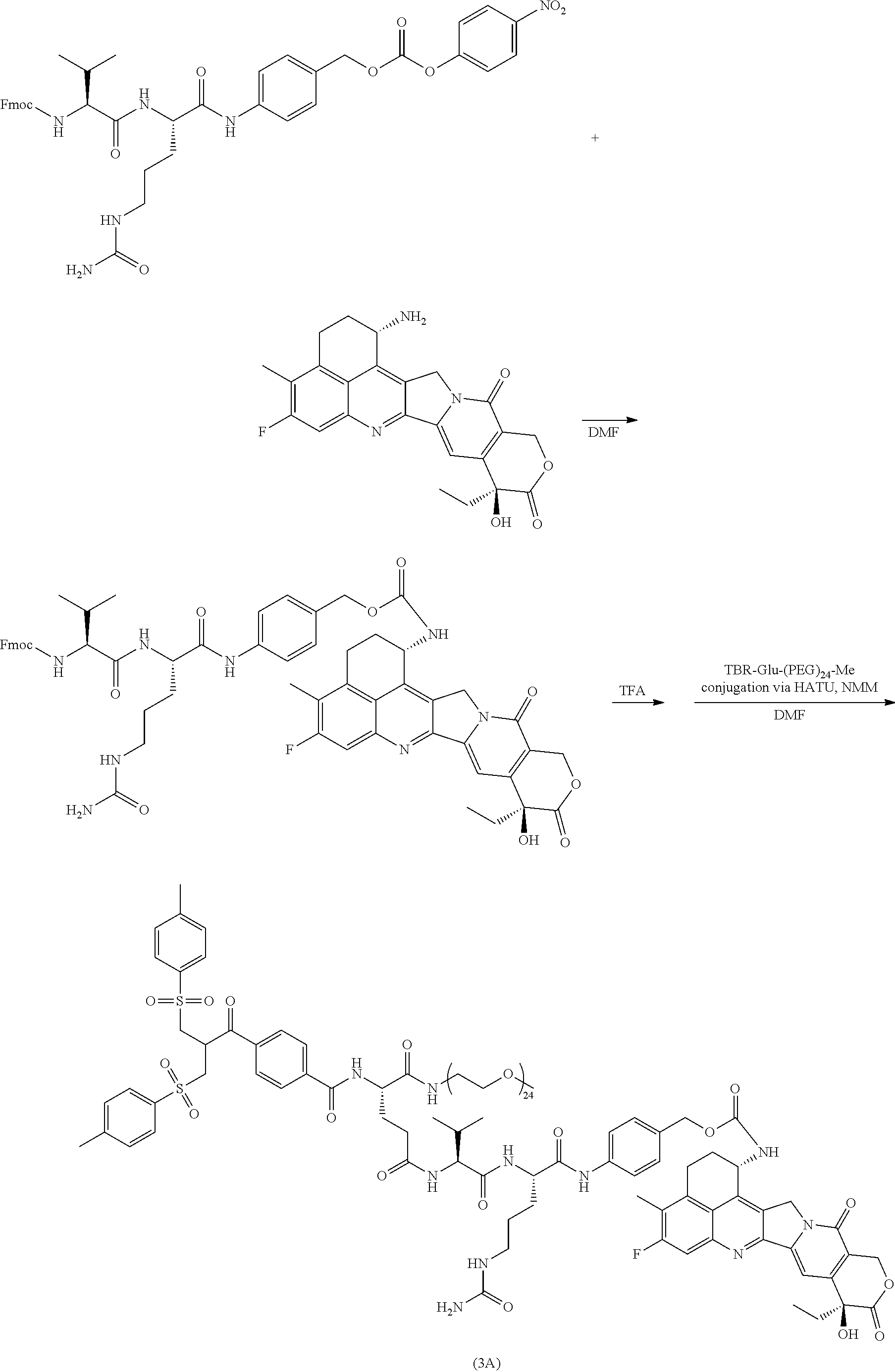

### Example 4

### Synthesis of TBR-Glu-(Val-Cit-PAB-MMAE)-amino-PEG₂₄-Me

Fmoc-Val-Cit-PAB-OH (commercially available from Broad Pharm) is reacted with bis(4-nitrophenyl) carbonate in the presence of DIEA in DMF to provide the nitrophenyl carbonate intermediate (4A):

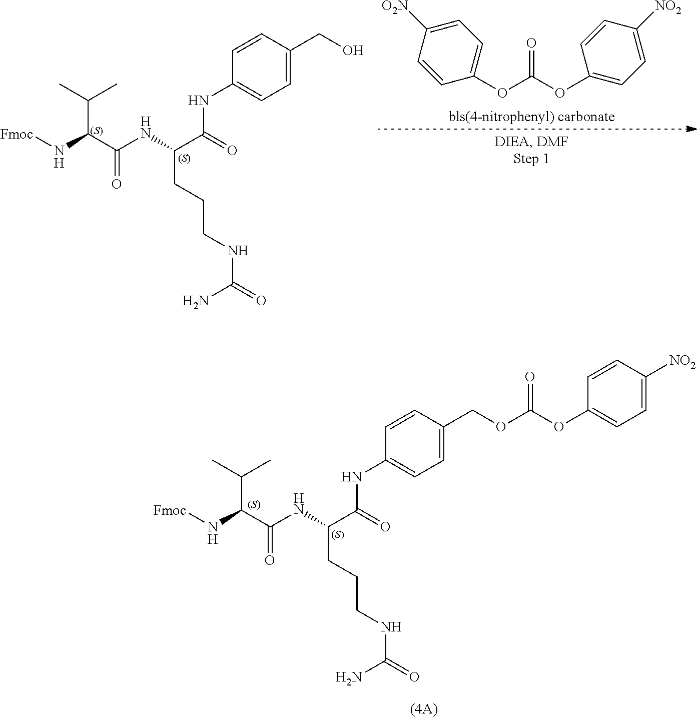

The nitrophenyl carbonate intermediate (4A) was then coupled with N-methyl amino termius of MMAE in the presence of hydroxybenzotriazole (HOBt) and N,N-diisopropylethylamine (DIEA) to form the carbamate bond linking Fmoc-Val-Cit-PAB with MMAE, (compound (4B):

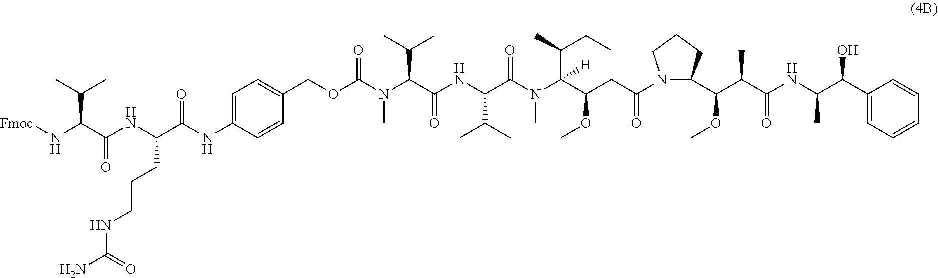

Removal of the Fmoc protecting group via an amine in a polar solvent (see for example Methods for Removing the Fmoc Group, in Pennington, M. W., Dunn, B. M. (eds) Peptide Synthesis Protocols. Methods in Molecular Biology, vol 35. Humana Press, Totowa, NJ) provided Val-Cit-PAB-MMAE (4C) for further reaction to form the TBR-Glu-(Val-Cit-PAB-MMAE)-amino-PEG₂₄-Me linking reagent (4D).

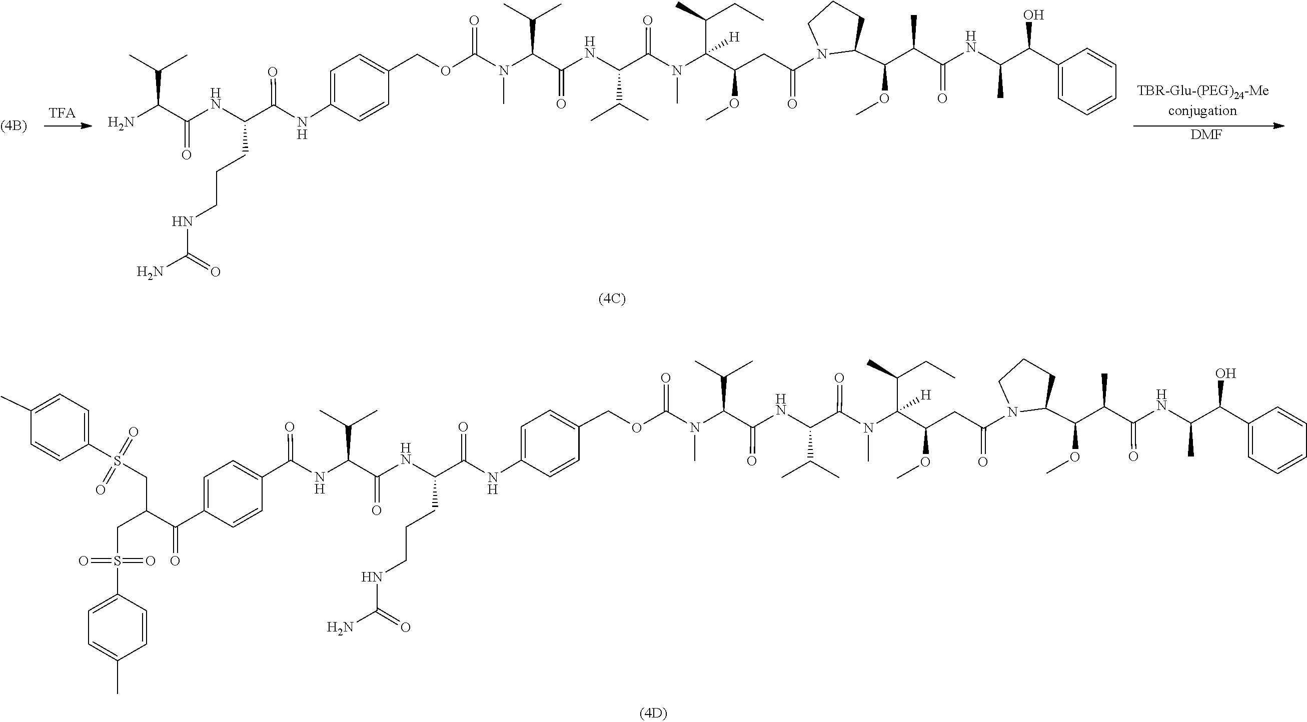

### Example 5

### Synthesis of the TBRL Linker Reagent

4-[3-(4-methylphenyl) sulfonyl-2-[(4-methylphenyl) sulfonylmethyl]propanoyl]benzoic acid (available from, for example, Broad Pharma, San Diego CA, USA) was reacted with HS-PEG7-Me in DMF and TEA to produce the disuulfide intermediate (5A) which was oxidized using oxone in 9:1 methanol to water solvent to provide the TBRL reagent (5B):

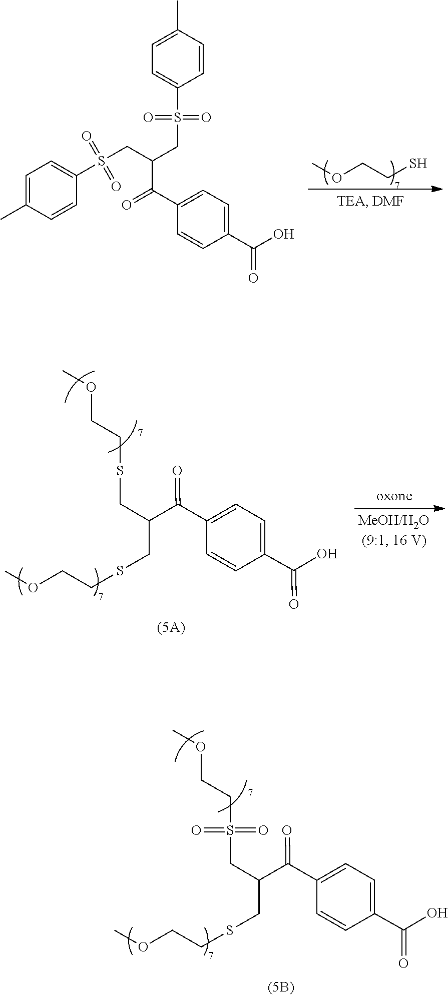

The TLBR structure was reacted with GGFG-DXd constructs as follows: (5B) was amidated with (1B) using the amide conjugation reagents N,N,N′,N′-tetramethyl-O-(benzotriazol-1-yl)uronium tetrafluoroborate (TBTU) and N-methylmorpholine (NMM) in dimethylformamide (DMF) to form TBLR-GGFG-DXd (5C). Alternative amide conjugation reagents could include but are not limited to TBTU and N,N-Diisopropylethylamine (DIEA) in DMF; hydroxybenzotriazole (HOBt), DIEA and 1-Ethyl-3-(3-dimethylaminopropyl)carbodiimide (EDCl) in DMF; and 1-[Bis(dimethylamino)methylene]-1H-1,2,3-triazolo[4,5-b]pyridinium 3-oxide hexafluorophosphate (HATU) and NMM in DMF. Similarly, (5B) was amidated with Glu-(GGFG-DXd)₂ using the amide conjugation reagents HATU and NMM in DMF to form TBLR-Glu-(GGFG-DXd)₂ (5D).

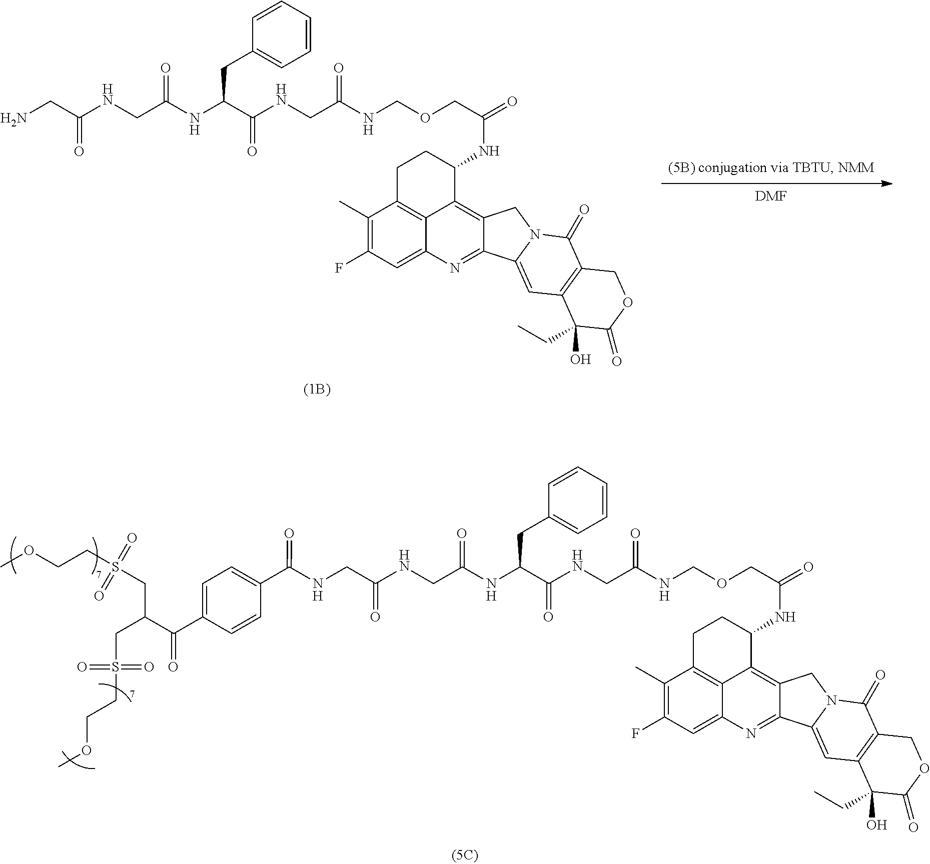

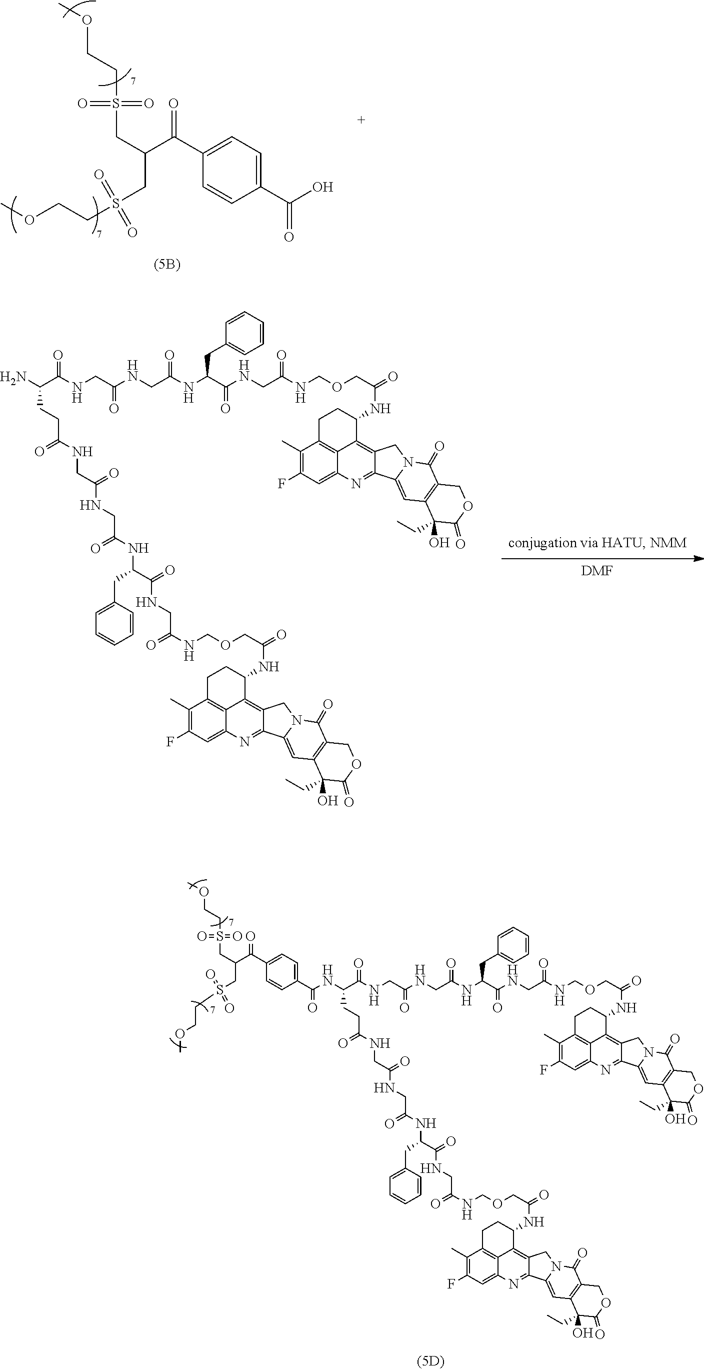

### Example 6

Conjugation of Bis-Sulfone (TBR or TBLR) Linker Reagents with Reduced Antibody

To a solution of antibody at 5 mg/mL in 20 mM sodium phosphate, pH 7.5, 150 mM NaCl and 20 mM EDTA is added a 5 mM TCEP (tris (2-carboxyethyl) phosphine) solution (18 L) and the resulting mixture is incubated at 40° C. for 1 h. The reduced antibody solution is diluted to 3.33 mg/mL with 20 mM sodium phosphate buffer, pH 7.5, 150 mM NaCl and 20 mM EDTA.

To the reduced antibody, a solution the bis-sulfone reagent of the desired L-D in DMF (at about 3.23 mg/mL) is added and the resulting solution is mixed and incubated at 22° C. for 22 h. The reaction is quenched by addition of 50 mM N-acetyl-L-cysteine (36 L) and incubated at 22° C. for a further 1 h.

### Conjugation of TBLR-GGFG-DXd to Antibody B4

To a solution of antibody at 10 mg/mL in 20 mM sodium phosphate, pH 7.8, 150 mM NaCl and 20 mM EDTA is added 6 molar equivalents of TCEP (tris (2-carboxyethyl) phosphine) solution and the resulting mixture incubated at 20° C. for 2 h. To the reduced antibody solution is added 4.9 molar equivalents of TBLR-GGFG-DXd in dimethylacetamide (DMA) sufficeint to achieve 10% DMA in the conjugation reaction. The resulting mixture was incubated at 20° C. for 20 h and then quenched by addition of 8 molar equivalents of N-acetyl-L-cysteine and incubated at 20° C. for a further 30 min. The resulting ADC (B4-TB-GGFG-DXd) from the quenched conjugation reaction was then purified by hydrophobic interaction chromatography. Reaction time and purification conditions are controlled to achieve an average DAR of 4.

Further antibody reduction and bis-sulfone linker conjugation reaction conditions are disclosed in US Pat. Appln. No. 2021/0093733, which is incorporated herein in its entirety.

### Example 7

### Manufacture of ADCs

Anti-GRP78 ADCs were prepared from antibodies B4 and F6 according to the procedures essentially as outlined in Example 6. Antibody B4 comprises SEQ ID NO:21 and SEQ ID NO:23, and antibody F6 comprises SEQ ID NO:22 and SEQ ID NO:24. The B4 and F6 antibodies were manufactured as generally set forth in WO2023215768A2, which is incorporated herein in its entirety and for all purposes.

Three cytotoxic payloads were tested with these antibodies: 1) monomethyl auristatin E (MMAE), an auristatin inhibiting polymerization of microtubules; 2) Exatecan, a first DNA topoisomerase I inhibitor; and 3) DXd, a second DNA topoisomerase I inhibitor. ADCs were prepared with the proprietary ThioBridge® conjugation technology from Abzena (Cambridge, UK), a disulfide rebridging conjugation technology. The B4 and F6 mAbs were conjugated to TBR-Glu-(Val-Cit-PAB-MMAE)-amino-PEG₂₄-Me; TBR-Glu-(Val-Cit-PAB-Exatecan)-amino-PEG₂₄-Me; and TBLR-GGFG-DXd.

### Example 8

### Analytical Results for Manufactured ADCs

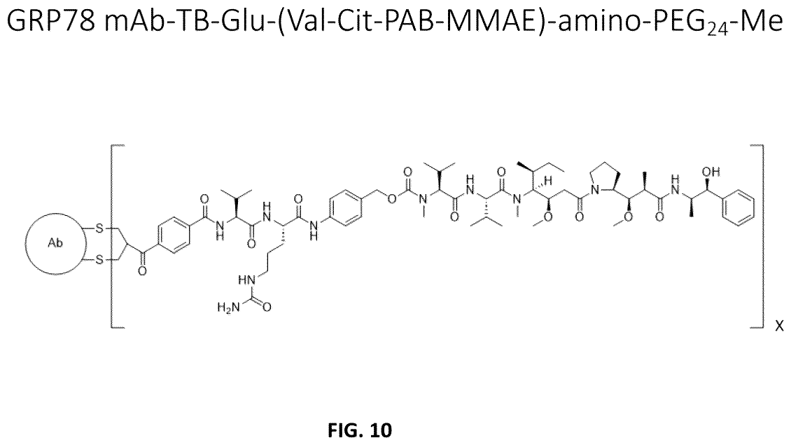

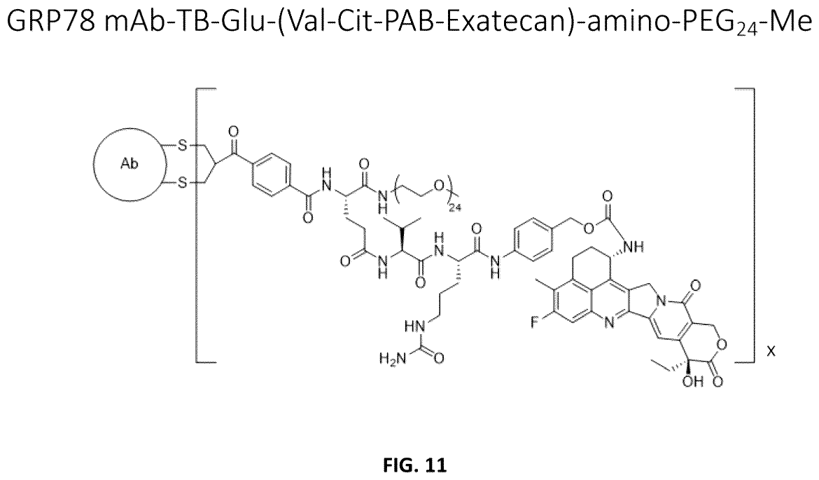

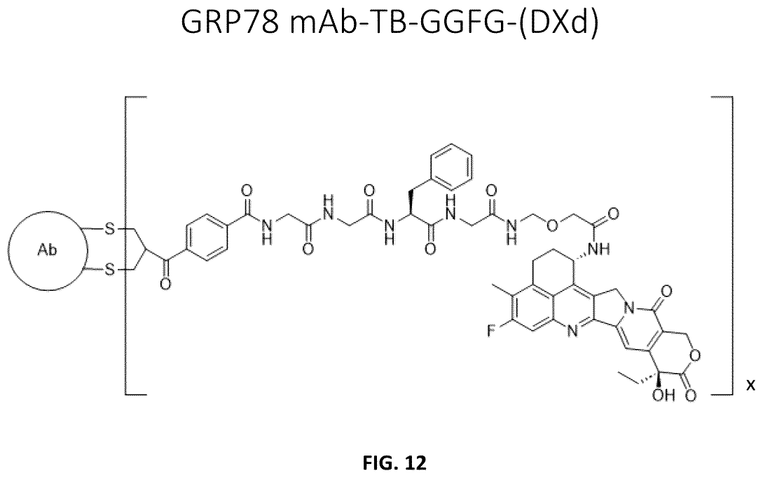

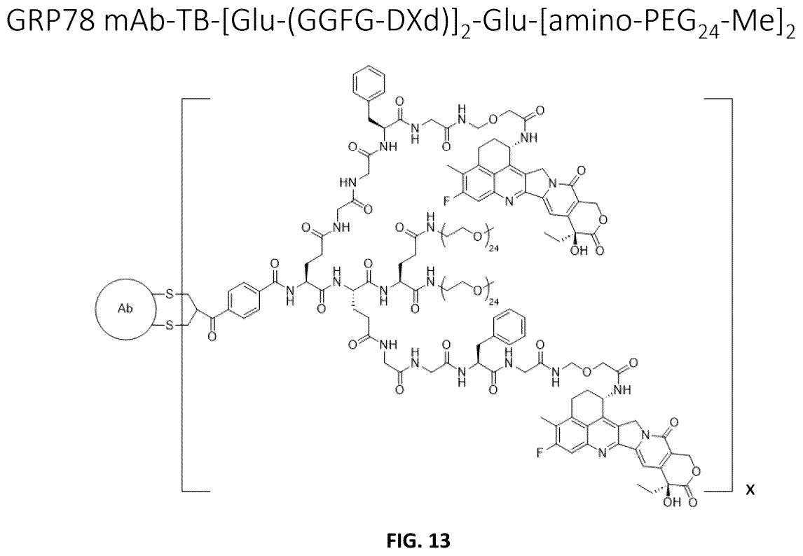

It is to be noted that ThioBridge (TBR) L-D reagents were used for conjugation in these examples to form (Ab)-TB-Glu-(Val-Cit-PAB-MMAE)-amino-PEG₂₄-Me (ave. DAR4) as shown in FIG. 10, (Ab)-TB-Glu-(Val-Cit-PAB-Exatecan)-amino-PEG₂₄-Me (ave. DAR4) as shown in FIG. 11, and (Ab)-TB-GGFG-DXd (ave. DAR4) as shown in FIG. 12 as well as a DAR8 version. The TBR-[Glu(GGFG-DXd)]₂-Glu-(amino-PEG₂₄-Me)₂linker format was designed with dual branched 24-unit PEG polymers to reduce aggregation propensity and improve aqueous solubility as shown in FIG. 13.

Concentration and average DAR determination by UV absorbance: The concentration and average DAR of the conjugates were determined by UV absorbance at 280 nm (A280) and at a wavelength λ corresponding to a maximum of absorbance for the linker-payload (AX) using a Nanodrop 2000 spectrophotometer. Measurements were taken in triplicate and the average values used for calculations. Molar extinction coefficients used for antibodies and linker-payloads were determined experimentally (see Example 8A, below).
Endotoxin level determination method: An EndoSafe portable test sstem (PTS) (Charles River) was used to determine the level of endotoxin. EndoSafe-PTS is a chromogenic kinetic test system aligned with USP <85> and Pharm Eur 2.6.14 that provides quantitative Limulus Amebocyte Lysate (LAL) results. The EndoSafe-PTS utilizes LAL reagents in FDA-licensed disposable test cartridges which are pre-loaded with all the reagents required to perform a LAL test. The EndoSafe-PTS mimics licensed LAL kinetic chromogenic methodology by measuring colour intensity directly related to the endotoxin concentration in a sample; the concentrations are calculated against an internal standard curve (0.01-1.00 EU/mL) associated with the lot number of the cartridges.

### Example 8A: Analytical Results

The analytical results of the generated conjugates are summarized below:

B4-TB-Glu-(Val-Cit-PAB-MMAE)-amino-PEG₂₄-Me

| Analysis | Results |
|---|---|
| Sample Name | B4-TB-Glu-(Val-Cit-PAB- |
| | MMAE)-amino-PEG₂₄-Me |
| Batch Code | 001 |
| Appearance | Clear colorless solution |
| Identity (LC-MS) | MW confirmed |
| Average DAR (LC-MS) | Average DAR: 4.0 |
| % Purity (SEC) | 99.9% monomeric |
| % free reagent related species | Not detected |
| Endotoxin (EU/mg) | 0.00 |

F6-TB-Glu-(Val-Cit-PAB-MMAE)-amino-PEG₂₄-Me

| Analysis | Results |
|---|---|
| Sample Name | F6-TB-Glu-(Val-Cit-PAB- |
| | MMAE)-amino-PEG₂₄-Me |
| Batch Code | 002 |
| Appearance | Clear colorless solution |
| Identity (LC-MS) | MW confirmed |
| Average DAR (LC-MS) | Average DAR: 4.0 |
| % Purity (SEC) | 99.9% monomeric |
| % free reagent related species | Not detected |
| Endotoxin (EU/mg) | 0.00 |

B4-TB-Glu-(Val-Cit-PAB-Exatecan)-amino-PEG₂₄-Me

| Analysis | Results |
|---|---|
| Sample Name | B4-TB-Glu-(Val-Cit-PAB- |
| | Exatecan)-amino-PEG₂₄-Me |
| Batch Code | 003 |
| Appearance | Clear colorless solution |
| Identity (LC-MS) | MW confirmed |
| Average DAR (LC-MS) | Average DAR: 4.0 |
| % Purity (SEC) | 99.9% monomeric |
| % free reagent related species | Not detected |
| Endotoxin (EU/mg) | 0.00 |

F6-TB-Glu-(Val-Cit-PAB-Exatecan)-amino-PEG₂₄-Me

| Analysis | Results |
|---|---|
| Sample Name | F6-TB-Glu-(Val-Cit-PAB- |
| | Exatecan)-amino-PEG₂₄-Me |
| Batch Code | 004 |
| Appearance | Clear colorless solution |
| Identity (LC-MS) | MW confirmed |
| Average DAR (LC-MS) | Average DAR: 4.0 |
| % Purity (SEC) | 99.9% monomeric |
| % free reagent related species | Not detected |
| Endotoxin (EU/mg) | 0.00 |

B4-TB-GGFG-DXd

| Analysis | Results |
|---|---|
| Sample Name | B4-TB-GGFG-DXd |
| Batch Code | 005 |
| Appearance | Clear colorless solution |
| Identity (LC-MS) | MW confirmed |
| Average DAR (LC-MS) | Average DAR: 4.0 |
| % Purity (SEC) | 99.9% monomeric |
| % free reagent related species | Not detected |
| Endotoxin (EU/mg) | 0.00 |

F6-TB-GGFG-DXd

| Analysis | Results |
|---|---|
| Sample Name | F6-TB-GGFG-DXd |
| Batch Code | 006 |
| Appearance | Clear colorless solution |
| Identity (LC-MS) | MW confirmed |
| Average DAR (LC-MS) | Average DAR: 4.0 |
| % Purity (SEC) | 99.9% monomeric |
| % free reagent related species | Not detected |
| Endotoxin (EU/mg) | 0.00 |

B4-TB-[Glu(GGFG-DXd)]₂-Glu-(amino-PEG₂₄-Me)₂

| Analysis | Results |
|---|---|
| Sample Name | B4-TB-[Glu(GGFG-DXd)]₂- |
| | Glu-(amino-PEG₂₄-Me)₂ |
| Batch Code | 007 |
| Appearance | Clear colorless solution |
| Identity (LC-MS) | MW confirmed |
| Average DAR (LC-MS) | Average DAR: 4.0 |
| % Purity (SEC) | 99.9% monomeric |
| % free reagent related species | Not detected |
| Endotoxin (EU/mg) | 0.00 |

B4-TB-[Glu(GGFG-DXd)]₂-Glu-(amino-PEG₂₄-Me)₂

| Analysis | Results |
|---|---|
| Sample Name | B4-TB-[Glu(GGFG-DXd)]₂- |
| | Glu-(amino-PEG₂₄-Me)₂ |
| Batch Code | 008 |
| Appearance | Clear colorless solution |
| Identity (LC-MS) | MW confirmed |
| Average DAR (LC-MS) | Average DAR: 4.0 |
| % Purity (SEC) | 99.9% monomeric |
| % free reagent related species | Not detected |
| Endotoxin (EU/mg) | 0.00 |

ADCs were successfully conjugated resulting in an average DAR of 4.0 for all the conjugates as determined by LC-MS. The average DAR was 4.0 to 4.1 as determined by HIC-HPLC.

No significant presence of reagent related species could be detected in the ADCs comparing the SEC trace of the samples to the SEC trace of the formulation buffer in the low molecular weight species region at different wavelengths.

LAL assay also confirmed the low levels of endotoxins in all ADC samples (<0.1 EU/mg). LC-MS intact mass analysis confirmed the identity of the conjugated species.

Expected profiles were observed for the ADCs by SDS-PAGE, with presence of interchain disulfide bridged conjugated species including intact antibody conjugate, fragment species HHL and HH and of ‘half-antibody’ fragment species HL. High levels (64-73%) of inter heavy-chains bridging were observed for the ThioBridge®-MMAE and -SN38 ADCs.

All ADCs were isolated following purification by preparative HIC and in moderate yields (33-54% recovery).

### Example 9

### In Vivo Anti-Tumor Activity of ADCs Directed to GRP78 in a Patient-Derived Xenograft Model of Triple Negative Breast Cancer

Patient-derived tumor xenografts (PDXs), in which tumor fragments are surgically dissected from cancer patients and directly transplanted into immunodeficient mice, have been established as predictive models in translational research. PDX susceptibility to anti-cancer drugs has been closely correlated with clinical data in patients from whom PDX models have been derived, and PDX models often maintain the cellular and histopathological structures of the original tumors. Genetic analysis of tumor cells from PDX models following transplantation has shown preservation of the genomic and gene expression profiles between PDXs and parental patient tumors and sensitivity to standard chemotherapeutics in PDXs closely correlates with clinical data in patients from which the PDXs are derived. Over 500 different PDX models have been characterized for 25 different tumor types in multi-center efforts, which has provided comprehensive information on the correlation between these models and various tumor types. PDX models are thus highly effective in vivo models for predicting the efficacy of anti-cancer therapeutics, especially for solid tumors.

Tumor regression studies were performed using a single-dose strategy in vivo, using female NSG™ mice implanted with a PDX model of triple negative breast cancer, TM00098 (BR1126F), purchased from Jackson Laboratories (Bar Harbor, ME). These mice are extremely immunodeficient and carry two mutations: severe combined immune deficiency (scid) and a complete null allele of the IL2 receptor common gamma chain (IL2rg^{null}). The scid mutation is a mutation in the DNA repair complex protein Prkdc and renders the mice B and T cell deficient. The IL2rg^{null} mutation prevents cytokine signaling through multiple receptors, leading to a deficiency in functional NK cells. This severe immunodeficiency allows the implantation of patient derived xenografts (PDX) and growth of human tissue-based tumors in the mice. These tumors have similar characteristics to the human tumors from which they are derived (e.g., gene expression) and are predictive of human response to therapeutic agents.

The initial and final diagnosis of the tumor from which the TM00098 PDX model was derived was invasive ductal carcinoma, AJCC IA/Grade 3. The primary site of human tumor was breast, and the sample site of the tumor for the PDX model was the primary site of the tumor. The tumor used for the TM00098 PDX model was treatment naïve. The patient from which the sample was derived was a 64-year-old white, non-Hispanic female. The sample was obtained by surgical resection. The host strain of the sample for engraftment was NOD.Cg-Prkdcscid Il2rgtm1Wjl/SzJ (aka NSG or NOD Scid gamma).

Mice received one treatment dose of test articles at 1 mg/kg, 2 mg/kg or 4 mg/kg via tail vein injection. The number of mice used per group was n=5. Measurements were performed 3 times weekly with the aid of a digital caliper (Fisherbrand™ Traceable™ Digital Carbon Fiber Calipers). All studies included a control group of equal number of mice to those in the treatment group. Control groups received PBS tail vein injections and were submitted to the same handling procedures as mice treated with test articles. First treatment was administered when tumors reached ˜200 mm³ volume.

Treatment arms for the in vivo study were assigned for both the GRP78 B4 IgG and GRP78 F6 IgG constructs and were dosed as follows: 1) (Ab)-TB-Glu-(Val-Cit-PAB-MMAE)-amino-PEG₂₄-Me (FIG. 10)-1 mg/kg, 2 mg/kg or 4 mg/kg; 2) (Ab)-TB-Glu-(Val-Cit-PAB-Exatecan)-amino-PEG₂₄-Me (FIG. 11)-1 mg/kg, 2 mg/kg or 4 mg/kg; and 3) (Ab)-TB-GGFG-DXd (FIG. 12)-1 mg/kg, 2 mg/kg or 4 mg/kg. Additionally, unconjugated antibodies were dosed as follows: F6 IgG unconjugated (4 mg/kg); and B4 IgG unconjugated (4 mg/kg). A total of 105 mice were administered a single treatment dose in each arm of the study.

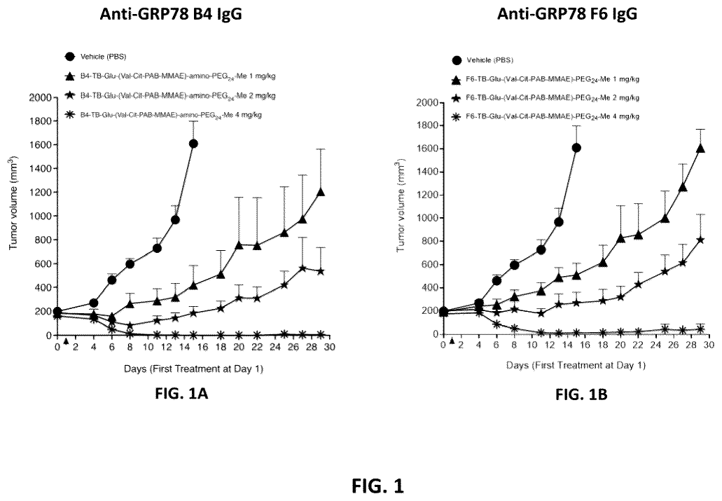

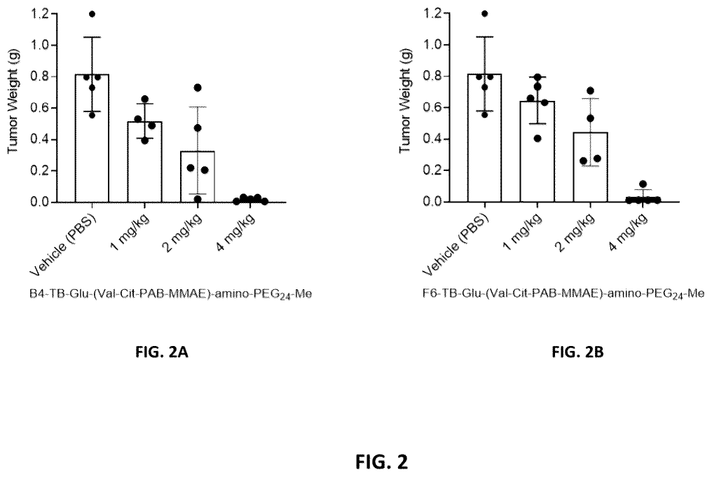

The B4-TB-Glu-(Val-Cit-PAB-MMAE)-amino-PEG₂₄-Me and F6-TB-Glu-(Val-Cit-PAB-MMAE)-amino-PEG₂₄-Me study arms showed partial regression in the 1 mg/kg and 2 mg/kg dosage groups (n=4), and full regression from a single dose in the 4 mg/kg dosage groups (n=4) (FIG. 1 and Table 5). The B4-TB-Glu-(Val-Cit-PAB-MMAE)-amino-PEG₂₄-Me dosage group did have one subject that began showing tumor regrowth at Day 29. The tumor weights at 30 days following this single administration showed a dose-dependent shrinkage (FIG. 2).

TABLE 5

| | Day 0 | Day 4 | Day 6 | Day 8 | Day 10 | Day 13 | Day 15 | Day 18 | Day 20 | Day 22 | Day 25 | Day 27 | Day 29 |
|---|---|---|---|---|---|---|---|---|---|---|---|---|---|
| (1) | 198.23 | 269.97 | 461.09 | 596.67 | 815.28 | 967.54 | 1608.89 | | | | | | |
| (2) | 182.04 | 175.63 | 157.83 | 264.31 | 281.54 | 318.68 | 419.08 | 512.61 | 758.26 | 752.61 | 862.24 | 975.75 | 1205.96 |
| (3) | 186.15 | 165.03 | 108.60 | 107.04 | 120.36 | 142.38 | 184.33 | 223.65 | 311.76 | 309.31 | 420.54 | 638.83 | 536.46 |
| (4) | 176.73 | 131.94 | 48.83 | 13.67 | 10.78 | 2.20 | 0.80 | 1.00 | 1.00 | 1.00 | 9.87 | 6.20 | 6.20 |
| (5) | 193.39 | 241.19 | 254.26 | 325.78 | 376.59 | 490.48 | 511.29 | 623.06 | 830.11 | 858.81 | 1002.13 | 1272.45 | 1607.30 |
| (6) | 202.99 | 212.04 | 184.19 | 214.83 | 178.32 | 256.77 | 271.03 | 288.96 | 322.02 | 428.61 | 541.38 | 617.05 | 812.37 |
| (7) | 174.74 | 183.93 | 85.47 | 50.72 | 15.93 | 12.33 | 15.26 | 16.94 | 21.14 | 24.76 | 43.69 | 36.50 | 44.66 |

(1) Vehicle
(2) B4-TB-Glu-(Val-Cit-PAB-MMAE)-amino-PEG₂₄-Me 1 mg/kg
(3) B4-TB-Glu-(Val-Cit-PAB-MMAE)-amino-PEG₂₄-Me 2 mg/kg
(4) B4-TB-Glu-(Val-Cit-PAB-MMAE)-amino-PEG₂₄-Me 4 mg/kg
(5) F6-TB-Glu-(Val-Cit-PAB-MMAE)-amino-PEG₂₄-Me 1 mg/kg
(6) F6-TB-Glu-(Val-Cit-PAB-MMAE)-amino-PEG₂₄-Me 2 mg/kg
(7) F6-TB-Glu-(Val-Cit-PAB-MMAE)-amino-PEG₂₄-Me 4 mg/kg

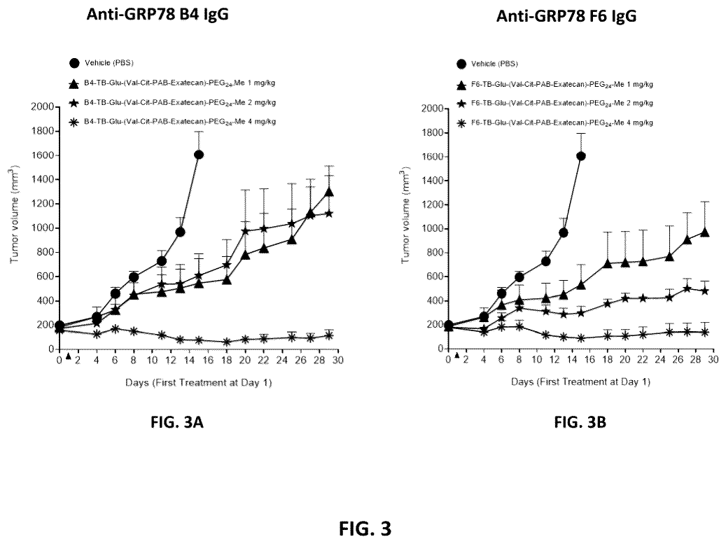

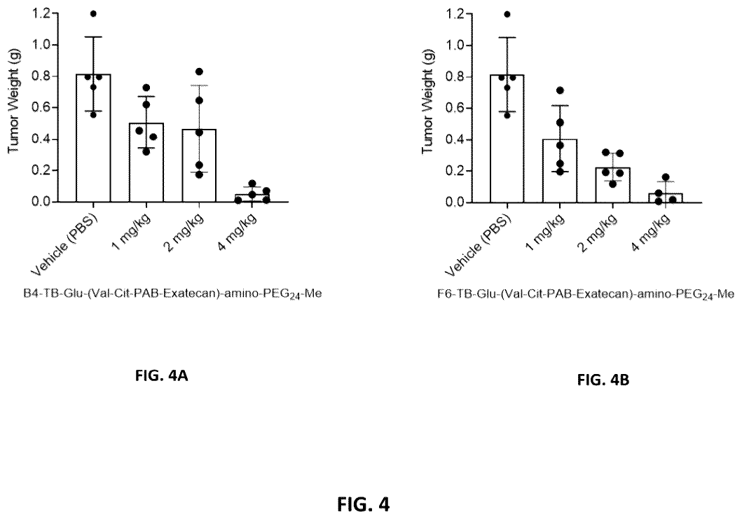

The B4-TB-Glu-(Val-Cit-PAB-Exatecan)-amino-PEG₂₄-Me and F6-TB-Glu-(Val-Cit-PAB-Exatecan)-amino-PEG₂₄-Me study arms showed partial regression in the 1 mg/kg, 2 mg/kg and 4 mg/kg dosage groups (n=4), with the regression following a dose-dependent reduction in tumor volume groups (n=4) (FIGS. 3 and 4, and Table 6).

TABLE 6

| | Day 0 | Day 4 | Day 6 | Day 8 | Day 10 | Day 13 | Day 15 | Day 18 | Day 20 | Day 22 | Day 25 | Day 27 | Day 29 |
|---|---|---|---|---|---|---|---|---|---|---|---|---|---|
| (1) | 198.23 | 269.97 | 461.09 | 596.67 | 815.28 | 967.54 | 1608.89 | | | | | | |
| (2) | 182.63 | 268.96 | 325.97 | 454.00 | 475.84 | 503.98 | 545.77 | 576.11 | 782.07 | 836.08 | 907.52 | 1130.01 | 1303.02 |
| (3) | 171.97 | 243.14 | 328.41 | 445.19 | 537.58 | 541.19 | 608.84 | 696.41 | 974.72 | 995.08 | 1036.79 | 1102.88 | 1120.51 |
| (4) | 171.77 | 126.65 | 169.79 | 150.06 | 118.95 | 81.28 | 77.44 | 62.47 | 83.91 | 87.57 | 99.65 | 93.27 | 115.59 |
| (5) | 186.34 | 263.63 | 366.49 | 365.87 | 423.66 | 452.17 | 534.38 | 711.50 | 720.09 | 728.34 | 770.33 | 911.72 | 974.33 |
| (6) | 177.99 | 166.84 | 257.48 | 339.16 | 311.84 | 287.11 | 298.45 | 375.52 | 420.86 | 421.88 | 427.09 | 501.84 | 480.55 |
| (7) | 178.19 | 138.99 | 181.18 | 184.03 | 115.76 | 99.42 | 89.64 | 106.84 | 107.83 | 119.38 | 138.75 | 142.47 | 138.58 |

(1) Vehicle
(2) B4-TB-Glu-(Val-Cit-PAB-Exatecan)-amino-PEG₂₄-Me 1 mg/kg
(3) B4-TB-Glu-(Val-Cit-PAB-Exatecan)-amino-PEG₂₄-Me 2 mg/kg
(4) B4-TB-Glu-(Val-Cit-PAB-Exatecan)-amino-PEG₂₄-Me 4 mg/kg
(5) F6-TB-Glu-(Val-Cit-PAB-Exatecan)-amino-PEG₂₄-Me 1 mg/kg
(6) F6-TB-Glu-(Val-Cit-PAB-Exatecan)-amino-PEG₂₄-Me 2 mg/kg
(7) F6-TB-Glu-(Val-Cit-PAB-Exatecan)-amino-PEG₂₄-Me 4 mg/kg

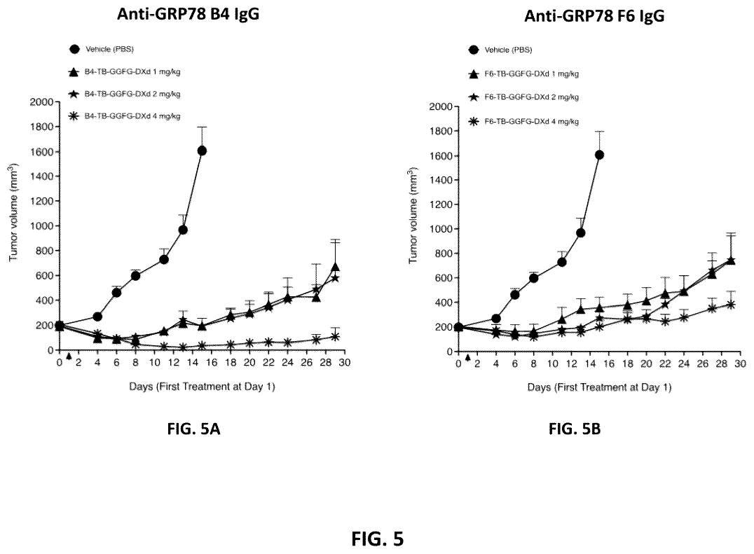

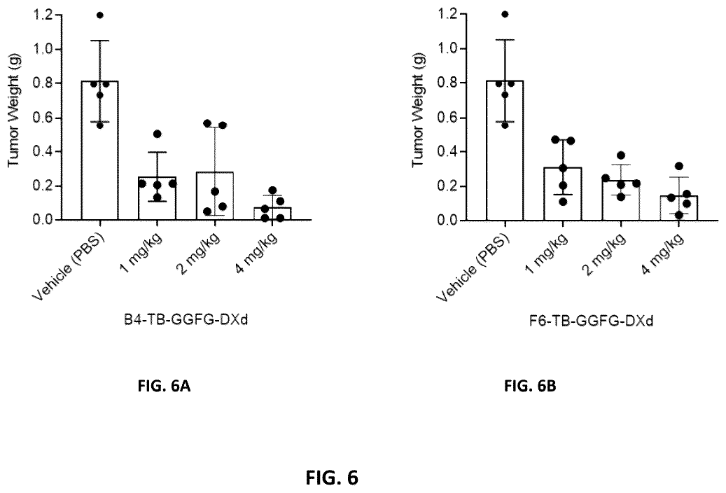

The B4-TB-GGFG-DXd and F6-TB-GGFG-DXd study arms showed partial regression in the 1 mg/kg, 2 mg/kg and 4 mg/kg dosage groups (n=4), with the regression following a dose-dependent reduction in tumor volume groups, except for the 1 mg/kg and 2 mg/kg arms in the B4-TB-GGFG-DXd dosage group which showed approximately comparable results (FIGS. 5 and 6, and Table 7).

TABLE 7

| | Day 0 | Day 4 | Day 6 | Day 8 | Day 10 | Day 13 | Day 15 | Day 18 | Day 20 | Day 22 | Day 25 | Day 27 | Day 29 |
|---|---|---|---|---|---|---|---|---|---|---|---|---|---|
| (1) | 198.23 | 269.97 | 461.09 | 596.67 | 815.28 | 967.54 | 1608.89 | | | | | | |
| (2) | 190.91 | 96.06 | 89.41 | 83.88 | 156.44 | 215.82 | 196.11 | 285.28 | 306.77 | 366.39 | 428.83 | 425.85 | 674.01 |
| (3) | 193.20 | 108.25 | 91.34 | 108.82 | 144.66 | 246.06 | 192.55 | 257.45 | 291.54 | 343.23 | 404.79 | 488.39 | 579.84 |
| (4) | 204.86 | 132.76 | 89.75 | 46.72 | 29.50 | 24.05 | 36.06 | 43.78 | 56.77 | 64.34 | 60.79 | 82.12 | 107.52 |
| (5) | 204.19 | 173.15 | 162.39 | 164.94 | 264.12 | 344.93 | 358.72 | 380.05 | 413.73 | 475.70 | 490.16 | 630.60 | 744.56 |
| (6) | 197.05 | 138.47 | 120.75 | 144.39 | 180.69 | 195.19 | 274.07 | 263.21 | 289.49 | 383.38 | 492.65 | 663.25 | 748.07 |
| (7) | 195.25 | 167.16 | 136.86 | 117.94 | 154.70 | 155.00 | 200.38 | 262.74 | 266.40 | 244.57 | 276.65 | 350.78 | 382.05 |

(1) Vehicle
(2) B4-TB--GGFG-DXd 1 mg/kg
(3) B4-TB-GGFG-DXd 2 mg/kg
(4) B4-TB-GGFG-DXd 4 mg/kg
(5) F6-TB-GGFG-DXd 1 mg/kg
(6) F6-TB-GGFG-DXd 2 mg/kg
(7) F6-TB-GGFG-DXd 4 mg/kg

### Example 10

### In Vivo Anti-Tumor Activity of ADCs Directed to GRP78 in a Patient-Derived and a Cell Line-Derived Xenograft Model of Castration-Resistant Prostate Cancer

Tumor regression studies were also performed in vivo using a prostate cancer model MDA-PCa-118b from MD Anderson (Houston, Texas) using a multi-dose administration strategy. The initial and final diagnosis of the tumor from which this model was derived was of metastatic castration-resistant prostate cancer. Treatment arms for the in vivo study were dosed as follows: 1) B4-TB-Glu-(Val-Cit-PAB-MMAE)-amino-PEG₂₄-Me (FIG. 10) at 1 mg/kg, 2 mg/kg or 4 mg/kg; 2) B4-TB-GGFG-DXd (FIG. 12) at 1 mg/kg, 2 mg/kg or 4 mg/kg; and

3) B4-TB-[Glu(GGFG-DXd)]₂-Glu-(amino-PEG₂₄-Me)₂ (FIG. 13) 1 mg/kg, 2 mg/kg or 4 mg/kg.

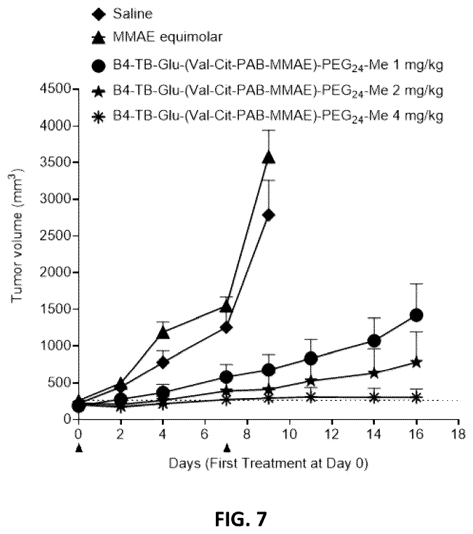

A total of two treatments in each study arm were administered at day 0 and day 7. Measurements were made 3 times weekly over a period of 16 days. B4-TB-Glu-(Val-Cit-PAB-MMAE)-amino-PEG₂₄-Me study arm showed partial regression in the 1 mg/kg and 2 mg/kg dosage groups (n=4), and sustained regression in the 4 mg/kg dosage groups (n=4), with the regression following a dose-dependent reduction (FIG. 7 and Table 8).

TABLE 8

| | Day 0 | Day 2 | Day 4 | Day 7 | Day 9 | Day 11 | Day 14 | Day 16 |
|---|---|---|---|---|---|---|---|---|
| (1) | 215.68 | 440.21 | 782.09 | 1258.21 | 2786.71 | | | |
| (2) | 257.92 | 496.13 | 1191.54 | 1548.03 | 3577.52 | | | |
| (3) | 183.50 | 274.42 | 367.69 | 580.92 | 679.20 | 836.04 | 1073.36 | 1423.11 |
| (4) | 222.52 | 203.58 | 259.31 | 389.63 | 410.37 | 526.90 | 634.53 | 785.31 |
| (5) | 200.03 | 174.47 | 213.73 | 270.68 | 289.99 | 305.30 | 299.86 | 300.11 |

(1) Saline
(2) MMAE equimolar
(3) B4-TB-Glu-(Val-Cit-PAB-MMAE)-PEG24-Me 1 mg/kg
(4) B4-TB-Glu-(Val-Cit-PAB-MMAE)-PEG24-Me 2 mg/kg
(5) B4-TB-Glu-(Val-Cit-PAB-MMAE)-PEG24-Me 4 mg/kg

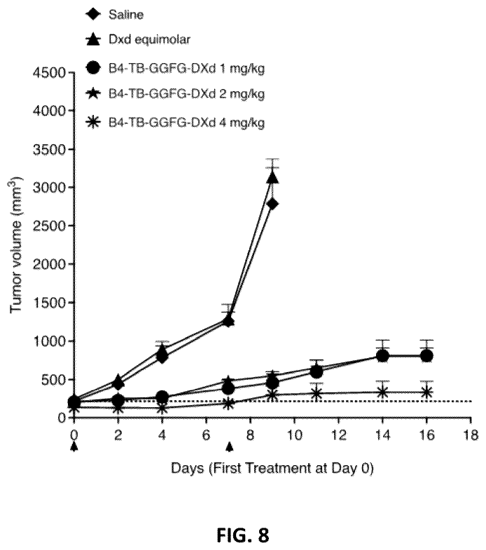

The B4-TB-GGFG-DXd study arm showed partial regression in the 1 mg/kg and 2 mg/kg and sustained regression in the 4 mg/kg dosage groups (n=4), with the 1 mg/kg and 2 mg/kg dosage groups showing essentially equivalent regression. (FIG. 8 and Table 9).

TABLE 9

| | Day 0 | Day 2 | Day 4 | Day 7 | Day 9 | Day 11 | Day 14 | Day 16 |
|---|---|---|---|---|---|---|---|---|
| (1) | 215.68 | 440.21 | 782.09 | 1258.21 | 2786.71 | | | |
| (2) | 245.64 | 500.00 | 890.43 | 1286.68 | 3138.05 | | | |
| (3) | 208.04 | 226.33 | 274.92 | 385.28 | 459.30 | 599.41 | 808.80 | 808.80 |
| (4) | 201.91 | 255.96 | 253.58 | 484.90 | 549.28 | 649.22 | 800.51 | 800.51 |
| (5) | 180.02 | 172.35 | 202.88 | 286.82 | 299.49 | 318.01 | 336.60 | 336.60 |

(1) Saline
(2) DXd equimolar
(3) B4-TB-GGFG-DXd 1 mg/kg
(4) B4-TB-GGFG-DXd 2 mg/kg
(5) B4-TB-GGFG-DXd 4 mg/kg

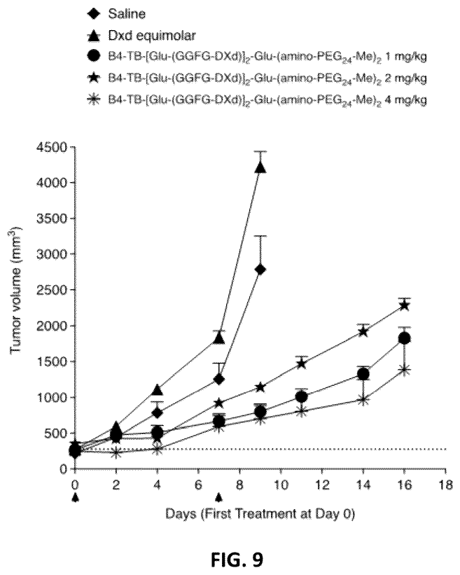

The B4-TB-[Glu-(GGFG-DXd)]₂-Glu-(amino-PEG₂₄-Me)₂ study arm showed partial regression in all three dosage groups (1 mg/kg, 2 mg/kg and 4 mg/kg) (n=4) (FIG. 9 and Table 10).

TABLE 10

| | Day 0 | Day 2 | Day 4 | Day 7 | Day 9 | Day 11 | Day 14 | Day 16 |
|---|---|---|---|---|---|---|---|---|
| (1) | 215.68 | 440.21 | 782.09 | 1258.21 | 2786.71 | | | |
| (2) | 271.24 | 595.29 | 1117.84 | 1836.43 | 4222.23 | | | |
| (3) | 264.86 | 474.08 | 510.64 | 664.48 | 798.69 | 1010.78 | 1328.00 | 1828.49 |
| (4) | 352.93 | 426.59 | 429.04 | 923.46 | 1148.56 | 1470.71 | 1917.96 | 2287.31 |
| (5) | 245.41 | 226.80 | 276.05 | 591.71 | 699.53 | 805.74 | 968.40 | 1384.56 |

(1) Saline
(2) DXd8 equimolar
(3) B4-TB-[Glu(GGFG-DXd)]₂-Glu-(amino-PEG₂₄-Me)₂1 mg/kg
(4) B4-TB-[Glu(GGFG-DXd)]₂-Glu-(amino-PEG₂₄-Me)₂2 mg/kg
(5) B4-TB-[Glu(GGFG-DXd)]₂-Glu-(amino-PEG₂₄-Me)₂4 mg/kg

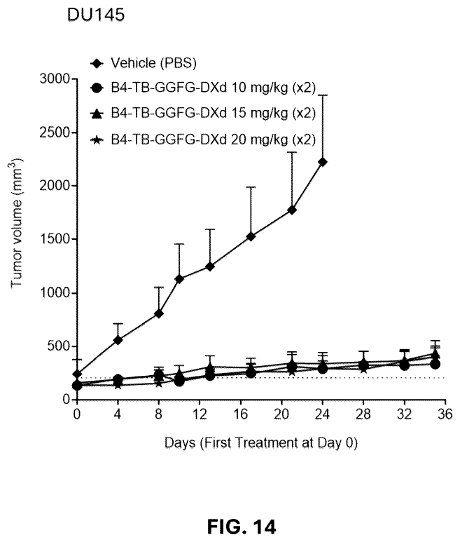

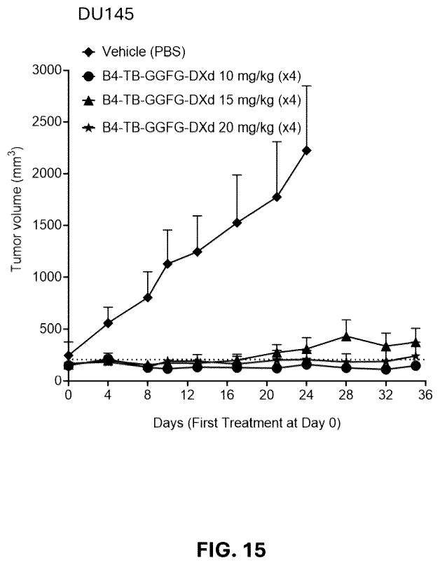

Tumor regression studies were also performed with the DU145 prostate cancer cell line. This cell line was isolated from the brain of a patient with prostate cancer and does not have prostate antigen and is not hormone sensitive. Treatment arms for the in vivo study were B4-TB-GGFG-DXd dosed at 10, 15, or 20 mg/kg with two different schedules: two doses at days 0 and 7 or four doses at days 0, 4, 7 and 11. All treatment arms showed partial regressions and some complete regressions with the four-dose schedule (FIGS. 14 and 15, and Table 11).

TABLE 11

| | Day 0 | Day 4 | Day 8 | Day 10 | Day 13 | Day 17 | Day 21 | Day 24 | Day 28 | Day 32 | Day 35 |
|---|---|---|---|---|---|---|---|---|---|---|---|
| (1) | 244.48 | 557.98 | 806.51 | 1128.46 | 1245.37 | 1526.13 | 1774.50 | 2224.57 | | | |
| (2) | 134.22 | 196.51 | 236.17 | 171.33 | 227.11 | 250.10 | 313.66 | 293.73 | 326.81 | 324.28 | 337.33 |
| (3) | 158.82 | 192.63 | 227.74 | 251.58 | 311.37 | 304.10 | 344.82 | 339.64 | 354.05 | 366.78 | 433.72 |
| (4) | 136.19 | 136.70 | 154.49 | 188.83 | 236.86 | 267.98 | 265.55 | 295.40 | 289.31 | 361.34 | 399.30 |
| (5) | 152.49 | 213.86 | 131.41 | 121.81 | 136.16 | 131.51 | 126.76 | 162.30 | 129.86 | 115.26 | 150.29 |
| (6) | 162.99 | 199.10 | 158.97 | 175.26 | 173.69 | 199.71 | 272.99 | 308.09 | 433.09 | 336.12 | 373.81 |
| (7) | 171.17 | 183.93 | 136.72 | 193.01 | 192.78 | 166.11 | 202.71 | 206.84 | 185.03 | 189.78 | 238.94 |

(1) Saline
(2) B4-TB-GGFG-DXd 10 mg/kg (x2)
(3) B4-TB-GGFG-DXd 15 mg/kg (x2)
(4) B4-TB-GGFG-DXd 20 mg/kg (x2)
(5) B4-TB-GGFG-DXd 10 mg/kg (x4)
(6) B4-TB-GGFG-DXd 15 mg/kg (x4)
(7) B4-TB-GGFG-DXd 20 mg/kg (x4)

### Example 11

### In Vivo Anti-Tumor Activity of ADCs Directed to GRP78 in Patient-Derived Xenograft Models of Colorectal Cancer

Tumor regression studies were performed in vivo using two PDX models of colorectal cancer: TM00375 and TM00980 purchased from Jackson Laboratories.

TM00375 has the following characteristics. The diagnosis of the patient from which the model was derived was metastatic colon adenocarcinoma, stage IV. The primary site of human tumor was the colon, and the sample site of the tumor for the PDX model was the muscle metastatic site. The patient from which the sample was derived was a 59-year-old female, with race and ethnicity unreported and smoker status unreported. The sample was obtained by surgical biopsy.

TM00980 has the following characteristics. The diagnosis of the patient from which the model was derived was colon carcinoma, stage IV. The primary site of human tumor was the colon, and the sample site of the tumor for the PDX model was the colon. The patient from which the sample was derived was a 60-year-old male, with race and ethnicity unreported and smoker status unreported. The sample was obtained by an unknown procedure.

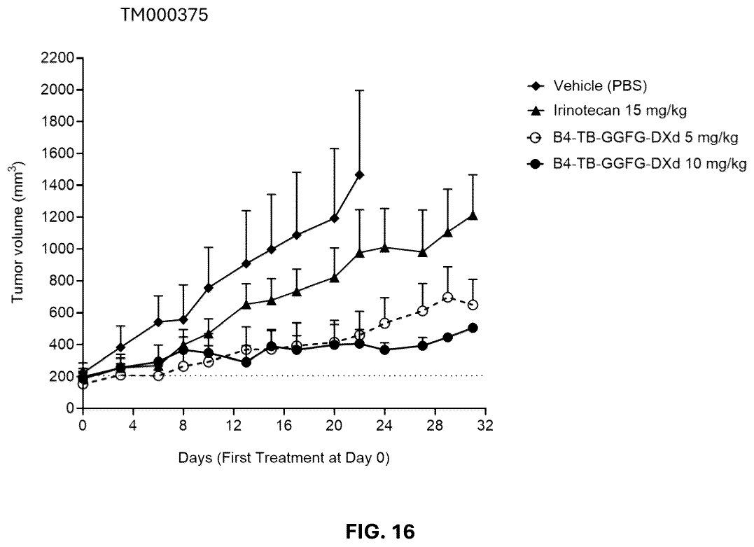

Treatment arms for the in vivo study were irinotecan (via intraperitoneal injection) and B4-TB-GGFG-DXd (via tail vein injection) dosed at 5 or 10 mg/kg weekly. For TM000375, weekly administration of B4-TB-GGFG-DXd at both doses resulted in tumor growth delay without complete regressions while weekly administration of 15 mg/kg irinotecan resulted in a slight delay in tumor growth as compared to PBS control (FIG. 16 and Table 12).

TABLE 12

| | Day 0 | Day 3 | Day 6 | Day 8 | Day 10 | Day 13 | Day 15 | Day 17 | Day 20 | Day 22 | Day 24 | Day 27 | Day 29 | Day 31 |
|---|---|---|---|---|---|---|---|---|---|---|---|---|---|---|
| (1) | 226.11 | 382.88 | 541.47 | 556.30 | 756.67 | 907.96 | 996.83 | 1087.28 | 1192.38 | 1465.30 | | | | |
| (2) | 201.90 | 256.83 | 270.06 | 398.70 | 470.18 | 653.44 | 679.54 | 735.87 | 821.23 | 976.80 | 1010.08 | 981.51 | 1106.46 | 1213.48 |
| (3) | 153.71 | 210.50 | 205.75 | 266.50 | 293.28 | 369.28 | 370.28 | 393.21 | 414.90 | 459.30 | 534.61 | 611.93 | 697.71 | 649.39 |
| (4) | 187.42 | 258.16 | 293.04 | 366.45 | 347.29 | 290.44 | 390.73 | 366.42 | 398.62 | 406.16 | 366.35 | 392.90 | 446.43 | 506.76 |

(1) Saline
(2) Irinotecan 15 mg/kg
(3) B4-TB-GGFG-DXd 5 mg/kg
(4) B4-TB-GGFG-DXd 10 mg/kg

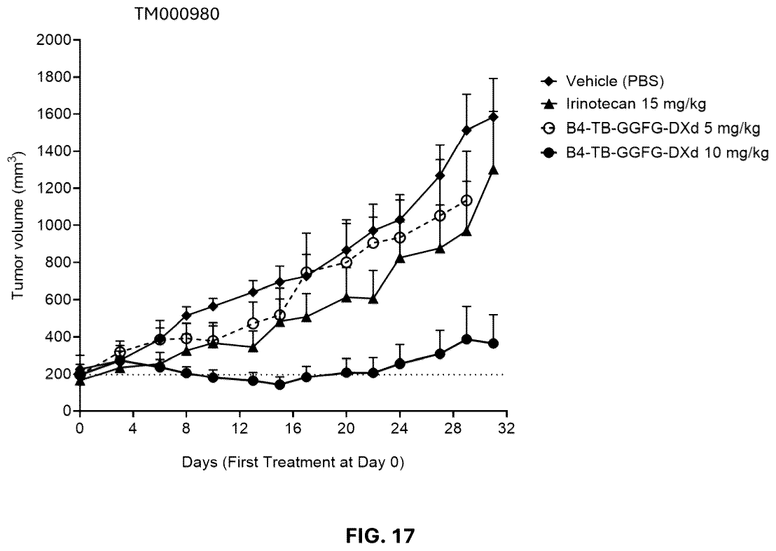

For TM000980, weekly administration of B4-TB-GGFG-DXd resulted in dose-dependent tumor growth delay: 5 mg/kg resulted in a slight delay in tumor growth as compared to PBS control and 10 mg/kg resulted in tumor growth delay without complete regressions. Weekly administration of 15 mg/kg irinotecan resulted in a slight delay in tumor growth as compared to PBS control (FIG. 17 and Table 13).

TABLE 13

| | Day 0 | Day 3 | Day 6 | Day 8 | Day 10 | Day 13 | Day 15 | Day 17 | Day 20 | Day 22 | Day 24 | Day 27 | Day 29 | Day 31 |
|---|---|---|---|---|---|---|---|---|---|---|---|---|---|---|
| (1) | 226.76 | 272.45 | 386.56 | 513.61 | 563.90 | 641.23 | 694.59 | 725.99 | 866.05 | 971.08 | 1029.58 | 1268.23 | 1514.01 | 1583.86 |
| (2) | 165.95 | 234.87 | 255.89 | 324.84 | 366.02 | 344.44 | 482.46 | 506.77 | 613.64 | 606.65 | 825.11 | 877.07 | 970.41 | 1302.20 |
| (3) | 197.67 | 316.68 | 384.77 | 391.86 | 378.11 | 471.84 | 516.45 | 746.69 | 800.20 | 904.80 | 933.55 | 1051.57 | 1134.39 | |
| (4) | 196.90 | 273.10 | 238.02 | 205.90 | 181.74 | 164.68 | 142.13 | 183.42 | 208.37 | 206.61 | 255.43 | 307.56 | 387.10 | 363.99 |

(1) Saline
(2) Irinotecan 15 mg/kg
(3) B4-TB-GGFG-DXd 5 mg/kg
(4) B4-TB-GGFG-DXd 10 mg/kg

### OTHER EMBODIMENTS

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of this disclosure. The appended claims are intended to be construed to include all such embodiments and equivalent variations.

What is claimed is:

1. An immunoconjugate having the formula Ab-(L-D)x, wherein:
a. Ab is an antibody or antigen-binding fragment;
b. each L is independently a linker;
c. each D is independently a cytotoxic drug; and
d. x is an integer from 1 to 8;
wherein the antibody comprises:
i. a heavy chain variable region (VH) comprising complementarity-determining regions (CDRs) 1, 2 and 3 as set forth in SEQ ID NOS: 5, 6, and 7, or as set forth in SEQ ID NOS: 11, 12, and 13; and,
ii. a light chain variable region (VL) comprising complementarity-determining regions 1, 2 and 3 as set forth in SEQ ID NOS: 8, 9, and 10, or as set forth in SEQ ID NOS: 14, 15, and 16.

2. The immunoconjugate of claim 1, wherein:
a. the VH has at least 85%, at least 95% or 100% sequence identity to SEQ ID NO: 1, or to SEQ ID NO: 2; and
b. the VL has at least 85%, at least 95% or 100% sequence identity to SEQ ID NO: 3, or to SEQ ID NO: 4.

3. The immunoconjugate of claim 2, wherein the heavy chain constant region is as set forth in SEQ ID NO: 17, or wherein the light chain constant region is as set forth in SEQ ID NO: 18.

4. An immunoconjugate having the formula Ab-(L-D)x, wherein:
a. Ab is an antibody or antigen-binding fragment;
b. each L is independently a linker;
c. each D is independently a cytotoxic drug; and
d. x is an integer from 1 to 8;
wherein the antibody comprises:
i. a heavy chain antibody sequence as set forth in SEQ ID NO: 21 or SEQ ID NO: 22; and
ii. a light chain antibody sequence as set forth in SEQ ID NO: 23 or SEQ ID NO: 24.

5. An immunoconjugate having the formula Ab-(L-D)x, wherein:
a. Ab is an antibody or antigen-binding fragment comprising the sequence set forth in SEQ ID NO: 19 or SEQ ID NO: 20;
b. each L is independently a linker;
c. each D is independently a cytotoxic drug; and
d. x is an integer from 1 to 8.

6. The immunoconjugate of claim 1, wherein the linker is a cleavable linker selected from a cathepsin-cleavable linker and other cleavable linkers.

7. The immunoconjugate of claim 6 wherein the cleavable linker comprises a valine-citrulline (Val-Cit), wherein the cleavable linker comprises the peptide GGFG, or wherein the linker is a pH cleavable linker.

8. The immunoconjugate of claim 7, wherein the linker further comprises a maleimidocaproyl (MC) moiety, a p-aminobenzyloxycarbonyl (PAB) moiety, or both an MC and a PAB moiety.

9. The immunoconjugate of claim 1, comprising the structure: 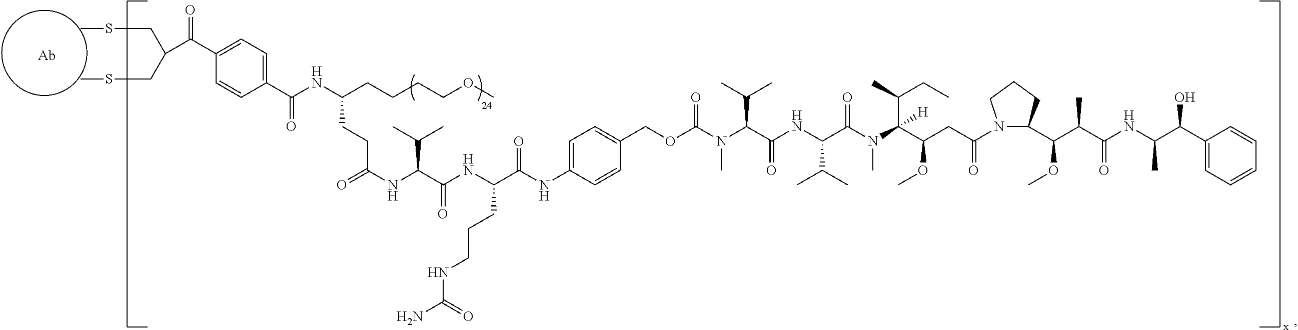
where x is selected from about 1, about 2, about 3, about 4, about 5, about 6, about 7 and about 8.

10. The immunoconjugate of claim 1, comprising the structure: 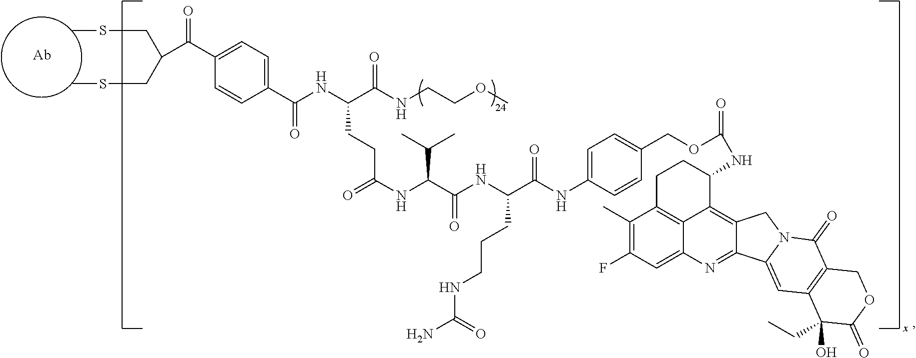
where x is selected from about 1, about 2, about 3, about 4, about 5, about 6, about 7 and about 8.

11. The immunoconjugate of claim 1, comprising the structure: 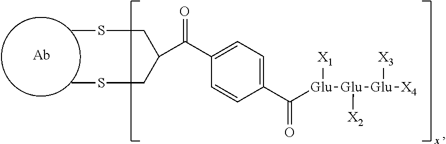
wherein Glu is L-glutamate;
X₁ is a linker-drug moiety chosen from -Val-Cit-PAB-D and -GGFG-D or is H;
X₂ is a linker-drug moiety chosen from -Val-Cit-PAB-D and -GGFG-D or is H;
X₃ is —NH-(PEG)₂₄-Me or H;
X₄ is —NH-(PEG)₂₄-Me or H;
each D is a cytotoxin independently selected from the group consisting of MMAE, Exatecan, DXd and SN38;
at least one of X₁ and X₂ is not H;
at least one of X₃ and X₄ is not H;
a horizontal line represents a bond to the carbonyl group of a glutamate side chain; and
x is selected from about 1, about 2, about 3, about 4, about 5, about 6, about 7 and about 8.

12. The immunoconjugate of claim 1, comprising the structure: 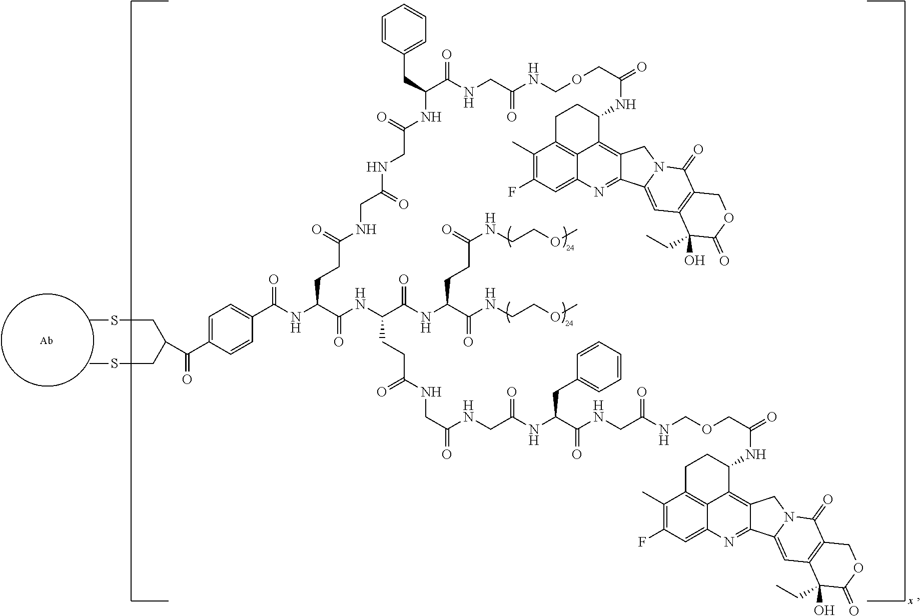
where x is selected from about 1, about 2, about 3, about 4, about 5, about 6, about 7 and about 8.

13. The immunoconjugate of claim 1, comprising the structure: 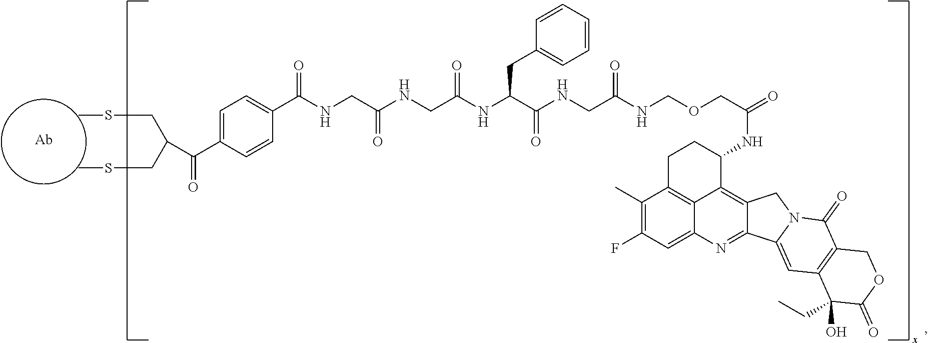
where x is selected from about 1, about 2, about 3, about 4, about 5, about 6, about 7 and about 8.

14. The immunoconjugate of claim 1, comprising the structure: 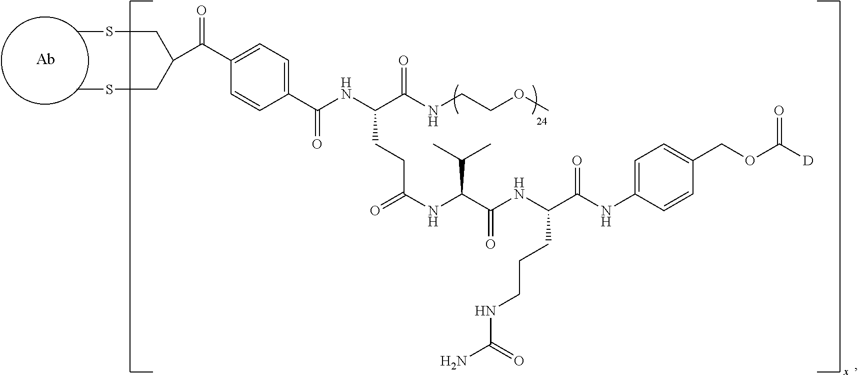
where D is a toxin, and
x is selected from about 1, about 2, about 3, about 4, about 5, about 6, about 7 and about 8.

15. The immunoconjugate of claim 1, comprising the structure: 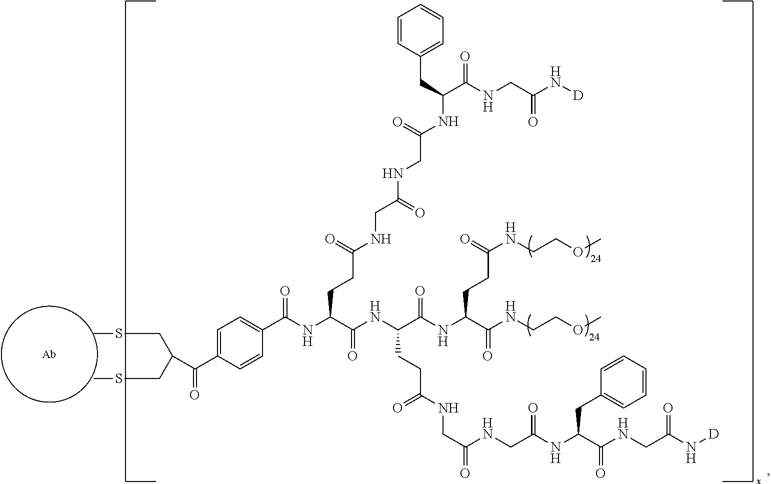
where D is a toxin and
x is selected from about 1, about 2, about 3, about 4, about 5, about 6, about 7 and about 8.

16. The immunoconjugate of claim 1, comprising the structure: 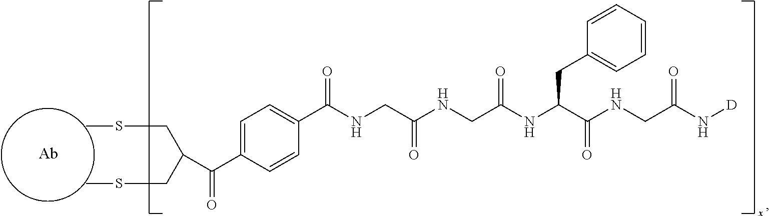
where D is a toxin, and
x is selected from about 1, about 2, about 3, about 4, about 5, about 6, about 7 and about 8.

17. The immunoconjugate of claim 1, wherein D is selected from the group consisting of: maytansinoids, an auristatins selected from MMAE, MMAF and mertansine, taxoids, DNA binders selected from calicheamicins, topoisomerase inhibitors selected from SN-38, Exatecan and DXd, camptothecins, CC1065 analogs, duocarmycins, amatoxins, dexamethone, and budesonide.

18. The immunoconjugate of claim 1, wherein the cytotoxic drug is MMAE, MMAF, a topoisomerase inhibitor, Exatecan, or the 2-hydroxyacetylamide of Exatecan (DXd).

19. The immunoconjugate of claim 1, wherein the cytotoxic drug is DXd.

20. The immunoconjugate of claim 13, wherein x is about 4.

21. A method of treating cancer, comprising administering to a patient in need thereof a therapeutically effective amount of an immunoconjugate of claim 1.

22. The method of claim 21, wherein said cancer is selected from bladder cancer, breast cancer, cervical cancer, colon cancer, colorectal cancer, endometrial cancer, kidney cancer, lung cancer, esophageal cancer, ovarian cancer, prostate cancer, pancreatic cancer, skin cancer, stomach (gastric) cancer, testicular cancer, leukemias and lymphomas.

23. The method of claim 22, wherein the cancer is selected from breast cancer, prostate cancer and colorectal cancer.

24. A method of treating a disease or condition associated with the expression or overexpression of GRP78, said method comprising administering to a patient in need thereof a therapeutically effective amount of an immunoconjugate of claim 1.

25. A nucleic acid that encodes the antibody, or antigen binding fragment thereof, of claim 1, wherein said nucleic acid comprises:
a. SEQ ID NO: 27, SEQ ID NO: 29, or both SEQ ID NO: 27 and SEQ ID NO: 29;
b. SEQ ID NO: 28, SEQ ID NO: 30, or both SEQ ID NO: 28 and SEQ ID NO: 30; or
c. SEQ ID NOS: 26, 31 and 32, SEQ ID NOS: 33, 34 and 35, or all of SEQ ID NOS: 26, 31, 32, 33, 34 and 35;
or an antibody or antigen binding fragment encoded by said nucleic acid.

26. A vector comprising the nucleic acid of claim 27, or a host cell comprising said vector.

27. A process for producing an antibody, comprising cultivating the host cell of claim 30 and recovering the antibody from a culture.

28. A process for producing the immunoconjugate, comprising: (i) linking L to D; (ii) conjugating the L-D to the antibody recovered from the culture of claim 28; and (iii) purifying said immunoconjugate.

29. A process for producing the immunoconjugate of claim 1, comprising conjugating a linker-drug moiety selected from: 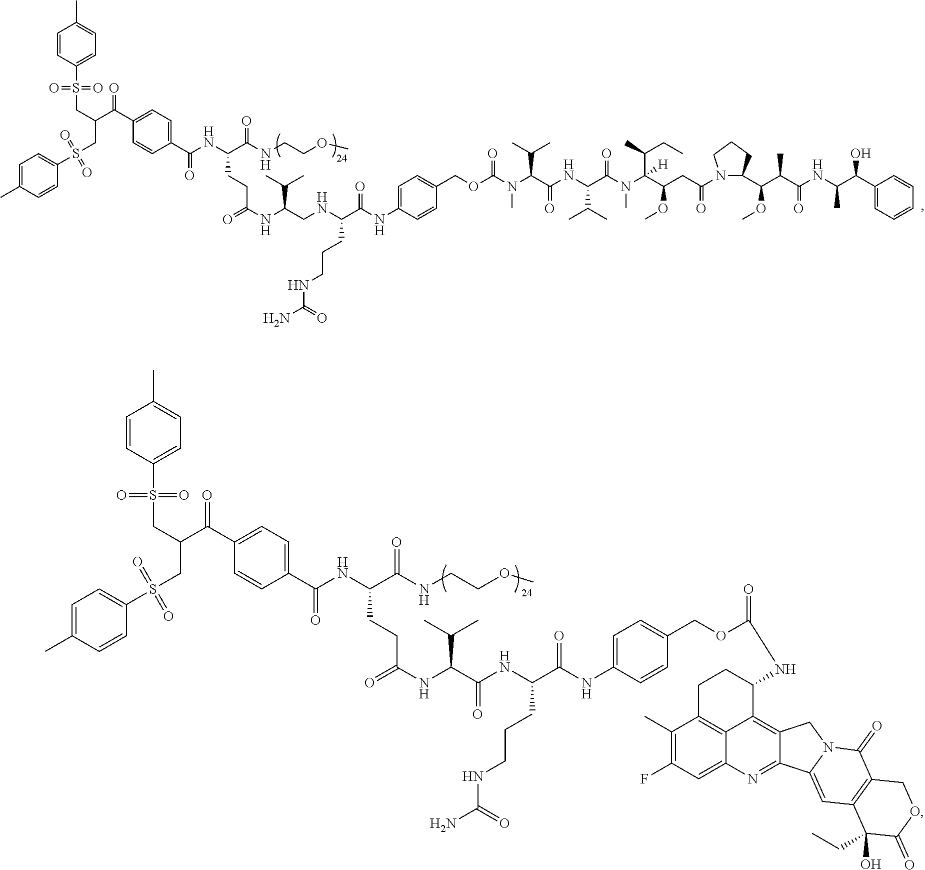 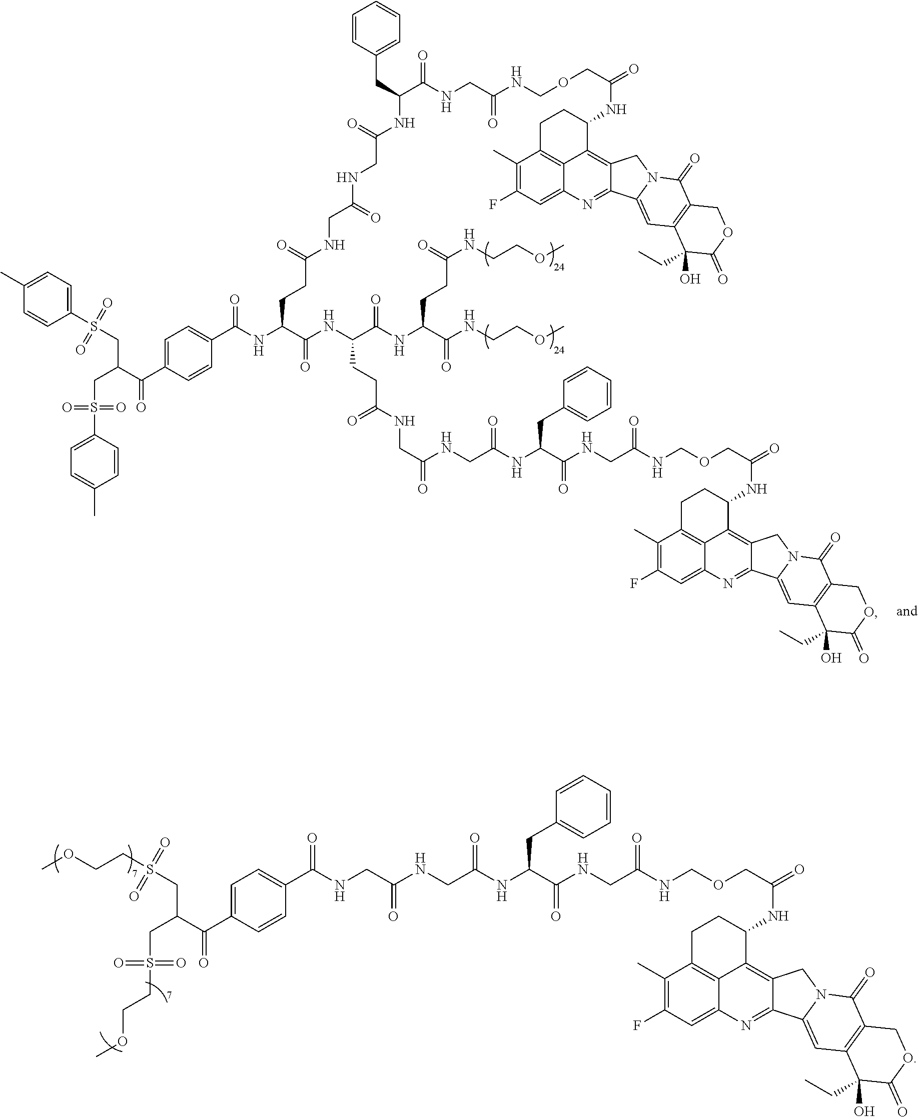
to said antibody.

30. A linker-drug moiety selected from: [chemical structure]
